SEQUENCE LISTING (33 sequences)

SEQ ID NO: 1 - protein, 214 aa, Bos taurus
MKLLILTCLVAVALARPKHPIKHQGLPQEVLNENLLRFFVAPFPEVFGKEKVNELSKDIGSESTEDQAMEDIKQMEAESISSSEEIVPNSVEQKHIQKDDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSGKTTMPLW

SEQ ID NO: 2 - protein, 222 aa, Bos taurus
MKFFIFTCLLAVALAKNTMEHVSSSEESIISQETYKQEKNMAINPSKENLCSTFCKEVVRNANEEEYSIGSSSEESAVVATEEVKITVDDKHYQKALNEINQFYQKFPQYLQYLYQGPIVLNPWDQVKRNAVPITPTLNREQLSTSEENSKKTVDMESTEVFTKKTKLTEEEKNRLNFLKKISQRYQKFALPQYLKTVYQHQKAMEPWIQPKTKVIPYVRYL

SEQ ID NO: 3 - protein, 259 aa, Bos taurus
MPLNTIYKQPQNQIIIHSAPPSLLVLYFGKKELRAMKVLILACLVALALARELEELNVPGEIVESLSSSEESITRINKKIEKFQSEEQQQTEDELQDKIHPFAQTQSLVYPFPGPIHNSLPQNIPPLTQTPVVVPPFLQPEVMGVSKVKEAMAPKHKEMPFPKYPVEPFTERQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 4 - protein, 216 aa, Bos taurus
MKVLILACLVALALARELEELNVPGEIVESLSSSEKIEKFQSEEQQQTEDELQDKIHPFAQTQSLVYPFPGPIHNSLPQNIPPLTQTPVVVPPFLQPEVMGVSKVKEAMAPKHKEMPFPKYPVEPFTERQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 5 - protein, 190 aa, Bos taurus
MMKSFFLVVTILALTLPFLGAQEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTIEAVESTVATLEASPEVIESPPEINTVQVTSTAV

SEQ ID NO: 6 - DNA, 23 bp, synthetic construct
  source 1..23: mol_type = other DNA; note = synthetic oligonucleotide
tccctcaaagcaacaacaacccc

SEQ ID NO: 7 - RNA, 23 nt, Homo sapiens
  source 1..23: mol_type = other RNA
agctggtgttgtgaatcaggccg

SEQ ID NO: 8 - DNA, 21 bp, synthetic construct
  source 1..21: mol_type = other DNA; note = synthetic oligonucleotide
gaggctaagaggcgggaggat

SEQ ID NO: 9 - RNA, 22 nt, Homo sapiens
  source 1..22: mol_type = other RNA
tctcccaacccttgtaccagtg

SEQ ID NO: 10 - RNA, 22 nt, Homo sapiens
  source 1..22: mol_type = other RNA
tgaggtagtaagttgtattgtt

SEQ ID NO: 11 - RNA, 21 nt, synthetic construct
  source 1..21: mol_type = other RNA; note = synthetic oligonucleotide
tccctcaaagcaacaaccccc

SEQ ID NO: 12 - RNA, 21 nt, synthetic construct
  source 1..21: mol_type = other RNA; note = synthetic oligonucleotide
gaggctaagaggcgggaggat

SEQ ID NO: 13 - RNA, 21 nt, synthetic construct
  source 1..21: mol_type = other RNA; note = synthetic oligonucleotide
acttccctcaaagcaacaacc

SEQ ID NO: 14 - RNA, 21 nt, synthetic construct
  source 1..21: mol_type = other RNA; note = synthetic oligonucleotide
tccctcaaagcaacaaccccc

SEQ ID NO: 15 - RNA, 21 nt, synthetic construct
  source 1..21: mol_type = other RNA; note = synthetic oligonucleotide
gaggctaagaggcgggaggat

SEQ ID NO: 16 - RNA, 21 nt, synthetic construct
  source 1..21: mol_type = other RNA; note = synthetic oligonucleotide
acttccctcaaagcaacaacc

SEQ ID NO: 17 - RNA, 200 nt, synthetic construct
  source 1..200: mol_type = other RNA; note = synthetic oligonucleotide
ggccgggcgcggtggctcacgcctgtaatcccagctctcagggaggctaagaggcgggaggatagcttgagcccaggagttcgagacctgcctgggcaatatagcgagaccccgttctccagaaaaaggaaaaaaaaaaacaaaagacaaaaaaaaaataagcgtaacttccctcaaagcaacaaccccccccccccttt

SEQ ID NO: 18-19 - skipped (2 entries)

SEQ ID NO: 20 - DNA, 40 bp, synthetic construct
  source 1..40: mol_type = other DNA; note = sense
aaactagcggccgctagtgaggctaagaggcgggaggatt

SEQ ID NO: 21 - DNA, 44 bp, synthetic construct
  source 1..44: mol_type = other DNA; note = antisense
ctagaatcctcccgcctcttagcctcactagcggccgctagttt

SEQ ID NO: 22 - DNA, 40 bp, synthetic construct
  source 1..40: mol_type = other DNA
aaactagcggccgctagttccctcaaagcaacaaccccct

SEQ ID NO: 23 - DNA, 44 bp, synthetic construct
  source 1..44: mol_type = other DNA; note = antisense
ctagagggggttgttgctttgagggaactagcggccgctagttt

SEQ ID NO: 24 - DNA, 40 bp, synthetic construct
  source 1..40: mol_type = other DNA; note = sense
aaactagcggccgctagtacttccctcaaagcaacaacct

SEQ ID NO: 25 - DNA, 44 bp, synthetic construct
  source 1..44: mol_type = other DNA; note = antisense
ctagaggttgttgctttgagggaagtactagcggccgctagttt

SEQ ID NO: 26 - DNA, 40 bp, synthetic construct
  source 1..40: mol_type = other DNA; note = sense
aaactagcggccgctagttccctcaaagcatgttgcccct

SEQ ID NO: 27 - DNA, 44 bp, synthetic construct
  source 1..44: mol_type = other DNA; note = antisense
ctagaggggcaacatgctttgagggaactagcggccgctagttt

SEQ ID NO: 28 - DNA, 40 bp, synthetic construct
  source 1..40: mol_type = other DNA; note = sense
aaactagcggccgctagtgaggctttcacgcccctcgatt

SEQ ID NO: 29 - DNA, 44 bp, synthetic construct
  source 1..44: mol_type = other DNA; note = antisense
ctagaatcgaggggcgtgaaagcctcactagcggccgctagttt

SEQ ID NO: 30 - DNA, 40 bp, synthetic construct
  source 1..40: mol_type = other DNA; note = sense
aaactagcggccgctagtacttccctcaaagcatgttgct

SEQ ID NO: 31 - DNA, 44 bp, synthetic construct
  source 1..44: mol_type = other DNA; note = antisense
ctagagcaacatgctttgagggaagtactagcggccgctagttt

SEQ ID NO: 32-33 - skipped (2 entries)

(19) United States
(12) Patent Application Publication
(10) Pub. No.: US 2026/0053852 A1
(43) Pub. Date: Feb. 26, 2026

(54) NONCODING RNA AGENTS (BCYRN1 AND DERIVATIVES THEREOF) TO TREAT DISEASES OF IMMUNITY

(71) Applicant: Cedars-Sinai Medical Center, Los Angeles, CA (US)

(72) Inventors: Eduardo Marbán, Santa Monica, CA (US); Ke Liao, Alhambra, CA (US)

(21) Appl. No.: 18/874,061

(22) PCT Filed: Jun. 13, 2023
(86) PCT No.: PCT/US2023/068362

§ 371 Date: Dec. 11, 2024

Related U.S. Application Data
(60) Provisional application No. 63/366,459, filed on Jun. 15, 2022.

(51) Int. Cl. A61K 35/17; A61K 31/712; A61K 40/11; A61K 40/22; A61P 9/10; C12N 5/0783; C12N 15/113

(52) U.S. Cl. CPC A61K 35/17; A61K 31/712; A61K 40/11; A61K 40/22; A61P 9/10; C12N 5/0637; C12N 15/113; C12N 2310/14; C12N 2310/321; C12N 2320/31

(57) ABSTRACT

Provided herein are therapeutic compositions containing a noncoding RNA BCYRN1 or a derivative thereof, such as a nucleic acid containing a BCYRN1-derived binding sequence (BDSS). Also provided are methods of treating an immune-related disorder by administering a therapeutically effective amount of a composition containing at least one nucleic acid having a BDSS to a subject in need thereof.

### CROSS REFERENCE TO RELATED APPLICATIONS

This application claims the benefit of U.S. Provisional Application No. 63/366,459, filed on Jun. 15, 2022, which is hereby incorporated by reference in its entirety.

### STATEMENT REGARDING FEDERALLY SPONSORED R&D

This invention was made with government support under Grant No. R01 HL124074, awarded to Dr. Eduardo Marbin by the National Institutes of Health. The Government has certain rights in the invention.

### REFERENCE TO SEQUENCE LISTING

The present application is being filed along with a Sequence Listing in electronic format. The Sequence Listing is provided as a file entitled CSMC020WO_SEQLIST.xml created on Jun. 13, 2023, which is 32,404 bytes in size. The information in the electronic format of the Sequence Listing is incorporated herein by reference in its entirety.

### BACKGROUND

### Field

The present disclosure relates to therapeutic RNA, derivatives and sequence variants thereof, and treatment of immune-related disorders or inflammatory conditions using same.

Inflammation and tissue injury are main drivers of pathology in certain cardiac injury and other diseases. A growing body of evidence suggests that regulatory T cells (Tregs) favor tissue repair and functional recovery. In particular, the subset of CD4+Foxp3+ Tregs has the potential to mitigate injury via release of inhibitory cytokines (e.g., IL-10), growth factors (e.g. TGFβ), or contact-dependent effects.

### SUMMARY

Provided herein is a therapeutic composition comprising at least one nucleic acid comprising a BDSS (BCYRN1-derived binding sequence), the BDSS comprising a nucleotide sequence as provided by any one of SEQ ID NOs: 11-16, or a sequence at least 90% identical thereto, wherein each of the at least one nucleic acid is no more than 35 nucleotides in length. In several embodiments, the BDSS comprises a nucleotide sequence as provided by any one of SEQ ID NOs: 11-16, or a sequence at least 95% identical thereto. In several embodiments, the BDSS comprises a nucleotide sequence as provided by any one of SEQ ID NOs: 11-16. In several embodiments, the composition comprises at least one of: a first nucleic acid comprising BDSS-138 (rUrCrCrCrUrCrArArArGrCrArArCrArArCrCrCrCrC) (SEQ ID NO: 14), or a sequence at least 90% identical thereto, wherein the first nucleic acid is no more than 35 nucleotides in length; a second nucleic acid comprising BDSS-150 (rGrArGrGrCrUrArArGrArGrGrCrGrGrGrArGrGrArU) (SEQ ID NO: 15), or a sequence at least 90% identical thereto, wherein the second nucleic acid is no more than 35 nucleotides in length; and a third nucleic acid comprising BDSS-98 (rArCrUrUrCrCrCrUrCrArArArGrCrArArCrArArCrC) (SEQ ID NO: 16), or a sequence at least 90% identical thereto, wherein the third nucleic acid is no more than 35 nucleotides in length. In several embodiments, the composition comprises at least two of the first, second and third nucleic acids. the composition comprises the first, second and third nucleic acids. In several embodiments, the first nucleic acid consists of or consists essentially of BDSS-138 (rUrCrCrCrUrCrArArArGrCrArArCrArArCrCrCrCrC) (SEQ ID NO: 14), the second nucleic acid consists of or consists essentially of BDSS-150 (rGrArGrGrCrUrArArGrArGrGrCrGrGrGrArGrGrArU) (SEQ ID NO: 15), and the third nucleic acid consists of or consists essentially of BDSS-98 (rArCrUrUrCrCrCrUrCrArArArGrCrArArCrArArCrC) (SEQ ID NO: 16).

In several embodiments, the composition comprises at least one of: a first nucleic acid comprising a BDSS comprising: UCCCUCAAAGCAACAACCCCC (SEQ ID NO: 11), or a sequence at least 90% identical thereto, wherein the first nucleic acid is no more than 35 nucleotides in length; a second nucleic acid comprising a BDSS comprising: GAGGCUAAGAGGCGGGAGGAU (SEQ ID NO: 12), or a sequence at least 90% identical thereto, wherein the second nucleic acid is no more than 35 nucleotides in length; and a third nucleic acid comprising a BDSS comprising: ACUUCCCUCAAAGCAACAACC (SEQ ID NO: 13), or a sequence at least 90% identical thereto, wherein the third nucleic acid is no more than 35 nucleotides in length. In several embodiments, the composition comprises at least two of the first, second and third nucleic acids. In several embodiments, the composition comprises the first, second and third nucleic acids. In several embodiments, the first nucleic acid comprises a BDSS consisting of or consisting essentially of: UCCCUCAAAGCAACAACCCCC (SEQ ID NO: 11), the second nucleic acid comprises a BDSS consisting of or consisting essentially of: GAGGCUAAGAGGCGGGAGGAU (SEQ ID NO: 12), and the third nucleic acid comprises a BDSS consisting of or consisting essentially of: ACUUCCCUCAAAGCAACAACC (SEQ ID NO: 13).

Also provided is a therapeutic composition comprising at least one BDSS (BCYRN1-derived binding sequence) compound, each comprising a nucleotide sequence as provided by any one of SEQ ID NOs: 11-16, or a derivative thereof having 1, 2, 3, 4, or 5 substitutions thereto, for the treatment of a disease of immunity and/or inflammation. In several embodiments, the at least one BDSS compound each comprises at least one of: BDSS-138 (rUrCrCrCrUrCrArArArGrCrArArCrArArCrCrCrCrC) (SEQ ID NO: 14), BDSS-150 (rGrArGrGrCrUrArArGrArGrGrCrGrGrGrArGrGrArU) (SEQ ID NO: 15) or BDSS-98 (rArCrUrUrCrCrCrUrCrArArArGrCrArArCrArArCrC) (SEQ ID NO: 16), or a derivative thereof having 1, 2, 3, 4, or 5 substitutions thereto. In several embodiments, the at least one BDSS compound each comprises a nucleotide sequence comprising: UCCCUCAAAGCAACAACCCCC (SEQ ID NO: 11), GAGGCUAAGAGGCGGGAGGAU (SEQ ID NO: 12), or ACUUCCCUCAAAGCAACAACC (SEQ ID NO: 13), or a derivative thereof having 1, 2, 3, 4, or 5 substitutions thereto. In several embodiments, the at least one BDSS compound comprises a nucleotide sequence comprising: UCCCUCAAAGCAACAACCCCC (SEQ ID NO: 11), GAGGCUAAGAGGCGGGAGGAU (SEQ ID NO: 12), or ACUUCCCUCAAAGCAACAACC (SEQ ID NO: 13), or a derivative thereof having at least 80% sequence identity thereto.

Also provided is a therapeutic composition comprising at least one inhibitory nucleic acid that each binds to at least one of miR-138, miR-150 and miR-98, wherein the at least one nucleic acid is at most 35 nucleotides long. In several embodiments, the composition comprises at least one of: a first inhibitory nucleic acid that binds to miR-138; a second inhibitory nucleic acid that binds to miR-150; and a third inhibitory nucleic acid that binds to miR-98, wherein each of the first, second and third inhibitory nucleic acids are at most 35 nucleotides long. In several embodiments, the composition comprises at least two of the first, second and third inhibitory nucleic acids. In several embodiments, the composition comprises the first, second and third inhibitory nucleic acids.

In several embodiments, the inhibitory nucleic acid that binds to miR-138 or miR-98 comprises ACAAC (SEQ ID NO: 18). In several embodiments, the inhibitory nucleic acid that binds to miR-150 comprises GGGAG (SEQ ID NO: 19). In several embodiments, the nucleic acid or BDSS compound comprises ACAAC (SEQ ID NO: 18). In several embodiments, the nucleic acid or BDSS compound comprises GGGAG (SEQ ID NO: 19).

In several embodiments, the at least one nucleic acid or BDSS compound comprises RNA. In several embodiments, the at least one nucleic acid or BDSS compound comprises at least one chemically modified nucleotide. In several embodiments, the at least one chemically-modified nucleotide comprises a backbone modification. In several embodiments, the at least one chemically-modified nucleotide comprises a locked nucleic acid (LNA) and/or a methylation.

Provided herein is a therapeutic composition comprising the following long non-coding RNA or one or more synthetic derivatives thereof: GGCCGGGCGCGGUGGCUCACGCCUGUAAUCCCAGCUCUCAGGGAGGCUAAGAG GCGGGAGGAUAGCUUGAGCCCAGGAGUUCGAGACCUGCCUGGGCAAUAUAGC GAGACCCCGUUCUCCAGAAAAAGGAAAAAAAAAAACAAAAGACAAAAAAAAA AUAAGCGUAACUUCCCUCAAAGCAACAACCCCCCCCCCCCUUU (SEQ ID NO: 17), wherein the derivative is no more than 35 nucleotides in length, and wherein the long non-coding RNA or synthetic derivatives thereof enhances Treg functionality.

Also provided is a therapeutic composition comprising: a first BDSS (BCYRN1-derived binding sequence) compound as provided by SEQ ID NO: 11, wherein the first BDSS compound is no more than 35 nucleotides in length; a second BDSS compound as provided by SEQ ID NO:12, wherein the second BDSS compound is no more than 35 nucleotides in length; a third BDSS compound as provided by SEQ ID NO: 13, wherein the third BDSS compound is no more than 35 nucleotides in length; and a pharmaceutically acceptable excipient.

In several embodiments, the composition comprises a transfection reagent. In several embodiments, the transfection reagent comprises one or more of a liposome, lipid nanoparticle (LNP), an extracellular vesicle (EV), and a polyethylene glycol (PEG)-cationic lipid complex (PCLC). In several embodiments, the transfection reagent comprises EV derived from cardiosphere-derived cells (CDC).

Also provided is an isolated nucleic acid comprising a nucleotide sequence of UCCCUCAAAGCAACAACCCCC (SEQ ID NO: 11), wherein the nucleic acid is RNA, wherein the nucleic acid is at most 35 nucleotides long. Further provided is an isolated nucleic acid comprising a nucleotide sequence of GAGGCUAAGAGGCGGGAGGAU (SEQ ID NO: 12), wherein the nucleic acid is RNA, wherein the nucleic acid is at most 35 nucleotides long. Provided herein is an isolated nucleic acid comprising a nucleotide sequence of ACUUCCCUCAAAGCAACAACC (SEQ ID NO: 13), wherein the nucleic acid is RNA, wherein the nucleic acid is at most 35 nucleotides long. Also provided is an isolated nucleic acid comprising a nucleotide sequence at least 95% identical to any one of SEQ ID NOs: 11-13, wherein the nucleic acid is RNA, wherein the nucleic acid is at most 35 nucleotides long.

In several embodiments, the nucleic acid comprises at least one chemically modified nucleotide. In several embodiments, the at least one chemically-modified nucleotide comprises a backbone modification. In several embodiments, the backbone modification comprises a backbone sugar modification. In several embodiments, the at least one chemically-modified nucleotide comprises a locked nucleic acid (LNA) and/or a methylated nucleotide.

Provided herein is a vector comprising a nucleic acid sequence encoding an RNA comprising at least one of the following sequences: UCCCUCAAAGCAACAACCCCC (SEQ ID NO: 11), GAGGCUAAGAGGCGGGAGGAU (SEQ ID NO: 12), and ACUUCCCUCAAAGCAACAACC (SEQ ID NO: 13). Also provided is a collection of vectors comprising: a first vector comprising a nucleic acid sequence encoding a first RNA comprising: UCCCUCAAAGCAACAACCCCC (SEQ ID NO: 11), wherein the first RNA is at most 35 nucleotides long; a second vector comprising a nucleic acid sequence encoding a second RNA comprising: GAGGCUAAGAGGCGGGAGGAU (SEQ ID NO: 12), wherein the second RNA is at most 35 nucleotides long; and a third vector comprising a nucleic acid sequence encoding a second RNA comprising: ACUUCCCUCAAAGCAACAACC (SEQ ID NO: 13), wherein the second RNA is at most 35 nucleotides long. In several embodiments, the vector is a lentiviral vector.

Provided herein is a regulatory T cell comprising any one of the nucleic acid of the present disclosure, wherein an anti-inflammatory activity of the regulatory T cell is increased compared to a regulatory T cell without the nucleic acid. Also provided is a regulatory T cell genetically modified to comprise any one of the vector or the collection of vectors of the present disclosure. In several embodiments, the cell is in a subject. In several embodiments, the cell is in culture.

Also provided is a cardiosphere-derived cell (CDC) genetically modified to comprise any one of the vector or the collection of vectors of the present disclosure.

Also provided is a method of treating an immune-related disorder, comprising administering a therapeutically effective amount of any one of the compositions or any one of the nucleic acids of the present disclosure to a subject in need thereof, to thereby treat an immune-related disorder.

Also provided is a method of treating an immune-related disorder, comprising administering a therapeutically effective amount of a noncoding RNA BCYRN1 or a derivative thereof to a subject in need thereof. In several embodiments, the method comprises administering a therapeutically effective amount of a composition comprising at least one BDSS (BCYRN1-derived binding sequence) compound comprising a nucleic acid sequence selected from SEQ ID NO: 11-13 to the subject in need thereof. In several embodiments, the noncoding RNA BCYRN1 or a derivative thereof comprises one or more nucleic acid sequences, or a sequence having 1, 2, 3, 4, or 5 substitutions thereto, selected from: UCCCUCAAAGCAACAACCCCC (SEQ ID NO: 11), GAGGCUAAGAGGCGGGAGGAU (SEQ ID NO: 12), ACUUCCCUCAAAGCAACAACC (SEQ ID NO: 13). In several embodiments, the noncoding RNA BCYRN1 or a derivative thereof comprises one or more nucleic acid sequences, or a sequence at least 80% identical thereto, selected from: UCCCUCAAAGCAACAACCCCC (SEQ ID NO: 11), GAGGCUAAGAGGCGGGAGGAU (SEQ ID NO: 12), ACUUCCCUCAAAGCAACAACC (SEQ ID NO: 13). In several embodiments, the noncoding RNA BCYRN1 or a derivative thereof has a length of between about 15-50 nt.

In several embodiments, the immune-related disorder comprises an inflammatory disorder. In several embodiments, the immune-related disorder comprises a cardiac immune-related disorder, an autoimmune disease, or transplant rejection. In several embodiments, the cardiac immune-related disorder comprises myocarditis. In several embodiments, the immune-related disorder comprises one or more of myocarditis, myocardial infarction, autoimmune conditions or an inflammatory conditions associated with a viral infection. In several embodiments, the method comprises oral, intravenous, intramuscular, intracardial, airway (aerosol), or pulmonary administration. In several embodiments, the immune-related disorder is secondary to a viral infection.

Also provided is a method of modulating regulatory T-cell (Treg) activity, comprising contacting Tregs with an effective amount of a noncoding RNA BCYRN1 or a derivative thereof, to thereby modulate the activity of the Tregs. In several embodiments, the method includes contacting Tregs with a noncoding RNA BCYRN1 or a derivative thereof to thereby increase proliferation, migration and/or IL-10 production by the Tregs. In several embodiments, the contacting comprises administering the effective amount of a noncoding RNA BCYRN1 or a derivative thereof to a subject, thereby modulating the activity of the Tregs in the subject.

Also provided is a use of the composition of any one of the compositions or any one of the nucleic acids of the present disclosure for treatment of an immune-related disorder in a subject in need thereof.

Also provided is a use of the composition of any one of the compositions or any one of the nucleic acids of the present disclosure for preparation of a medicament for treating an immune-related disorder in a subject in need thereof.

Also provided is a use of a nucleic acid for treating an immune-related disorder, the nucleic acid comprising: a long non-coding RNA comprising the following nucleotide sequence, or a synthetic derivative thereof: GGCCGGGCGCGGUGGCUCACGCCUGUAAUCCCAGCUCUCAGGGAGGCUAAGAG GCGGGAGGAUAGCUUGAGCCCAGGAGUUCGAGACCUGCCUGGGCAAUAUAGC GAGACCCCGUUCUCCAGAAAAAGGAAAAAAAAAAACAAAAGACAAAAAAAAA AUAAGCGUAACUUCCCUCAAAGCAACAACCCCCCCCCCCCUUU (SEQ ID NO: 17), wherein the derivative is no more than 35 nucleotides in length, and wherein the long non-coding RNA or synthetic derivative thereof enhances Treg functionality.

Also provided is the administration of noncoding RNA BCYRN1 or a derivative thereof as a therapeutic agent for diseases of immunity and inflammation.

Also provided is a method of treating an immune-related disorder comprising administering a therapeutically effective amount of an inhibitor of miR-138, miR-150 and/or miR-98 to a subject in need thereof. In several embodiments, the inhibitor is BCYRN1 or a derivative thereof.

Also provided a kit comprising: the composition of any one of the compositions, any one of the nucleic acids, or any one of the vectors or the collection of vectors of the present disclosure; and a transfection reagent. The kit can include any suitable transfection reagent, as described herein. In several embodiments, the transfection reagent comprises one or more of a lipid, a liposome, a lipid nanoparticle (LNP), PEGylated lipid, and an extracellular vesicle (EV). In several embodiments, the kit includes a pharmaceutically acceptable excipient.

### BRIEF DESCRIPTION OF THE DRAWINGS

FIG. 1 is a series of graphs illustrating an assessment of cardiosphere-derived cell extracellular vesicle (CDC-EV) induction of human iTreg cells proliferation, migration, and induction of IL-10. An experimental outline for in vitro Treg cells global transcriptomic analysis is shown in panel A where total CD4+ T cell population was isolated from FOXP3 reporter mice, followed by differentiating the isolated naïve CD4+ T cells into induced Tregs (iTreg). The iTreg cells were exposed to either vehicle or human CDC-EVs for 5 days, and subsequently assessed for transcriptomic changes, as shown in panels B and C. The function heat maps depict enrichment in cell survival (panel B) and cellular movement (panel C) functional categories in the upregulated transcripts in group of CDC-EVs vs Vehicle based on IPA analysis. As shown in panels D-F, human iTreg cells were exposed to either CDC-EVs or fibroblast EVs (NHDF-EVs) at indicated concentration (0-5000 EVs/cell) for 24 hours (panel D), 72 hours (panel E), and 5 days (panel F). The migration of human iTreg cells following exposure to CDC-EVs or NHDF-EVs, towards recombinant 500 ng/mL of recombinant CCL20 was assessed using a 24-well Transwell plate as shown in panel G. Real-time PCR analysis of IL-10 mRNA expression in human iTreg cells exposed to either CDC-EVs or NHDF-EVs for 72 hours as shown in panel H. Protein levels of IL-10 were assayed by ELISA in supernatants of human iTreg cells exposed to either CDC-EVs or NHDF-EVs for 72 hours, as shown in panel I. One-way ANOVA followed by Bonferroni's post hoc test was used to determine the statistical significance among multiple groups. All data are presented as means±SD or SEM of three or four individual experiments (biological replicates). *, P<0.05, **, P<0.01, ***, P<0.001 versus control group

FIG. 2 is a series of graphs illustrating an assessment of CDC-EVs mediating increased expression of BCYRN1 in Treg cells. RNAseq of CDC-EVs shows LncRNA to be plentiful compared to NHDF-EVs, as shown in panel A. List of top 10 plentiful LncRNAs, and BCYRN1 is the highest enriched in CDC-EVs (CDC-EVs (left bars) and NHDF-EVs (right bars)), as shown in panel B. BCYRN1 is expressed more in CDC-EVs than in NHDF-EVs by qPCR, as shown in panel C. Confocal microscopy shows uptake of PKH26-labeled CDC-EVs by human iTregs, as shown in panel D. Assessment of BCYRN1 expression by q-PCR in human iTregs exposed to CDC-EVs or NHDF-EVs for indicated times, as shown in panel E. One-way ANOVA followed by Bonferroni's post hoc test was used to determine the statistical significance among multiple groups. All data are presented as means±SD or SEM of three or four individual experiments (biological replicates). *, P<0.05, **, P<0.01, ***, P<0.001 versus control group.

FIG. 3 is a series of graphs illustrating an assessment of CDC-EVs mediated Treg proliferation, migration and induction of IL-10 involving EV-BCYRN1. Overexpression (OE) of BCYRN1 in human iTreg cells followed assessments of proliferation (panel A), migration (panel B), and IL-10 production (panel C). Transfected CDCs with siRNA-BCYRN1 results in knocking down of BCYRN1 in both CDCs and CDC-EVs, as shown in panels D and E. Exposure of human iTreg cells to CDC-EVs with BCYRN1 knocking down followed assessments of proliferation (panel F), migration (panel G), and IL-10 production (panel H). One-way ANOVA followed by Bonferroni's post hoc test was used to determine the statistical significance among multiple groups. All data are presented as means±SD or SEM of three or four individual experiments (biological replicates). *, P<0.05, **, P<0.01, ***, P<0.001 versus control group. ##, P<0.01 versus si-Ctrl-CDC-EVs group.

FIG. 4 is a series of graphs depicting an assessment of CDC-EV BCYRN1 induction of autophagy by competitively binding with miR-138 to regulate ATG7 expression. Human iTreg cells were exposed to CDC-EVs or non-treated (ctrl), as shown in panel A. The expression of autophagy markers (LC3b, ATG7 and P62) were assessed by WB. As shown in panel B, Human iTreg cells were exposed to ctrl-CDC-EVs, si-BCYRN1 CDC-EVs (EV with BCYRN1 knock-down) or non-treated (ctrl). The expression of autophagy markers (LC3b, ATG7 and P62) were assessed by WB, as shown in panel C. Human iTreg cells were transfected with Vector or OE-BCYRN1 lenti-vector, followed by assessment of autophagy markers by WB, as shown in panel D. Using biotin-labelled BCYRN1 probe to pull down BCYRN1-binded RNAs, followed by assessment of miR-138, negative control (U6 and GAPDH), and positive control (BCYRN1) expression by qPCR, as shown in panel E. Putative LncRNA BCYRN1 binding sites in miR-13 and human iTreg were co-transfected wild type or mutant luciferase reporters with mimic miR-138 into HEK-293T cells, followed by the assessment of relative luciferase activity, as shown in panel F. Co-transfected miR-138 and BCYRN1 in Tregs followed assessments of ATG7 by WB. One-way ANOVA followed by Bonferroni's post hoc test was used to determine the statistical significance among multiple groups. All data are presented as means±SD or SEM of three or four individual experiments (biological replicates). *, P<0.05, **, P<0.01, ***, P<0.001 versus control group.

FIG. 5 is a series of graphs depicting an assessment of CDC-EV BCYRN1 induction of Treg migration by competitively binding with miR-150 to regulate CCR6 expression. Human iTreg cells were exposed to CDC-EVs or non-treated (ctrl). The expression of CCR6 were assessed by qPCR, as shown in panel A. As shown in panel B, human Treg cells were exposed to ctrl-CDC-EVs, si-BCYRN1 CDC-EVs (EV with BCYRN1 knock-down) or non-treated (ctrl). The expression of CCR6 were assessed by qPCR, as shown in panel C. Human Treg cells were transfected with Vector or OE-BCYRN1 lenti-vector, followed by assessment of CCR6 by qPCR, as shown in panel D. Using biotin-labelled BCYRN1 probe to pull down BCYRN1-binded RNAs, followed by assessment of miR-150, negative control (U6 and GAPDH), and positive control (BCYRN1) expression by qPCR, as shown in panel E. Putative LncRNA BCYRN1 binding sites in miR-150. And human iTreg were co-transfected wild type or mutant luciferase reporters with mimic miR-150 into HEK-293T cells, followed by the assessment of relative luciferase activity, as shown in panel F and panel G. Co-transfected miR-150 and BCYRN1 in Tregs followed by assessments of CCR6 by WB (panel F) and cellular migration by trans-well migration assay (panel G). One-way ANOVA followed by Bonferroni's post hoc test was used to determine the statistical significance among multiple groups. All data are presented as means±SD or SEM of three or four individual experiments (biological replicates). *, P<0.05, **, P<0.01, ***, P<0.001 versus control group. ###, P<0.001 versus miR-150 group.

FIG. 6 is a series of graphs illustrating an assessment of BCYRN1 mediated induction of IL-10 in Treg by competitively binding with miR-98 to regulate IL-10 expression. As shown in panel A, biotin-labelled BCYRN1 probe was used to pull down BCYRN1-binded RNAs, followed by assessment of miR-98, negative control (U6 and GAPDH), and positive control (BCYRN1) expression by qPCR (panel B). Putative LncRNA BCYRN1 binding sites in miR-98. And human iTreg were co-transfected wild type or mutant luciferase reporters with mimic miR-98 into HEK-293T cells, followed by the assessment of relative luciferase activity, as shown in panel C and panel D). Co-transfected miR-98 and BCYRN1 in Tregs was followed by assessments of IL-10 by qPCR (panel C) and ELISA (panel D). One-way ANOVA followed by Bonferroni's post hoc test was used to determine the statistical significance among multiple groups. All data are presented as means±SD or SEM of three or four individual experiments (biological replicates). *, P<0.05, **, P<0.01, ***, P<0.001 versus control group. ##, P<0.01, ###, P<0.001 versus miR-150 group.

FIG. 7 is a series of graphs illustrating an assessment of therapeutic efficacy of CDC-EVs, CDC-EV BCYRN1 in I/R mouse model by inducing Treg proliferation, infiltration, and induction of IL-10 in the heart. Schematic representation of in vivo I/R protocol is shown in panel A. Representative flow cytometry plots and quantitative measurements of the CD4+Foxp3+, CD4+ Foxp3+ IL10+, and CD4+Foxp3+Brdu+ population in the heart from CDC-EVs, CDC-EV-BCYRN1- and IMDM (vehicle)-treated animals (n=5 mice per group) are shown in panel B. Representative TTC-stained hearts and quantitative measurements of infarct mass % (n=5 mice per group) from CDC-EVs, CDC-EV-BCYRN1 and vehicle-injected animals at 72 hours (hours) after IR injury are shown in panel C. Plasma cTnI values from CDC-EVs, CDC-EV-BCYRN1- and vehicle-injected animals (n=5 mice per group) at 24 hours after IR injury are shown in panel D. One-way ANOVA followed by Bonferroni's post hoc test was used to determine the statistical significance among multiple groups. *, P<0.05, **, P<0.01, ***, P<0.001 versus control group. #, P<0.05 versus CDC-EVs group.

FIG. 8 is a schematic diagram demonstrating the mechanism by which CDC-EV BCYRN1 mediated regulation of Treg cell, which, in turn, leads to cardiac protection in myocardial infarction (MI). BCYRN1 serves as miRNA sponge mediates the cardioprotective effects of CDC-EVs via promoting Treg infiltration (miR-150/CCR6 dependent migration), proliferation (miR-138/ATG7 dependent autophagy), and IL-10 production (miR-98/IL-10) in the heart.

FIG. 9 depicts an assessment of proliferation of CD8+T cells after co-culture with DCs and hTreg/hTreg-BCYRN1 at various ratios. n=5-6/group.

FIG. 10 is a series of graphs illustrating an assessment of, as shown in panel A, hTregs exposed to admixture of BDSSs (each at 100 nM) then assessed for proliferation (CCK-8), transwell migration and IL-10 (ELISA). As shown in Panel B and panel C, hTregs were exposed to BDSS-98 (B; 0-100 nM) for 72 hours then assessed for IL-10 production. Panel C shows hTregs after exposure to BDSS-150, BDSS-138, or BDSS-98 (100 nM) for 72 hours followed by assessment of IL-10 production.

FIG. 11 is a series of graphs depicting an assessment where a cocktail of 3 BDSSs was cardioprotective against MI in mice, comparably to autophagy inducer rapamycin. Panel A shows a schematic protocol of the experiment. Panel B shows representative TTC-stained heart sections and pooled data for infarct mass (as % of LV; n=5 per group) from BDSSs- and rapamycin-infused animals 72 hours post-MI. Panel C shows Circulating cTnI values BDSSs- and vehicle injected mice (n=5 per group).

FIG. 12 is a series of graphs illustrating an assessment where Treg depletion was found to block BDSSs activity. Panel A shows a schematic of Treg depletion and in vivo I/R protocol. Mice received anti-CD25-antibody (100 μg/mouse/injection i.p.) twice, or isotype control daily for 2 days before T/R. On day 3, TTC assay was used to assess IS (mean±SEM, n=5/group). Panel B shows representative plots showing percentage of CD25+ FoxP3+ in CD4+ T cells (Q2 quadrant). Panel C shows quantitative measurements of infarct mass % (n=5 mice per group) from BDSSs- and Mut-BDSSs-injected animals (WT/Treg depletion) at 72 hours after IR injury.

FIG. 13 depicts an assessment where transfection of CDCs with BCYRN1 overexpression (OE) vector resulted in upregulation of BCYRN1 in both CDCs and CDC-EVs.

FIG. 14 illustrates a schematic of heart infiltrating Treg analysis by flow cytometry.

FIG. 15 illustrates a schematic of encapsulation of GFP mRNA in CDC-EVs. As shown in panel A, the mixture was incubated, then exosomes were immunoprecipitated with anti-CD9, anti-CD63, and anti-CD81. As shown in panel B, pulldown was characterized for size and GFP expression. Panel C shows that exosomes expressed GFP in neonatal rat cardiomyocytes.

### DETAILED DESCRIPTION

Non-coding RNA (ncRNA) in CDC-EVs are implicated in disease-modifying bioactivity of CDC-EVs. BCYRN1 is a ncRNA found in CDC-EVs. BCYRN1 can enhance regulatory T cell proliferation, migration, and/or activation. The nucleotide sequence of BCYRN1 is provided in SEQ ID NO: 17. The present disclosure provides nucleic acids that are BCYRN1-derived binding sequences (BDSS) such as those provided by SEQ ID NO: 11-16. BCYRN1 and derivatives thereof can have strong disease-modifying bioactivity in a number of diseases associated with inflammation, including cardiac immune-related disorders, such as myocarditis, autoimmune diseases, transplant rejection, and inflammation or the immune-related disorders secondary to a viral infection. Without being bound by theory, BCYRN1 and its derivatives are thought to behave as microRNA (miRNA) “sponges” and inhibit miRNA-138, miR-150 and miR-98.

Provided herein is an isolated nucleic acid, e.g., RNA, that can include a nucleotide sequence of SEQ ID NO: 11-17, sequence variants and/or chemical modifications thereof, and methods of use thereof. As used herein, “chemical modification” refers to a chemical difference in the structure of the nucleotides of the nucleic acid relative to the corresponding basic nucleotides (e.g., adenine, guanine, uracil, thymidine, cytosine). A chemical modification can be a natural modification or an artificial modification of the chemical structure of the basic nucleotides. In some embodiments, the isolated nucleic acid includes at least one non-natural chemical modification. In general, the present nucleic acids are 35 nucleotides (nt) long or shorter (e.g., 15-35 nt long, or 21-35 nt long). In some embodiments, the nucleic acid is BCYRN1 (SEQ ID NO: 17), or a sequence variant thereof, or a derivative(s) thereof (e.g., one or more BDSS (BCYRN1-derived binding sequence) compound, such as BDSS-138, BDSS-150, and/or BDSS-98). In general, a derivative, e.g., a sequence variant or chemical modification, of the nucleic acid includes variants and chemical modifications that are functional. Thus, sequence variations and chemical modifications contemplated are those that substantially preserve the therapeutic potency of the original molecule (e.g., having a nucleotide sequence of SEQ ID NO: 11-16). In some embodiments, an isolated nucleic acid of the present disclosure is provided.

Nucleic acids of the present disclosure, e.g., BCYRN1 and/or variants and/or derivatives thereof, find use in treating conditions where inflammation and/or tissue injury are the main drivers of pathology. In some embodiments, the nucleic acids of the present disclosure, e.g., BCYRN1 and/or variants thereof and/or derivatives thereof, treat diseases and conditions that are characterized by inflammation. In some embodiments, conditions treated by nucleic acids of the present disclosure, e.g., BCYRN1 and/or variants thereof and/or derivatives thereof, include, without limitation, inflammatory disease, muscular dystrophy, or cardiac injury. In some embodiments, the present nucleic acids, e.g., BCYRN1 and/or variants thereof and/or derivatives thereof, have cardioprotective effects when administered to a subject suffering from cardiac injury due to, without limitation, myocardial infarction and/or heart failure. Without being bound by theory, the nucleic acids, such as BCYRN1, can increase an anti-inflammatory activity of Tregs, e.g., by promoting Treg cell proliferation, CCR6-dependent Treg migration, and/or secretion of interleukin 10 (IL-10) from Tregs. In some embodiments, nucleic acids of the present disclosure induce changes in expression of one or more gene products and/or epigenetic changes in Tregs that are exposed to the nucleic acids. In some embodiments, conditions, e.g., inflammatory disease, muscular dystrophy, or cardiac injury, treated by the nucleic acids of the present disclosure, e.g., BCYRN1 and/or variants thereof and/or derivatives thereof, are conditions that are responsive to anti-inflammatory effects of IL-10. Without being bound by theory, the nucleic acids, such as BCYRN1, can suppress hypertrophic and/or pro-fibrotic signaling cascades, e.g., in injured tissue.

Without being bound by theory and as noted above, it is thought that BCYRN1, and/or variants thereof and/or derivatives thereof, exhibits its therapeutic effect by behaving as miRNA “sponge” and inhibiting miRNA-138, miR-150 and miR-98. Without being bound by theory, an miRNA sponge may bind to (or hybridizes to) a target miRNA in a sequence-dependent manner, and prevent the bound miRNA from exerting its physiological effects (e.g., repressing expression from a target mRNA). Thus, wherever BCYRN1, and/or variants thereof and/or derivatives thereof, are discussed in the present disclosure, any agent, e.g., nucleic acid, that specifically acts as a miRNA sponge for miRNA-138, miR-150 and miR-98 is also contemplated. In any of the treatment methods, in some embodiments, the method of treatment contemplates administering nucleic acids with miRNA sponge functionality for miRNA-138, miR-150 and miR-98.

In some embodiments, nucleic acids of the present disclosure are chemically modified to increase stability, e.g., in vivo and/or in vitro stability. In some embodiments, nucleic acids of the present disclosure are chemically modified to reduce immunogenicity. In some embodiments, the chemical modification of the nucleic acid increases the therapeutic activity of the nucleic acid. In some embodiments, nucleic acids of the present disclosure have enhanced therapeutic potency compared to endogenously encoded RNA molecules. Nucleic acids of the present disclosure in some embodiments can be provided in a composition (e.g., pharmaceutical compositions) or a kit.

The therapeutic nucleic acids of the present disclosure can be administered by any suitable route, including, without limitation, intravenously or orally. Provided herein are intravenous or oral formulations for administration of nucleic acids of the present disclosure, e.g., BCYRN1, and/or variants thereof and/or derivatives thereof, and the use of same for treatment of an immune-related disorder, or a condition associated with inflammation.

### Definitions

As used herein the term “nucleic acid” or “oligonucleotide” refers to multiple nucleotides (e.g., molecules comprising a sugar (e.g. ribose or deoxyribose) linked to a phosphate group and to an exchangeable organic base, which is either a substituted pyrimidine (e.g. cytosine (C), thymidine (T) or uracil (U)) or a substituted purine (e.g. adenine (A) or guanine (G)). The term includes polynucleosides (i.e. a polynucleotide minus the phosphate) and any other organic base containing polymer. Purines and pyrimidines include but are not limited to adenine, cytosine, guanine, thymidine, inosine, 5-methylcytosine, 2-aminopurine, 2-amino-6-chloropurine, 2,6-diaminopurine, hypoxanthine, and other naturally and non-naturally occurring nucleobases, substituted and unsubstituted aromatic moieties. A nucleic acid can include any other suitable modifications. Thus, the term nucleic acid also encompasses nucleic acids with substitutions or modifications, such as in the bases and/or sugars.

Polypeptide or nucleic acid molecules of the present disclosure may share a certain degree of sequence similarity or identity with the reference molecules (e.g., reference polypeptides or reference polynucleotides), for example, with art-described molecules (e.g., engineered or designed molecules or wild-type molecules). The term “identity” as known in the art, refers to a relationship between the sequences of two or more polypeptides or polynucleotides, as determined by comparing the sequences. In the art, identity also means the degree of sequence relatedness between them as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). Identity of related peptides can be readily calculated by known methods. “% identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Any suitable methods and computer programs for the alignment can be used. It is understood that identity depends on a calculation of percent identity but may differ in value due to gaps and penalties introduced in the calculation. Generally, variants of a particular polynucleotide or polypeptide have at least 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, et al (1997), “Gapped BLAST and PSI-BLAST: a new generation of protein database search programs”. Nucleic Acids Res. 25:3389-3402). Another popular local alignment technique is based on the Smith-Waterman algorithm (Smith, T. F. & Waterman, M. S. (1981) “Identification of common molecular subsequences.” J. Mol. Biol. 147:195-197.) A general global alignment technique based on dynamic programming is the Needleman-Wunsch algorithm (Needleman, S. B. & Wunsch. C. D. (1970) “A general method applicable to the search for similarities in the amino acid sequences of two proteins.” J. Mol. Biol. 48:443-453.). More recently a Fast Optimal Global Sequence Alignment Algorithm (FOGSAA) has been developed that purportedly produces global alignment of nucleotide and protein sequences faster than other optimal global alignment methods, including the Needleman-Wunsch algorithm. Other tools are described herein, specifically in the definition of “identity” below.

The term “identity” refers to the overall relatedness between polymeric molecules, for example, between polynucleotide molecules (e.g. DNA molecules and/or RNA molecules) and/or between polypeptide molecules. Calculation of the percent identity of two polynucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a suitable mathematical algorithm. For example, the percent identity between two nucleic acid sequences can be determined using methods such as those described in Computational Molecular Biology, Lesk, A. M., ed., Oxford University Press, New York, 1988; Biocomputing: Informatics and Genome Projects. Smith. D. W., ed., Academic Press. New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleic acid sequences can be determined using the algorithm of Meyers and Miller (CABIOS, 1989, 4:11-17), which has been incorporated into the ALIGN program (version 2.0) using a PAM 120 weight residue table, a gap length penalty of 12 and a gap penalty of 4. The percent identity between two nucleic acid sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

The term “Watson-Crick base-pairing”, or “base-pairing” refers to the formation of hydrogen bonds between specific pairs of nucleotide bases (“complementary base pairs”). For example, two hydrogen bonds form between adenine (A) and uracil (U), and three hydrogen bonds form between guanine (G) and cytosine (C). One method of assessing the strength of bonding between two polynucleotides is by quantifying the percentage of bonds formed between the guanine and cytosine bases of the two polynucleotides (“GC content”). In some embodiments, the GC content of bonding between two nucleic acids of a multimeric molecule (e.g., a multimeric mRNA molecule) is at least 10%, at least 20%, at least 30%, at least 40%, or at least 50%. In some embodiments, the GC content of bonding between two nucleic acids of a multimeric molecule (e.g., a multimeric mRNA molecule) is between 10% and 70%, about 20% to about 60%, or about 30% to about 60%. The formation of a nucleic acid duplex via bonding of complementary base pairs can also be referred to as “hybridization”. Generally, two nucleic acids sharing a region of complementarity are capable, under suitable conditions, of hybridizing (e.g., via nucleic acid base pairing) to form a duplex structure. A region of complementarity can vary in size. In some embodiments, a region of complementarity ranges in length from about 2 base pairs to about 100 base pairs. In some embodiments, a region of complementarity ranges in length from about 5 base pairs to about 75 base pairs. In some embodiments, a region of complementarity ranges in length from about 10 base pairs to about 50 base pairs. In some embodiments, a region of complementarity ranges in length from about 20 base pairs to about 30 base pairs.

“Isolated” as used herein with reference to an isolated biomolecule, e.g., a nucleic acid, has the ordinary and customary meaning to one of ordinary skill in the art in view of the present disclosure. An isolated biomolecule, e.g., an isolated nucleic acid, is generally in a non-natural environment, or in an environment that the biomolecule would otherwise not have been without human intervention of the biomolecule or its environment. In some embodiments, an isolated biomolecule is not inside a cell or an organism.

“Extracellular vesicle” or “EV” as used herein have their ordinary and customary meaning as understood by one of ordinary skill in the art, in view of the present disclosure. EVs include lipid bilayer structures generated by cells, and include exosomes, microvesicles, epididimosomes, argosomes, exosome-like vesicles, microparticles, promininosomes, prostasomes, dexosomes, texosomes, dex, tex, archeosomes and oncosomes.

“Regulatory T cells” or “Treg cells” are used interchangeably herein and have their customary and ordinary meaning as understood by one of ordinary skill in the art, in view of the present disclosure. T_{reg} cells are anti-inflammatory T cells, and can be characterized by expression of cell surface markers that distinguish T_{reg} cells from other immune cells (e.g., effector T cells, B cells, macrophages, NK cells, etc.). In some embodiments, T_{reg} cells are CD4⁺ and at least one of CD25⁺ and FOXP3⁺. In some embodiments T_{reg} cells are induced T_{reg} cells (iT_{reg} cells). In some embodiments, T_{reg} cells, e.g., iT_{reg} cells, are differentiated in vitro from naïve CD4+ T cells. In some embodiments, T_{reg} cells are T_{reg} cells in vivo.

“Subject,” as used herein refers to any vertebrate animal, including mammals and non-mammals. A subject can include primates, including humans, and non-primate mammals, such as rodents, domestic animals or game animals. Non-primate mammals can include mouse, rat, hamster, rabbit, dog, fox, wolf, cat, horse, cow, pig, sheep, goat, camel, deer, buffalo, bison, etc. Non-mammals can include bird (e.g., chicken, ostrich, emu, pigeon), reptile (e.g., snake, lizard, turtle), amphibian (e.g., frog, salamander), fish (e.g., salmon, cod, pufferfish, tuna), etc. The terms, “individual,” “patient,” and “subject” are used interchangeably herein.

“Administering” as used herein can include any suitable routes of administering a therapeutic agent or composition as disclosed herein. Suitable routes of administration include, without limitation, oral, parenteral, intravenous, intramuscular, subcutaneous, transdermal, airway (aerosol), pulmonary, cutaneous, injection or topical administration. Administration can be local or systemic.

As used herein, “treat” and “treatment” includes curing, improving, ameliorating, reducing the severity of, preventing, slowing the progression of, and/or delaying the appearance of a disease, condition and/or symptoms thereof.

A treatment can be considered “effective,” or “therapeutically effective” as used herein, if one or more of the signs or symptoms of a condition described herein are altered in a beneficial manner, other clinically accepted symptoms are improved, or even ameliorated, or a desired response is induced e.g., by at least 2%, 3%, 4%, 5%, 10%, or more, following treatment according to the methods described herein. Efficacy can be assessed, for example, by measuring a marker, indicator, symptom, and/or the incidence of a condition treated according to the methods described herein or any other measurable parameter appropriate, e.g. exercise endurance. Efficacy can also be measured by a failure of an individual to worsen as assessed by hospitalization, or need for medical interventions (e.g., progression of the disease is halted). Treatment includes any treatment of a disease or condition in an individual or an animal (some non-limiting examples include a human or an animal) and includes: (1) inhibiting the disease or condition, e.g., preventing a worsening of symptoms (e.g. pain or inflammation); or (2) relieving the severity of the disease or condition, e.g., causing regression of symptoms. An effective amount for the treatment of a disease or condition means that amount which, when administered to a subject in need thereof, is sufficient to result in effective treatment as that term is defined herein, for that disease or condition. Efficacy of an agent can be determined by assessing physical indicators of a condition or desired response, (e.g. muscle function, mass or volume). One skilled in the art can monitor efficacy of administration and/or treatment by measuring any one of such parameters, or any combination of parameters.

The term “effective amount” or “therapeutically effective amount” as used herein refers to the amount of a composition or an agent needed to alleviate at least one or more symptom of the disease or condition, and relates to a sufficient amount of therapeutic composition to provide the desired effect. The term “effective amount” or “therapeutically effective amount” can refer to an amount of a composition or therapeutic agent that is sufficient to provide a particular anti-inflammatory and/or cardioprotective effect when administered to a typical subject. An effective amount as used herein, in various contexts, can include an amount sufficient to delay the development of a symptom of the disease or condition, alter the course of a symptom disease or condition (for example but not limited to, slowing the progression of a symptom of the disease or condition), or reverse a symptom of the disease or condition. In some embodiments, the therapeutically effective amount is administered in one or more doses of the therapeutic agent. In some embodiments, the therapeutically effective amount is administered in a single administration, or over a period of time in a plurality of doses.

As used herein, the phrase “physiologically compatible” and “pharmaceutically acceptable” are employed interchangeably herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. As used herein “therapeutic” or “pharmaceutically acceptable” as used herein denote that the item (e.g., composition) is suitable for administration to a subject, e.g., a patient.

Definitions of common terms in cell biology and molecular biology can be found in “The Merck Manual of Diagnosis and Therapy”, 19^{th} Edition, published by Merck Research Laboratories, 2006 (ISBN 0-91 1910-19-0); Robert S. Porter et al. (eds.), The Encyclopedia of Molecular Biology, published by Blackwell Science Ltd., 1994 (ISBN 0-632-02182-9); Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

The singular terms “a,” “an,” and “the” include plural referents unless context clearly indicates otherwise. Similarly, the word “or” is intended to include “and” unless the context clearly indicates otherwise. The abbreviation, “e.g.” is used herein to indicate a non-limiting example. Thus, the abbreviation “e.g.” is synonymous with the term “for example.” The term “about” as used herein to, for example, define the values and ranges of molecular weights means that the indicated values and/or range limits can vary within ±20%, e.g., within ±10%, including within ±5%. The use of “about” before a number includes the number itself. For example, “about 5” provides express support for “5.” Numbers provided in ranges include overlapping ranges and integers in between; for example a range of 1-4 and 5-7 includes for example, 1-7, 1-6, 1-5, 2-5, 2-7, 4-7, 1, 2, 3, 4, 5, 6 and 7.

### Nucleic Acids and BCYRN1-Derived Binding Sequences (BDSS)

Provided herein is an isolated nucleic acid that includes a nucleotide sequence of:

(SEQ ID NO: 17)
GGCCGGGCGCGGUGGCUCACGCCUGUAAUCCCAGCUCUCAGGGAGGCUA
AGAGGCGGGAGGAUAGCUUGAGCCCAGGAGUUCGAGACCUGCCUGGGCA
AUAUAGCGAGACCCCGUUCUCCAGAAAAAGGAAAAAAAAAAACAAAAGA
CAAAAAAAAAAUAAGCGUAACUUCCCUCAAAGCAACAACCCCCCCCCCC
CUUU,

or a variant or derivative thereof. In some embodiments, the nucleic acid is or comprises RNA. In some embodiments, the nucleic acid includes a nucleotide sequence at least 80%, 85%, 90%, 92%, 93%, 94%, 95%, 96%, 98%, 99% identical to SEQ ID NO: 17. In some embodiments, the nucleic acid includes one or more binding sites for (e.g., a sequence that hybridizes to) miR-138, miR-150, or miR-98. In some embodiments, the nucleic acid includes at least ACAAC (SEQ ID NO: 18) and/or GGGAG (SEQ ID NO: 19). In some embodiments, the nucleotide sequence of the nucleic acid is, or comprises, SEQ ID NO: 17, or a sequence variant thereof. In some embodiments, the nucleotide sequence of the nucleic acid is SEQ ID NO: 17, or a derivative thereof having 1, 2, 3, 4, or 5 substitutions thereto. Also provided are nucleic acids that include subsequences of SEQ ID NO: 17. The subsequence of SEQ ID NO: 17 can be any suitable length. In some embodiments, the subsequence of SEQ ID NO: 17 is, is about, or is at most 35 nucleotides (nt) long. In some embodiments, the subsequence of SEQ ID NO: 17 is, or is about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 nt long, or longer. In some embodiments, the nucleic acid is at most 35 nt long. In some embodiments, the nucleic acid is 15-35 nt long, or 21-35 nt long.

Also provided are BCYRN1-derived binding sequences (BDSS) or BDSS compounds that include subsequences of SEQ ID NO: 17. In some embodiments, a BDSS or BDSS compound includes a contiguous subsequence of SEQ ID NO: 17 that is, is about, or is at least 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 nt long. In some embodiments, a BDSS or BDSS compound (e.g., BDSS-138) includes a subsequence of residues 170-190 of SEQ ID NO: 17. In some embodiments, a BDSS or BDSS compound (e.g., BDSS-150) includes a subsequence of residues 43-63 of SEQ ID NO: 17. In some embodiments, a BDSS or BDSS compound (e.g., BDSS-98) includes a subsequence of residues 167-187 of SEQ ID NO: 17.

In some embodiments, BDSS-138 is or includes rUrCrCrCrUrCrArArArGrCrArArCrArArCrCrCrCrC (SEQ ID NO: 14). As used herein, a representation of a nucleic acid sequence that includes “r” adjacent to one of the bases (A, U, G, or C) denotes that the sugar backbone is ribose (e.g., the nucleic acid containing the sequence is RNA). Without being bound by theory, BDSS-138 can act as a sponge for miR-138, which in turn can suppress expression of ATG-7, which mediates autophagy involved in survival and proliferation of Treg cells. In some embodiments, BDSS-150 is or includes rGrArGrGrCrUrArArGrArGrGrCrGrGrGrArGrGrArU (SEQ ID NO: 15). Without being bound by theory, BDSS-150 can act as a sponge for miR-150, which in turn can suppress expression of CCR6, which mediates migration in Treg cells. In some embodiments, BDSS-98 is or includes rArCrUrUrCrCrCrUrCrArArArGrCrArArCrArArCrC (SEQ ID NO: 16). Without being bound by theory, BDSS-98 can act as a sponge for miR-98, which in turn can suppress expression of IL-10, an anti-inflammatory cytokine produced by Treg cell.

Provided herein is an isolated nucleic acid or a BDSS (e.g., BDSS-138) that includes a nucleotide sequence of UCCCUCAAAGCAACAACCCCC (SEQ ID NO: 11), or a derivative (e.g., sequence variant) thereof. In some embodiments, the nucleic acid is or comprises RNA. In some embodiments, the BDSS is or comprises RNA. In some embodiments, the nucleic acid or the BDSS includes a nucleotide sequence at least 80%, 85%, 90%, 92%, 93%, 94%, 95%, 96%, 98%, 99% identical to UCCCUCAAAGCAACAACCCCC (SEQ ID NO: 11). In some embodiments, the nucleic acid or BDSS includes at least ACAAC (SEQ ID NO: 18). In some embodiments, the nucleotide sequence of the nucleic acid or the BDSS is UCCCUCAAAGCAACAACCCCC (SEQ ID NO: 11), or a sequence variant thereof. In some embodiments, the nucleotide sequence of the nucleic acid or the BDSS is UCCCUCAAAGCAACAACCCCC (SEQ ID NO: 11), or a derivative thereof having 1, 2, 3, 4, or 5 substitutions thereto. The nucleic acid or the BDSS can be any suitable length. In some embodiments, the nucleic acid or the BDSS is, is about, or is at most 35 nucleotides (nt) long. In some embodiments, the nucleic acid or the BDSS is, is about, or is at most 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 nt long. In some embodiments, the nucleic acid or the BDSS compound is about 15-35 nt long, or about 21-35 nt long. In some embodiments, a BDSS (e.g., BDSS-138) includes a nucleotide sequence of UCCCUCAAAGCAACAACCCCC (SEQ ID NO: 11), or a sequence at least 90% identical thereto, wherein the BDSS is no more than 35 nucleotides in length.

Provided herein is an isolated nucleic acid or a BDSS (e.g., BDSS-150) that includes a nucleotide sequence of GAGGCUAAGAGGCGGGAGGAU (SEQ ID NO: 12), or a derivative (e.g., sequence variant) thereof. In some embodiments, the nucleic acid is or comprises RNA. In some embodiments, the BDSS is or comprises RNA. In some embodiments, the nucleic acid or the BDSS includes a nucleotide sequence at least 80%, 85%, 90%, 92%, 93%, 94%, 95%, 96%, 98%, 99% identical to GAGGCUAAGAGGCGGGAGGAU (SEQ ID NO: 12). In some embodiments, the nucleic acid or BDSS includes at least GGGAG (SEQ ID NO: 19). In some embodiments, the nucleotide sequence of the nucleic acid is GAGGCUAAGAGGCGGGAGGAU (SEQ ID NO: 12), or a sequence variant thereof. In some embodiments, the nucleotide sequence of the nucleic acid is GAGGCUAAGAGGCGGGAGGAU (SEQ ID NO: 12), or a derivative thereof having 1, 2, 3, 4, or 5 substitutions thereto. The nucleic acid can be any suitable length. In some embodiments, the nucleic acid is, is about, or is at most 35 nucleotides (nt) long. In some embodiments, the nucleic acid or the BDSS is, is about, or is at most 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 nt long. In some embodiments, the nucleic acid or the BDSS is about 15-35 nt long, or about 21-35 nt long. In some embodiments, a BDSS (e.g., BDSS-150) includes a nucleotide sequence of GAGGCUAAGAGGCGGGAGGAU (SEQ ID NO: 12), or a sequence at least 90% identical thereto, wherein the BDSS is no more than 35 nucleotides in length.

Provided herein is an isolated nucleic acid or a BDSS (e.g., BDSS-98) that includes a nucleotide sequence of ACUUCCCUCAAAGCAACAACC (SEQ ID NO: 13), or a derivative (e.g., sequence variant) thereof. In some embodiments, the nucleic acid is or comprises RNA. In some embodiments, the BDSS is or comprises RNA. In some embodiments, the nucleic acid or the BDSS includes a nucleotide sequence at least 80%, 85%, 90%, 92%, 93%, 94%, 95%, 96%, 98%, 99% identical to ACUUCCCUCAAAGCAACAACC (SEQ ID NO: 13). In some embodiments, the nucleic acid or BDSS includes at least ACAAC (SEQ ID NO: 18). In some embodiments, the nucleotide sequence of the nucleic acid is ACUUCCCUCAAAGCAACAACC (SEQ ID NO: 13), or a sequence variant thereof. In some embodiments, the nucleotide sequence of the nucleic acid is ACUUCCCUCAAAGCAACAACC (SEQ ID NO: 13), or a derivative thereof having 1, 2, 3, 4, or 5 substitutions thereto. The nucleic acid can be any suitable length. In some embodiments, the nucleic acid or the BDSS is, is about, or is at most 35 nucleotides (nt) long. In some embodiments, the nucleic acid or the BDSS compound is, is about, or is at most 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 nt long. In some embodiments, the nucleic acid or the BDSS is about 21-50 nt long, or about 21-35 nt long. In some embodiments, a BDSS (e.g., BDSS-98) includes a nucleotide sequence of ACUUCCCUCAAAGCAACAACC (SEQ ID NO: 13), or a sequence at least 90% identical thereto, wherein the BDSS is no more than 35 nucleotides in length.

Provided herein is a BDSS compound comprising BDSS-138 (rUrCrCrCrUrCrArArArGrCrArArCrArArCrCrCrCrC) (SEQ ID NO: 14), or a derivative (e.g., sequence variant) thereof. In some embodiments, the BDSS compound includes a nucleotide sequence at least 80%, 85%, 90%, 92%, 93%, 94%, 95%, 96%, 98%, 99% identical to BDSS-138 (rUrCrCrCrUrCrArArArGrCrArArCrArArCrCrCrCrC) (SEQ ID NO: 14). In some embodiments, the BDSS compound includes at least the sequence ACAAC (SEQ ID NO: 18). In some embodiments, the BDSS compound is BDSS-138 (rUrCrCrCrUrCrArArArGrCrArArCrArArCrCrCrCrC) (SEQ ID NO: 14), or a sequence variant thereof. In some embodiments, the BDSS compound is BDSS-138 (rUrCrCrCrUrCrArArArGrCrArArCrArArCrCrCrCrC) (SEQ ID NO: 30), or a derivative thereof having 1, 2, 3, 4, or 5 substitutions thereto. The BDSS compound can include a nucleotide sequence of any suitable length. In some embodiments, the BDSS compound includes a nucleotide sequence that is, is about, or is at most 35 nucleotides (nt) long. In some embodiments, the BDSS compound includes a nucleotide sequence that is, is about, or is at most 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 nt long. In some embodiments, the BDSS compound includes a nucleotide sequence that is about 15-35 nt long, or about 21-35 nt long.

Provided herein is a BDSS compound comprising BDSS-150 (rGrArGrGrCrUrArArGrArGrGrCrGrGrGrArGrGrArU) (SEQ ID NO: 15), or a derivative (e.g., sequence variant) thereof. In some embodiments, the BDSS compound includes a nucleotide sequence at least 80%, 85%, 90%, 92%, 93%, 94%, 95%, 96%, 98%, 99% identical to BDSS-150 (rGrArGrGrCrUrArArGrArGrGrCrGrGrGrArGrGrArU) (SEQ ID NO: 15). In some embodiments, the BDSS compound includes the sequence GGGAG (SEQ ID NO: 19). In some embodiments, the BDSS compound is BDSS-150 (rGrArGrGrCrUrArArGrArGrGrCrGrGrGrArGrGrArU) (SEQ ID NO: 15), or a sequence variant thereof. In some embodiments, the BDSS compound is BDSS-150 (rGrArGrGrCrUrArArGrArGrGrCrGrGrGrArGrGrArU) (SEQ ID NO: 15), or a derivative thereof having 1, 2, 3, 4, or 5 substitutions thereto. The BDSS compound can include a nucleotide sequence of any suitable length. In some embodiments, the BDSS compound includes a nucleotide sequence that is, is about, or is at most 35 nucleotides (nt) long. In some embodiments, the BDSS compound includes a nucleotide sequence that is, is about, or is at most 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 nt long. In some embodiments, the BDSS compound includes a nucleotide sequence that is about 15-35 nt long, or about 21-35 nt long.

Provided herein is a BDSS compound comprising BDSS-98 (rArCrUrUrCrCrCrUrCrArArArGrCrArArCrArArCrC) (SEQ ID NO: 16), or a derivative (e.g., sequence variant) thereof. In some embodiments, the BDSS compound includes a nucleotide sequence at least 80%, 85%, 90%, 92%, 93%, 94%, 95%, 96%, 98%, 99% identical to BDSS-98 (rArCrUrUrCrCrCrUrCrArArArGrCrArArCrArArCrC) (SEQ ID NO: 16). In some embodiments, the BDSS compound includes the sequence ACAAC (SEQ ID NO: 18). In some embodiments, the BDSS compound is BDSS-98 (rArCrUrUrCrCrCrUrCrArArArGrCrArArCrArArCrC) (SEQ ID NO: 16), or a sequence variant thereof. In some embodiments, the BDSS compound is BDSS-98 (rArCrUrUrCrCrCrUrCrArArArGrCrArArCrArArCrC) (SEQ ID NO: 32), or a derivative thereof having 1, 2, 3, 4, or 5 substitutions thereto. The BDSS compound can include a nucleotide sequence of any suitable length. In some embodiments, the BDSS compound includes a nucleotide sequence that is, is about, or is at most 35 nucleotides (nt) long. In some embodiments, the BDSS compound includes a nucleotide sequence that is, is about, or is at most 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 nt long. In some embodiments, the BDSS compound includes a nucleotide sequence that is about 15-35 nt long, or about 21-35 nt long.

Also provided is a inhibitory nucleic acid (e.g., a miRNA sponge) that binds to miR-138, miR-150, and/or miR-98. In some embodiments, the inhibitory nucleic acid binds to miR-138, miR-150, and/or miR-98 and inhibits their function (e.g., prevents repression or promotes expression of one or more genes targeted by the respective miRNA). In some embodiments, the inhibitory nucleic acid includes a nucleotide sequence complementary to at least a portion of one or more of miR-138, miR-150, and/or miR-98, such that the inhibitory nucleic acid binds to (or hybridizes to) the respective miRNA. In some embodiments, the inhibitory nucleic acid includes a nucleotide sequence complementary to a portion of the target miRNA that is, or is at least 5, 6, 7, 8, 9, 10, 11, 12, 13, 14, 15, 16, 17, 18, 19, 20, 21, 22, or 23 nucleotides long. In some embodiments, the inhibitory nucleic acid includes a nucleotide sequence complementary to a contiguous portion of the target miRNA that is, or is at least 5, 6, 7, 8, 9, 10, 11, 12, 13, 14, 15, 16, 17, 18, 19, 20, 21, 22, or 23 nucleotides long. In some embodiments, the inhibitory nucleic acid includes a nucleotide sequence having two or more (e.g., 2, 3, 4, or more) regions of complementarity (e.g., where each region is separated by at least one non-complementary nucleotide) to the target miRNA, where the total length of complementarity is, or is at least 5, 6, 7, 8, 9, 10, 11, 12, 13, 14, 15, 16, 17, 18, 19, 20, or 21 nucleotides long. In some embodiments, the inhibitory nucleic acid includes a nucleotide sequence having two or more (e.g., 2, 3, 4, or more) regions of complementarity (e.g., where each region is separated by at least one non-complementary nucleotide) to corresponding co-linear portions of the target miRNA, where the total length of complementarity is, or is at least 5, 6, 7, 8, 9, 10, 11, 12, 13, 14, 15, 16, 17, 18, 19, 20, or 21 nucleotides long. In some embodiments, the inhibitory nucleic acid includes a nucleotide sequence complementary to a portion of the target miRNA that is about 5-20 nucleotides, about 5-15 nucleotides, or about 5-10 nucleotides long. In some embodiments, the inhibitory nucleic acid includes one or more binding sites for (e.g., a sequence that hybridizes to) miR-138, miR-150, or miR-98. In some embodiments, the inhibitory nucleic acid that binds to miR-138 or miR-98 includes ACAAC (SEQ ID NO: 18). In some embodiments, the inhibitory nucleic acid that binds to miR-150 includes GGGAG (SEQ ID NO: 19).

In some embodiments, the inhibitory nucleic acid includes a nucleotide sequence complementary to at least a portion of miR-138. In some embodiments, the inhibitory nucleic acid binds that binds to miR-138 inhibits suppression of ATG-7 expression by miR-138 in a cell (e.g., a Treg). In some embodiments, miR-138 is human miR-138. In some embodiments, the nucleic acid binds to hsa-miR-138-5p, having a nucleotide sequence of 5′-agcugguguugugaaucaggccg-3′ (SEQ ID NO:7). In some embodiments, the inhibitory nucleic acid includes a nucleotide sequence complementary to a portion of miR-138 (e.g., hsa-miR-138-5p) that is, or is at least 5, 6, 7, 8, 9, 10, 11, 12, 13, 14, 15, 16, 17, 18, 19, 20, 21, 22, or 23 nucleotides long. In some embodiments, the inhibitory nucleic acid includes a nucleotide sequence having two or more (e.g., 2, 3, 4, or more) regions of complementarity (e.g., where each region is separated by at least one non-complementary nucleotide) to miR-138 (e.g., hsa-miR-138-5p), where the total length of complementarity is, or is at least 5, 6, 7, 8, 9, 10, 11, 12, 13, 14, 15, 16, 17, 18, 19, 20, or 21 nucleotides long. In some embodiments, the inhibitory nucleic acid includes a nucleotide sequence having two or more (e.g., 2, 3, 4, or more) regions of complementarity (e.g., where each region is separated by at least one non-complementary nucleotide) to corresponding co-linear portions of miR-138 (e.g., hsa-miR-138-5p), where the total length of complementarity is, or is at least 5, 6, 7, 8, 9, 10, 11, 12, 13, 14, 15, 16, 17, 18, 19, 20, or 21 nucleotides long. In some embodiments, the inhibitory nucleic acid includes a nucleotide sequence complementary to a portion of miR-138 (e.g., hsa-miR-138-5p) that is about 5-20 nucleotides, about 5-15 nucleotides, or about 5-10 nucleotides long. In some embodiments, the nucleic acid includes one or more miR-138 binding sites. In some embodiments, the nucleic acid includes ACAAC (SEQ ID NO: 18).

In some embodiments, the inhibitory nucleic acid includes a nucleotide sequence complementary to at least a portion of miR-150. In some embodiments, the inhibitory nucleic acid binds that binds to miR-150 inhibits suppression of CCR6 expression by miR-150 in a cell (e.g., a Treg). In some embodiments, miR-150 is human miR-150. In some embodiments, the nucleic acid binds to hsa-miR-138-5p, having a nucleotide sequence of 5′-ucucccaacccuuguaccagug-3′ (SEQ ID NO:9). In some embodiments, the inhibitory nucleic acid includes a nucleotide sequence complementary to a portion of miR-150 (e.g., hsa-miR-150-5p) that is, or is at least 5, 6, 7, 8, 9, 10, 11, 12, 13, 14, 15, 16, 17, 18, 19, 20, 21, 22, or 23 nucleotides long. In some embodiments, the inhibitory nucleic acid includes a nucleotide sequence complementary to a contiguous portion of miR-150 (e.g., hsa-miR-150-5p) that is, or is at least 5, 6, 7, 8, 9, 10, 11, 12, 13, 14, 15, 16, 17, 18, 19, 20, 21, 22, or 23 nucleotides long. In some embodiments, the inhibitory nucleic acid includes a nucleotide sequence having two or more (e.g., 2, 3, 4, or more) regions of complementarity (e.g., where each region is separated by at least one non-complementary nucleotide) to miR-150 (e.g., hsa-miR-150-5p), where the total length of complementarity is, or is at least 5, 6, 7, 8, 9, 10, 11, 12, 13, 14, 15, 16, 17, 18, 19, 20, or 21 nucleotides long. In some embodiments, the inhibitory nucleic acid includes a nucleotide sequence having two or more (e.g., 2, 3, 4, or more) regions of complementarity (e.g., where each region is separated by at least one non-complementary nucleotide) to corresponding co-linear portions of miR-150 (e.g., hsa-miR-150-5p), where the total length of complementarity is, or is at least 5, 6, 7, 8, 9, 10, 11, 12, 13, 14, 15, 16, 17, 18, 19, 20, or 21 nucleotides long. In some embodiments, the inhibitory nucleic acid includes a nucleotide sequence complementary to a portion of miR-150 (e.g., hsa-miR-150-5p) that is about 5-20 nucleotides, about 5-15 nucleotides, or about 5-10 nucleotides long. In some embodiments, the nucleic acid includes one or more miR-150 binding sites. In some embodiments, the nucleic acid includes GGGAG (SEQ ID NO: 19).

In some embodiments, the inhibitory nucleic acid includes a nucleotide sequence complementary to at least a portion of miR-98. In some embodiments, the inhibitory nucleic acid binds that binds to miR-98 inhibits suppression of IL-10 expression by miR-98 in a cell (e.g., a Treg). In some embodiments, miR-98 is human miR-98. In some embodiments, the nucleic acid binds to hsa-miR-98-5p, having a nucleotide sequence of 5′-ugagguaguaaguuguauuguu-3′ (SEQ ID NO: 10). In some embodiments, the inhibitory nucleic acid includes a nucleotide sequence complementary to a portion of miR-98 (e.g., hsa-miR-98-5p) that is, or is at least 5, 6, 7, 8, 9, 10, 11, 12, 13, 14, 15, 16, 17, 18, 19, 20, 21, 22, or 23 nucleotides long. In some embodiments, the inhibitory nucleic acid includes a nucleotide sequence complementary to a contiguous portion of miR-98 (e.g., hsa-miR-98-5p) that is, or is at least 5, 6, 7, 8, 9, 10, 11, 12, 13, 14, 15, 16, 17, 18, 19, 20, 21, 22, or 23 nucleotides long. In some embodiments, the inhibitory nucleic acid includes a nucleotide sequence having two or more (e.g., 2, 3, 4, or more) regions of complementarity (e.g., where each region is separated by at least one non-complementary nucleotide) to miR-98 (e.g., hsa-miR-98-5p), where the total length of complementarity is, or is at least 5, 6, 7, 8, 9, 10, 11, 12, 13, 14, 15, 16, 17, 18, 19, 20, or 21 nucleotides long. In some embodiments, the inhibitory nucleic acid includes a nucleotide sequence having two or more (e.g., 2, 3, 4, or more) regions of complementarity (e.g., where each region is separated by at least one non-complementary nucleotide) to corresponding co-linear portions of miR-98 (e.g., hsa-miR-98-5p), where the total length of complementarity is, or is at least 5, 6, 7, 8, 9, 10, 11, 12, 13, 14, 15, 16, 17, 18, 19, 20, or 21 nucleotides long. In some embodiments, the inhibitory nucleic acid includes a nucleotide sequence complementary to a portion of miR-98 (e.g., hsa-miR-98-5p) that is about 5-20 nucleotides, about 5-15 nucleotides, or about 5-10 nucleotides long. In some embodiments, the nucleic acid includes one or more miR-98 binding sites. In some embodiments, the nucleic acid includes ACAAC (SEQ ID NO: 18).

A nucleic acid of the present disclosure can be single stranded or double stranded (e.g., RNA/DNA hybrid). In some embodiments, the nucleic acid is single stranded. As used herein, “single stranded” denotes that the nucleic acid is composed of one contiguous molecule of the nucleic acid. In some embodiments, the single stranded nucleic acid can have regions of self-complementarity so such that complementary regions within the molecule may hybridize to each other under suitable conditions (e.g., to form a hairpin loop).

An isolated nucleic acid of the present disclosure in some embodiments includes one or more chemically-modified nucleotides, e.g., nucleotides with a modified backbone. In general, the chemical modification(s) is one that substantially preserves or enhances the therapeutic potency of the nucleic acid. Any suitable number of nucleotides of the nucleic acid can be chemically modified. In some embodiments, the nucleic acid includes 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more chemically-modified nucleotides. In some embodiments, the nucleic acid includes 1-5, 1-6, 1-7, 1-8, 1-9, 1-10, 1-15, 1-20, 1-25, 1-30, or 1-35 chemically-modified nucleotides. In some embodiments, the nucleic acid includes 1-10 chemically-modified nucleotides. In some embodiments, the nucleic acid includes 8 chemically-modified nucleotides. In some embodiments, the nucleic acid includes 6 chemically-modified nucleotides.

The chemically modified nucleotides can be distributed along the isolated nucleic acid or the BDSS compound in any suitable manner. In some embodiments, the nucleic acid includes at least one chemically-modified nucleotide within the first half of the nucleic acid, e.g., the 5′ half of the nucleic acid. In some embodiments, the nucleic acid includes at least one chemically-modified nucleotide within the second half of the nucleic acid, e.g., the 3′ half of the nucleic acid. In some embodiments, the nucleic acid includes at least one chemically-modified nucleotide within the first half of the nucleic acid, e.g., the 5′ half of the nucleic acid, and at least one chemically-modified nucleotide within the second half of the nucleic acid, e.g., the 3′ half of the nucleic acid. In some embodiments, the nucleic acid includes one or more chemically-modified nucleotides within 1, 2, 3, 4, 5, 6, 7, 8, 9, 10 or more nucleotides from the 5′ end of the nucleic acid. In some embodiments, the nucleic acid includes one or more chemically-modified nucleotides within 1, 2, 3, 4, 5, 6, 7, 8, 9, 10 or more nucleotides from the 3′ end of the nucleic acid. In some embodiments, no two chemically-modified nucleotides are adjacent each other in the nucleic acid. In some embodiments, the nucleic acid includes 1, 1, 2, 2, 3, 3, 4, 4, 5, 5 chemically-modified nucleotides within 1, 2, 3, 4, 5, 6, 7, 8, 9, 10 nucleotides, respectively, from the 5′ end of the nucleic acid. In some embodiments, the nucleic acid includes 1, 1, 2, 2, 3, 3, 4, 4, 5, 5 chemically-modified nucleotides within 1, 2, 3, 4, 5, 6, 7, 8, 9, 10 nucleotides, respectively, from the 3′ end of the nucleic acid. In some embodiments, the nucleic acid includes the same number of chemically-modified nucleotides in the 5′ half and 3′ half of the nucleic acid. In some embodiments, the nucleic acid includes 3 chemically-modified nucleotides within 5 nucleotides from the 5′ end of the nucleic acid and/or 3 chemically-modified nucleotides within 5 nucleotides from the 3′ end of the nucleic acid.

In some embodiments, the chemically-modified nucleotides are within the nucleotide sequence of (SEQ ID NO: 17), or sequence variant thereof. In some embodiments, the chemically-modified nucleotides are within the nucleotide sequence of UCCCUCAAAGCAACAACCCCC (SEQ ID NO: 11) or sequence variant thereof. In some embodiments, the chemically-modified nucleotides are within the nucleotide sequence of GAGGCUAAGAGGCGGGAGGAU (SEQ ID NO: 12) or sequence variant thereof. In some embodiments, the chemically-modified nucleotides are within the nucleotide sequence of ACUUCCCUCAAAGCAACAACC (SEQ ID NO: 13) or sequence variant thereof. In some embodiments, the chemically-modified nucleotides are within the nucleotide sequence of rUrCrCrCrUrCrArArArGrCrArArCrArArCrCrCrCrC (SEQ ID NO: 14) or sequence variant thereof. In some embodiments, the chemically-modified nucleotides are within the nucleotide sequence of rGrArGrGrCrUrArArGrArGrGrCrGrGrGrArGrGrArU (SEQ ID NO: 15) or sequence variant thereof. In some embodiments, the chemically-modified nucleotides are within the nucleotide sequence of rArCrUrUrCrCrCrUrCrArArArGrCrArArCrArArCrC (SEQ ID NO: 16) or sequence variant thereof. In some embodiments, the nucleic acid includes one or more chemically-modified nucleotides within 1, 2, 3, 4, 5, 6, 7, 8, 9, 10 or more nucleotides from the 5′ end of the nucleotide sequence. In some embodiments, the nucleic acid includes one or more chemically-modified nucleotides within 1, 2, 3, 4, 5, 6, 7, 8, 9, 10 or more nucleotides from the 3′ end of the nucleotide sequence. In some embodiments, no two chemically-modified nucleotides are adjacent each other in the nucleotide sequence. In some embodiments, the nucleic acid includes 1, 1, 2, 2, 3, 3, 4, 4, 5, 5 chemically-modified nucleotides within 1, 2, 3, 4, 5, 6, 7, 8, 9, 10 nucleotides, respectively, from the 5′ end of the nucleotide sequence. In some embodiments, the nucleic acid includes 1, 1, 2, 2, 3, 3, 4, 4, 5, 5 chemically-modified nucleotides within 1, 2, 3, 4, 5, 6, 7, 8, 9, 10 nucleotides, respectively, from the 3′ end of the nucleotide sequence. In some embodiments, the nucleic acid includes the same number of chemically-modified nucleotides in the 5′ half and 3′ half of the nucleotide sequence. In some embodiments, the nucleic acid includes 3 chemically-modified nucleotides within 5 nucleotides from the 5′ end of the nucleotide sequence and/or 3 chemically-modified nucleotides within 5 nucleotides from the 3′ end of the nucleotide sequence. In some embodiments, the nucleic acid includes a different number of chemically-modified nucleotides in the 5′ half and 3′ half of the nucleotide sequence. In some embodiments, the nucleic acid includes a greater number of chemically-modified nucleotides in the 3′ half than in the 5′ half of the nucleotide sequence. In some embodiments, the nucleic acid includes 3 chemically-modified nucleotides within 5 nucleotides from the 5′ end of the nucleotide sequence and/or 3, 4, or 5 chemically-modified nucleotides within 5 nucleotides from the 3′ end of the nucleotide sequence.

In some embodiments, the chemically-modified nucleotide(s) increases in vitro and/or in vivo stability of the nucleic acid. In some embodiments, the chemically-modified nucleotide(s) increases therapeutic potency of the nucleic acid, e.g., for treating an immune-related disorder, e.g., an inflammatory condition, myocarditis, or myocardial infarction.

The isolated nucleic acid or BDSS compound in some embodiments includes one type, or two or more different types of chemically-modified nucleotides. In some embodiments, the chemically-modified nucleotide has a methylene bridge connecting the 2′-O atom and the 4′-C atom of the nucleotide sugar ring to lock the conformation (Locked Nucleic Acid (LNA)). In some embodiments, the isolated nucleic acid includes the nucleotide sequence SEQ ID NO: 17, or a sequence variant thereof, where one or more nucleotides are LNA. In some embodiments, the isolated nucleic acid has the nucleotide sequence SEQ ID NO: 17, or a sequence variant thereof, where one or more positions are LNA. In some embodiments, the chemically-modified nucleotide comprises a modified sugar. In some embodiments, the chemically-modified nucleotide comprises methylation. In some embodiments, the modification is the introduction of a 2′-O-methyl or 2′-O-methoxyethyl group or 2′ fluoride group on the nucleic acid, e.g., to improve nuclease resistance and binding affinity to RNA, e.g., miR. In some embodiments, the chemically-modified nucleotide comprises a 2′-O-methylation.

In some embodiments, the chemically-modified nucleotide includes a modified nucleobase. In some embodiments, modified nucleobases in the nucleic acids are selected from the group consisting of 1-methyl-pseudouridine (m1ψ), 5-methoxy-uridine (mo5U), 5-methyl-cytidine (m5C), pseudouridine (ψ), α-thio-guanosine and α-thio-adenosine. In some embodiments, polynucleotides includes a combination of at least 1 (e.g., 1, 2, 3, 4 or more) of the modified nucleobases. In some embodiments, nucleic acids comprise pseudouridine (ψ), 5-methoxy-uridine (mo5U), pseudouridine (ψ) and/or 5-methyl-cytidine (m5C). In some embodiments, nucleic acids comprise 1-methyl-pseudouridine (m1ψ). In some embodiments, nucleic acids comprise 1-methyl-pseudouridine (m1ψ) and 5-methyl-cytidine (m5C). In some embodiments, nucleic acids comprise methoxy-uridine (mo5U).

In some embodiments, nucleic acids are uniformly modified (e.g., fully modified, modified throughout the entire sequence) for a particular modification. For example, a polynucleotide can be uniformly modified with 1-methyl-pseudouridine (m1ψ), meaning that all uracil residues in the sequence are replaced with 1-methyl-pseudouridine (m1ψ). Similarly, a nucleic acid can be uniformly modified for any type of nucleoside residue present in the sequence by replacement with a modified residue such as those set forth above.

In some embodiments, nucleobases and nucleosides having a modified cytosine include, without limitation, N4-acetyl-cytidine (ac4C), 5-methyl-cytidine (m5C), 5-halo-cytidine (e.g., 5-iodo-cytidine), 5-hydroxymethyl-cytidine (hm5C), 1-methyl-pseudoisocytidine, 2-thio-cytidine (s2C), and 2-thio-5-methyl-cytidine. In some embodiments, a modified nucleobase is a modified uridine. Exemplary nucleobases and In some embodiments, a modified nucleobase is a modified cytosine, nucleosides having a modified uridine include, without limitation, 5-cyano uridine, and 4′-thio uridine. In some embodiments, a modified nucleobase is a modified adenine. Exemplary nucleobases and nucleosides having a modified adenine include, without limitation, 7-deaza-adenine, 1-methyl-adenosine (m1A), 2-methyl-adenine (m2A), and N6-methyl-adenosine (m6A). In some embodiments, a modified nucleobase is a modified guanine. Exemplary nucleobases and nucleosides having a modified guanine include, without limitation, inosine (1), 1-methyl-inosine (m1I), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1G), 8-oxo-guanosine, 7-methyl-8-oxo-guanosine.

In some embodiments, the isolated nucleic acid includes the nucleotide sequence UCCCUCAAAGCAACAACCCCC (SEQ ID NO: 11), or a sequence variant thereof, where one or more positions are chemically modified (e.g., are LNA or are methylated). In some embodiments, the isolated nucleic acid has the nucleotide sequence UCCCUCAAAGCAACAACCCCC (SEQ ID NO: 11), where one or more positions are chemically modified (e.g., are LNA or are methylated).

In some embodiments, the isolated nucleic acid includes the nucleotide sequence GAGGCUAAGAGGCGGGAGGAU (SEQ ID NO: 12), or a sequence variant thereof, where one or more positions are chemically modified (e.g., are LNA or are methylated). In some embodiments, the isolated nucleic acid has the nucleotide sequence GAGGCUAAGAGGCGGGAGGAU (SEQ ID NO: 12), where one or more positions are chemically modified (e.g., are LNA or are methylated).

In some embodiments, the isolated nucleic acid includes the nucleotide sequence ACUUCCCUCAAAGCAACAACC (SEQ ID NO: 13), or a sequence variant thereof, where one or more positions are chemically modified (e.g., are LNA or are methylated). In some embodiments, the isolated nucleic acid has the nucleotide sequence ACUUCCCUCAAAGCAACAACC (SEQ ID NO: 13), where one or more positions are chemically modified (e.g., are LNA or are methylated).

In some embodiments, the BDSS compound includes BDSS-138 (rUrCrCrCrUrCrArArArGrCrArArCrArArCrCrCrCrC) (SEQ ID NO: 14), or a sequence variant thereof, where one or more positions are chemically modified (e.g., are LNA or are methylated). In some embodiments, the BDSS compound is BDSS-138 (rUrCrCrCrUrCrArArArGrCrArArCrArArCrCrCrCrC) (SEQ ID NO: 14), where one or more positions are chemically modified (e.g., are LNA or are methylated).

In some embodiments, the BDSS compound includes BDSS-150 (rGrArGrGrCrUrArArGrArGrGrCrGrGrGrArGrGrArU) (SEQ ID NO: 15), or a sequence variant thereof, where one or more positions are chemically modified (e.g., are LNA or are methylated). In some embodiments, the BDSS compound is BDSS-150 (rGrArGrGrCrUrArArGrArGrGrCrGrGrGrArGrGrArU) (SEQ ID NO: 15), where one or more positions are chemically modified (e.g., are LNA or are methylated).

In some embodiments, the BDSS compound includes BDSS-98 (rArCrUrUrCrCrCrUrCrArArArGrCrArArCrArArCrC) (SEQ ID NO: 16), or a sequence variant thereof, where one or more positions are chemically modified (e.g., are LNA or are methylated). In some embodiments, the BDSS compound is BDSS-98 (rArCrUrUrCrCrCrUrCrArArArGrCrArArCrArArCrC) (SEQ ID NO: 16), where one or more positions are chemically modified (e.g., are LNA or are methylated).

The isolated nucleic acid, in some embodiments, can include any suitable chemical modification. In some embodiments, the chemical modification is a backbone modification, e.g., modification of the sugar/phosphate backbone. In some embodiments, the chemical modification is a backbone sugar modification. In some embodiments, the chemically modified nucleotide includes an LNA. In some embodiments, the chemically modified nucleotide includes methylation. In some embodiments, the chemical modification includes the introduction of a phosphorothioate group as linker between nucleotides. Suitable backbone modifications of the chemically-modified nucleotides include, without limitation, phosphorothioates, phosphotriesters, methyl phosphonates, short chain alkyl or cycloalkyl intersugar linkages or short chain heteroatomic or heterocyclic intersugar linkages. In some embodiments, the chemical modification is a base modification.

The nucleic acids of the present disclosure can be prepared using any suitable option. Suitable options include, without limitation, chemical synthesis, enzymatic production and/or biological production. In some embodiments, the nucleic acids are prepare using chemical synthesis. Any suitable option for chemical synthesis of nucleic acids can be used. Suitable options include, without limitation, phosphodiester, phosphotriester, phosphoramidite, phosphite-triester, and solid phase synthesis approaches. In some embodiments, preparing the nucleic acids includes in vitro transcription. In some embodiments, the nucleic acids are prepared using recombinant DNA technology. In some embodiments, the nucleic acids are prepared by chemically modifying an unmodified nucleic acid having a nucleotide sequence of interest.

### Vectors

Also provided is a vector (e.g., a plasmid), or collection of vectors, that encodes any one or more of the nucleic acids of the present disclosure. In some embodiments, the vector includes a nucleic acid sequence encoding an RNA comprising BCYRN1, having the sequence of SEQ ID NO: 17, or a derivative thereof. In some embodiments, the vector includes a nucleic acid sequence encoding an RNA comprising at least one of the following sequences: UCCCUCAAAGCAACAACCCCC (SEQ ID NO: 11), GAGGCUAAGAGGCGGGAGGAU (SEQ ID NO: 12), and ACUUCCCUCAAAGCAACAACC (SEQ ID NO: 13). A vector may encode any suitable number of RNA transcripts. In some embodiments, a single vector encodes a single RNA transcript (e.g., including any one of SEQ ID NOs: 11-13). In some embodiments, a single vector encodes two or more different RNA transcripts (e.g., each including any one of SEQ ID NOs: 11-13). In some embodiments, RNA comprising each of SEQ ID NOs: 11-13 are encoded in a separate vector (e.g., three separate vectors). In some embodiments, RNA comprising at least one of SEQ ID NOs: 11-13 are each encoded in a single vector (e.g., as one, two or three different transcripts).

In some embodiments, the vector includes one or more regulatory sequences (e.g., promoter, transcriptional terminator, etc.) configured to express the encoded RNA in an environment of interest. In some embodiments, the nucleic acid sequence encoding an RNA comprising BCYRN1, having the sequence of SEQ ID NO: 17, or a derivative thereof is operably linked to the regulatory sequences (e.g., promoter). In some embodiments, the nucleic acid sequence encoding an RNA comprising at least one of the following sequences: UCCCUCAAAGCAACAACCCCC (SEQ ID NO: 11), GAGGCUAAGAGGCGGGAGGAU (SEQ ID NO: 12), and ACUUCCCUCAAAGCAACAACC (SEQ ID NO: 13) is operably linked to the regulatory sequences (e.g., promoter). In some embodiments, the vector includes a CMV promoter. In some embodiments, the promoter is a tissue or cell type-specific promoter. In some embodiments, the promoter is a conditional promoter. In some embodiments the vector is a viral vector, such as, without limitation, a lentiviral vector, adenovirus vector, adeno-associated virus (AAV) vector, herpesviral vector.

### Compositions

Also provided herein are compositions (e.g., therapeutic compositions) that include the nucleic acids or BDSS compounds or inhibitory nucleic acid of the present disclosure.

Provided herein is a therapeutic composition comprising at least one nucleic acid comprising a (e.g., 1, 2, or 3 or more) BDSS (BCYRN1-derived binding sequence), where the BDSS includes a nucleotide sequence as provided by any one of SEQ ID NOs: 11-16, or a sequence at least 90% (e.g., at least 95%, or at least 97%, or about 100%) identical thereto, wherein each of the at least one nucleic acid is no more than 35 nucleotides (e.g., no more than 35, 30, 25, 24, 23, 22, or 21 nucleotides) in length. The composition can include any suitable BDSS (e.g., BDSS-138, BDSS-150, BDSS-98) or BDSS compound, or include any suitable nucleic acid, as described herein. In some embodiments, the composition includes at least two of BDSS-138, BDSS-150, and BDSS-98 (e.g., at least BDSS-138 and BDSS-150, at least BDSS-138 and BDSS-98, or at least BDSS-150 and BDSS-98). In some embodiments, the composition includes at least three BDSS, or BDSS compounds (e.g., BDSS-138, BDSS-150, and BDSS-98).

Also provided is a therapeutic composition comprising at least one nucleic acid comprising a (e.g., 1, 2, or 3 or more) BDSS (BCYRN1-derived binding sequence), where the BDSS includes a nucleotide sequence as provided by any one of SEQ ID NOs: 11-16, or a derivative thereof having 1, 2, 3, 4, or 5 substitutions thereto, for the treatment of a disease of immunity and/or inflammation. In some embodiments, the BDSS or BDSS compound includes at least one of BDSS-138, BDSS-150, and BDSS-98, or a derivative thereof having 1, 2, 3, 4, or 5 substitutions thereto. In some embodiments, the BDSS or BDSS compound includes a nucleotide sequence comprising at least one of SEQ ID NOs: 11-13, or a derivative thereof having 1, 2, 3, 4, or 5 substitutions thereto. In some embodiments, the BDSS or BDSS compound includes a nucleotide sequence comprising at least one of SEQ ID NOs: 11-13, or a derivative thereof having at least 80% sequence identity thereto.

Provided herein is a therapeutic composition comprising at least one inhibitory nucleic acid that each binds to at least one (e.g., 1, 2, or 3) of miR-138, miR-150 and miR-98, wherein the at least one nucleic acid is at most 35 nucleotides long. In some embodiments, the composition includes a first inhibitory nucleic acid that binds to miR-138; a second inhibitory nucleic acid that binds to miR-150; and a third inhibitory nucleic acid that binds to miR-98, wherein each of the first, second and third inhibitory nucleic acids are at most 35 nucleotides long. In some embodiments, the composition includes at least two of the first, second and third inhibitory nucleic acids. In some embodiments, the composition includes the first, second and third inhibitory nucleic acids. In some embodiments, the inhibitory nucleic acid that binds to (e.g., hybridizes with) miR-138 or miR-98 comprises ACAAC (SEQ ID NO: 18). In some embodiments, the inhibitory nucleic acid that binds to (e.g., hybridizes with) miR-150 comprises GGGAG (SEQ ID NO: 19).

Also provided is a therapeutic composition comprising one or more synthetic derivatives of the following long non-coding RNA GGCCGGGCGCGGUGGCUCACGCCUGUAAUCCCAGCUCUCAGGGAGGCUAAGAG GCGGGAGGAUAGCUUGAGCCCAGGAGUUCGAGACCUGCCUGGGCAAUAUAGC GAGACCCCGUUCUCCAGAAAAAGGAAAAAAAAAAACAAAAGACAAAAAAAAA AUAAGCGUAACUUCCCUCAAAGCAACAACCCCCCCCCCCCUUU (SEQ ID NO: 17), wherein the derivative is no more than 35 nucleotides in length, and wherein the synthetic derivative enhances Treg functionality. The synthetic derivatives of the long non-coding RNA can be any suitable derivative that provides for enhanced Treg functionality, e.g., when administered to a subject in need thereof, or when administered to Treg cells in vitro. In some embodiments, the synthetic derivative enhances Treg functionality by enhancing proliferation, migration and/or IL-10 production by the Treg cells.

Also provided is a therapeutic composition comprising a first BDSS (BCYRN1-derived binding sequence) compound as provided by SEQ ID NO: 11, wherein the first BDSS compound is no more than 35 nucleotides in length; a second BDSS compound as provided by SEQ ID NO:12, wherein the second BDSS compound is no more than 35 nucleotides in length; a third BDSS compound as provided by SEQ ID NO: 13, wherein the third BDSS compound is no more than 35 nucleotides in length; and a pharmaceutically acceptable excipient.

In some embodiments, the composition is a pharmaceutical or therapeutic composition. In some embodiments the composition includes pharmaceutically acceptable excipient. In some embodiments, the composition is a cell-free composition, e.g., the composition is substantially free of cells such as CDC. In some embodiments, the composition is an extracellular vesicle-free composition, e.g., the composition is substantially free of extracellular vesicles, such as exosomes. In some embodiments, the composition includes extracellular vesicles that have been combined with the nucleic acids.

Some non-limiting examples of materials which can serve as pharmaceutically-acceptable excipients include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations.

In some embodiments, the composition includes a transfection reagent, e.g., to promote delivery of the nucleic acid to a target cellular target (in vitro or in vivo). Any suitable transfection reagent can be included in the composition. Suitable transfection reagents include, without limitation, a liposome, a lipid nanoparticle (LNP), extracellular vesicle (EV), and a polyethylene glycol (PEG)-cationic lipid complex (PCLC). In some embodiments, the transfection reagent includes a lipid (e.g., a liposome-forming lipid), or a PEGylated lipid. In some embodiments, the lipid is a cationic lipid, as provided herein. In some embodiments, the transfection reagent includes DharmaFECT® or Lipofectamine®. In some embodiments, the nucleic acid of the present disclosure is formulated with the transfection reagent in the composition so as to promote cellular uptake and/or pharmacokinetics of the nucleic acid. In some embodiments, the transfection reagent is a LNP.

Liposomes are artificially-prepared vesicles which may primarily be composed of a lipid bilayer and may be used as a delivery vehicle for the administration of pharmaceutical formulations. Liposomes can be of different sizes such as, but not limited to, a multilamellar vesicle (MLV), which may be hundreds of nanometers in diameter and may contain a series of concentric bilayers separated by narrow aqueous compartments, a small unicellular vesicle (SUV), which may be smaller than 50 nm in diameter, and a large unilamellar vesicle (LUV), which may be between 50 and 500 nm in diameter. Liposome design may include, without limitation, opsonins or ligands in order to improve the attachment of liposomes to target tissue/cells, or to activate events such as, but not limited to, endocytosis. Liposomes may contain a low or a high pH in order to improve the delivery of the cargo, e.g., a nucleic acid of the present disclosure.

In some embodiments, the composition includes, without limitation, liposomes such as those formed from 1,2-dioleyloxy-N,N-dimethylaminopropane (DODMA) liposomes, DiLa2 liposomes from Marina Biotech (Bothell, Wash.), 1,2-dilinoleyloxy-3-dimethylaminopropane (Dlin-DMA), 2,2-dilinoleyl-4-(2-dimethylaminoethyl)-[1,3]-dioxolane (Dlin-KC2-DMA), and MC3 and liposomes such as, but not limited to, DOXIL® from Janssen Biotech, Inc. (Horsham, Pa.).

In some embodiments, the composition includes a cationic lipid. Any suitable cationic lipid may be used in the present compositions. Suitable cationic lipids include, without limitation, Dlin-DMA, Dlin-D-DMA, Dlin-MC3-DMA, Dlin-KC2-DMA, DODMA and amino alcohol lipids. In some embodiments, the composition includes a cationic lipid complex, e.g., a polyethylene glycol (PEG)-cationic lipid complex (PCLC). In some embodiments, the cationic lipid is PEGylated, e.g., 2 kDa PEG (“PEG2000”). Any suitable option can be used to PEGylate the cationic lipid. In some embodiments, PCLC is formed by exposing a mixture of PEG and the cationic lipid to one or more freeze/thaw cycles, e.g., 1, 2, 3, 4, 5 or more freeze/thaw cycles. In some embodiments, a freeze/thaw cycle includes freezing the mixture with liquid nitrogen (e.g., around −190° C.) for about 5 minutes, and thawing at about 60° C. for about 5 minutes. A nucleic acid of the present disclosure can be mixed with the PCLC to generate a complex of the nucleic acid and the PCLC. In some embodiments, the composition includes LNPs.

In some embodiments, the composition includes extracellular vesicles (EV), e.g., exosomes. The extracellular vesicles (EV) can be those from any suitable source, e.g., EV derived from cardiosphere-derived cells (CDC), or from fibroblasts. Suitable EV, such as CDC-derived EV, are provided in, e.g., U.S. Application Publication Nos. 20080267921, 20160158291 and 20160160181; Smith et al., Circulation. 2007. 115:896-908; Aminzadeh, M. A. et al. Stem Cell Reports 10, 942-955 (2018); and Ibrahim et al., Stem Cell Reports. 2014 May 8; 2(5):606-19, Ibrahim, A. G. et al. Nanomedicine 33, 102347 (2020), each of which is incorporated by reference in its entirety. In some embodiments, the Evs are those isolated from serum-free media conditioned by human CDCs in culture. In some embodiments, the composition includes EV and liposomes and/or PCLC as transfection reagents. In some embodiments, the composition is substantially free of CDC-derived EV.

EVs, e.g., exosomes, disclosed herein can vary in size, depending on the embodiment. Depending on the embodiment, the size of the EVs ranges in diameter from about 15 nm to about 95 nm in diameter, including about 15 nm to about 20 nm, about 20 nm to about 30 nm, about 30 nm to about 40 nm, about 40 nm to about 50 nm, about 50 nm to about 60 nm, about 60 nm to about 70 nm, about 70 nm to about 80 nm, about 80 nm to about 90 nm, about 90 nm to about 95 nm, and overlapping ranges thereof. In several embodiments, EVs are larger (e.g., those ranging from about 140 to about 210 nm, including about 140 nm to about 150 nm, about 150 nm to about 160 run, about 160 nm to about 170 nm, about 170 nm to about 180 nm, about 180 nm to about 190 nm, 190 nm to about 200 nm, about 200 nm to about 210 nm, and overlapping ranges thereof). In some embodiments, the EV diameter is in a range of about 15 nm to about 200 nm in diameter, including about 15 nm to about 20 nm, about 20 nm to about 30 nm, about 30 nm to about 40 nm, about 40 nm to about 50 nm, about 50 nm to about 60 nm, about 60 nm to about 70 nm, about 70 nm to about 80 nm, about 80 nm to about 90 nm, about 90 nm to about 100 nm, about 100 nm to about 110 nm, about 110 nm to about 120 nm, about 120 nm to about 130 nm, about 130 nm to about 140 nm, about 140 nm to about 150 nm, about 150 nm to about 160 nm, about 160 nm to about 170 nm, about 170 nm to about 180 nm, about 180 nm to about 190 nm, about 190 nm to about 200 nm, and overlapping ranges thereof. In some embodiments, the EVs that are generated from the original cellular body are 100, 200, 300, 400, 500, 600, 700, 800, 900, 1,000, 2,000, 5,000, or 10,000 times smaller in at least one dimension (e.g., diameter) than the original cellular body.

The composition containing the EV and nucleic acid of the present disclosure can be prepared using any suitable option. In some embodiments, loading the nucleic acid into the EV includes: formulating the nucleic acid with liposomes and/or PCLC, e.g., as provided above, to generate a nucleic acid-liposome mixture; combining the nucleic acid-liposome mixture with the EV; and enriching for EV associated with exosome markers to generate a population of EV enriched for the nucleic acid. Combining the nucleic acid-liposome mixture with the EV can be done using any suitable option. In some embodiments, the nucleic acid-liposome mixture is combined with the EV at 37° C. with shaking for about 30 minutes or more. Enriching to generate a population of EV enriched for the nucleic acid can be done using any suitable option. In some embodiments, enriching for EV associated with exosome markers includes immunoprecipitating EV associated with exosome markers using antibodies specific to an exosome marker. In some embodiments, the exosome marker is one or more of CD9, CD63 and CD81. In some embodiments, enriching for EV associated with exosome markers includes immunoprecipitating EV associated with all the exosome markers, CD9, CD63 and CD81. In some embodiments, the size distribution of the population of EV enriched for the nucleic acid is substantially unimodal. In some embodiments, at least 80%, 85%, 90%, 95%, 97%, 99% of the population has a diameter under a single peak in the size distribution. In some embodiments, the population of EV enriched for the nucleic acid has an average diameter of about 50-180 nm, e.g., 60-170 nm, 70-160 nm, 80-150 nm, 90-140 nm, 100-130 nm, or about 110-130 nm.

In some embodiments, the composition includes casein, e.g., a casein micelle. In some embodiments, the composition includes chitosan. In some embodiments, the composition includes casein and chitosan, e.g., a casein-chitosan micelle. In some embodiments, the composition includes a casein-chitosan complex. In some embodiments, the isolated nucleic acid in the composition is encapsulated in a casein-chitosan complex. In some embodiments, the composition includes one or more of phosphoproteins: alpha s1 casein, alpha s2 casein, beta casein, and kappa casein. In some embodiments, the composition includes two or more, three or more, or all four phosphoproteins: alpha s1 casein, alpha s2 casein, beta casein, and kappa casein. The phosphoproteins may be present in the composition at any suitable concentration (relative to each other, and relative to the total volume of the composition), and in some embodiments, is present in an amount suitable for forming casein micelles. In some embodiments, the casein phosphoproteins are collectively present in the composition at about 5-10% (weight by volume). In some embodiments, the casein phosphoproteins are collectively present in the composition at about 8% (weight by volume). In some embodiments, the casein phosphoproteins are collectively present in the composition at about 5% (weight by volume). The casein phosphoproteins can be those from any suitable animal, e.g., mammal such as, but not limited to, human, non-human primate, cow, pig, horse, camel, goat, and sheep. In some embodiments, the casein phosphoproteins are bovine alpha s1 casein, alpha s2 casein, beta casein, and kappa casein. Suitable casein formulations with EV are provided in, e.g., Aminzadeh et al., J Extracell Vesicles. 2021 January; 10(3):e12045, the entirety of which is incorporated herein by reference. In some embodiments, a composition, e.g., pharmaceutical composition, of the present disclosure formulated with casein, as provided herein, is suitable for oral administration to the subject. Without being bound by theory, the casein phosphoproteins in the composition are thought to increase the bioavailability of orally administered EV and/or liposomes and their cargo, e.g., the nucleic acid of the present disclosure.

In several embodiments, the composition comprises at least two phosphoproteins selected from alpha s1 casein, alpha s2 casein, beta casein, and kappa casein, where the phosphoproteins are present in an amount between about 5% to about 10% (weight by volume) of the composition, in a physiologically compatible excipient. In several embodiments, the composition includes the alpha s1 casein in an amount between about 0% to about 50% (e.g., about 10% to about 45%, about 20% to about 40%, about 25% to about 40%, about including 30% to about 40%) (by weight), the alpha s2 casein in an amount between about 0% to about 20% (e.g., about 5% to about 15%, about 7% to about 12%, including about 8% to about 12%) (by weight), the beta casein in an amount between about 0% to about 50% (e.g., about 10% to about 45%, about 20% to about 40%, about 25% to about 40%, about including 30% to about 40%) (by weight), and the kappa casein in an amount between about 0% to about 20% (e.g., about 5% to about 18%, about 8% to about 18%, including about 10% to about 15%) (by weight) of the phosphoprotein mass in the composition. The present compositions can provide for enhanced oral bioavailability of therapeutic nucleic acids, such as BCYRN1, or a derivative(s) thereof (e.g., one or more BDSS (BCYRN1-derived binding sequence), such as BDSS-138, BDSS-150, and/or BDSS-98).

In several embodiments, the formulations provided for herein include lipid-bound vesicles, e.g., micelles or liposomes, and can therefore include any suitable number of particles. In some embodiments, the amount of micelles (e.g., casein-chitosan coated micelles) is in a range of about 10⁶ to about 10¹⁰ particles, e.g., about 2×10⁶ to about 10¹⁰ particles, about 5×10⁶ to about 10¹⁰ particles, about 10⁷ to about 5×10⁹ particles, about 2×10⁷ to about 5×10⁹ particles, about 5×10⁷ to about 5×10⁹ particles, including about 1×10⁸ to about 2×10⁹ particles. In some embodiments, the amount of micelles (e.g., casein-chitin coated micelles) in the population is about 10⁶, about 2×10⁶, about 5×10⁶, about 10⁷, about 2×10⁷, about 5×10⁷, about 10⁸, about 2×10⁸, about 5×10⁸, about 10⁹, about 2×10⁹, about 5×10⁹, or about 10¹⁰ particles, or an amount in between any two of the preceding values.

In several embodiments, the composition comprises casein-chitosan coated lipid micelles, where the casein phosphoproteins are present in the composition in suitable amounts (e.g., suitable total amount of phosphoprotein mass in the composition, suitable proportions of phosphoproteins relative to each other). In some embodiments, the composition includes two, three, or all four phosphoproteins selected from alpha s1 casein, alpha s2 casein, beta casein, and kappa casein. In some embodiments, the amount of a phosphoprotein in the composition depends on the amount of one or more other phosphoprotein present in the composition.

In some embodiments, alpha s1 casein is a phosphoprotein associated with the gene name CSN1S1. The alpha s1 casein can be a CSN1S1 phosphoprotein from any suitable mammal. In some embodiments, the alpha s1 casein is bovine (Gene ID: 282208), porcine (Gene ID: 445514), equine (Gene ID: 100033982), ovine (Gene ID: 443382), caprine (Gene ID: 100750242), cameline (Gene ID: 105090954), or human (Gene ID: 1446). In some embodiments, the alpha s1 casein is a non-human alpha s1 casein. In some embodiments, the alpha s1 casein is a polypeptide having an amino acid sequence at least 80%, at least 85%, at least 90%, at least 95%, at least 98%, or about 100% identical to the sequence set forth in SEQ ID NO: 1.

In some embodiments, the composition includes any suitable amount of alpha s1 casein. In some embodiments, the composition includes the alpha s1 casein in an amount, by weight, between about 0% to about 50%, e.g., between about 5% to about 50%, between about 10% to about 50%, between about 15% to about 45%, between about 20% to about 45%, including between about 25% to about 40%, of the phosphoprotein mass in the composition. In some embodiments, the composition includes the alpha s1 casein in an amount, by weight, of about 0%, 5%, 10%, 15%, 20%, 25%, 26%, 27%, 28%, 29%, 30%, 31%, 32%, 33%, 34%, 35%, 36%, 37%, 38%, 39%, 40%, or an amount within a range defined by any two of the preceding values.

In some embodiments, the alpha s2 casein is a phosphoprotein associated with the gene name CSN1S2. The alpha s2 casein can be a CSN1S2 phosphoprotein from any suitable mammal. In some embodiments, the alpha s2 casein is bovine (Gene ID: 282209), porcine (Gene ID: 445515), equine (Gene ID: 100327035), ovine (Gene ID: 443383), caprine (Gene ID: 100861229), or cameline (Gene ID: 105090951). In some embodiments, the alpha s2 casein is a polypeptide having an amino acid sequence at least 80%, at least 85%, at least 90%, at least 95%, at least 98%, or about 100% identical to the sequence set forth in SEQ ID NO: 12.

The composition can include any suitable amount of alpha s2 casein. In some embodiments, the composition includes the alpha s2 casein in an amount, by weight, between about 0% to about 20%, e.g., between about 2% to about 18%, between about 3% to about 18%, between about 4% to about 17%, between about 5% to about 16%, including between about 5% to about 15%, of the phosphoprotein mass in the composition. In some embodiments, the composition includes the alpha s2 casein in an amount, by weight, of about 0%, 1%, 2%. 3%, 4%, 5%, 6%, 7%, 8%, 9%, 10%, 11%, 12%, 13%, 14%, 15%, 18%, 20%, or an amount within a range defined by any two of the preceding values.

In some embodiments, the beta casein is a phosphoprotein associated with the gene name CSN2. The beta casein can be a CSN2 phosphoprotein from any suitable mammal. In some embodiments, the beta casein is bovine (Gene ID: 281099), porcine (Gene ID: 404088), equine (Gene ID: 100033903), ovine (Gene ID: 443391), caprine (Gene ID: 100860784), cameline (Gene ID: 105080412), or human (Gene ID: 1447). In some embodiments, the beta casein is a non-human beta casein. In some embodiments, the beta casein is a polypeptide having an amino acid sequence at least 80%, at least 85%, at least 90%, at least 95%, at least 98%, or about 100% identical to the sequence set forth in SEQ ID NO: 3 or 4.

The composition can include any suitable amount of beta casein. In some embodiments, the composition includes the beta casein in an amount, by weight, between about 0% to about 50%, e.g., between about 5% to about 50%, between about 10% to about 50%, between about 15% to about 45%, between about 20% to about 45%, including between about 25% to about 40%, of the phosphoprotein mass in the composition. In some embodiments, the composition includes the beta casein in an amount, by weight, of about 0%, 5%, 10%, 15%, 20%, 25%, 26%, 27%, 28%, 29%, 30%, 31%, 32%, 33%, 34%, 35%, 36%, 37%, 38%, 39%, 40%, or an amount within a range defined by any two of the preceding values.

In some embodiments, the kappa casein is a phosphoprotein associated with the gene name CSN3. The beta casein can be a CSN3 phosphoprotein from any suitable mammal. In some embodiments, the kappa casein is bovine (Gene ID: 281728), porcine (Gene ID: 445511), equine (Gene ID: 100033983), ovine (Gene ID: 443394), caprine (Gene ID: 100861231), cameline (Gene IDs: 105080408 or 105090949), or human (Gene ID: 1448). In some embodiments, the kappa casein is a non-human kappa casein. In some embodiments, the kappa casein is a polypeptide having an amino acid sequence at least 80%, at least 85%, at least 90%, at least 95%, at least 98%, or about 100% identical to the sequence set forth in SEQ ID NO: 5.

The composition can include any suitable amount of kappa casein. In some embodiments, the composition includes the kappa casein in an amount, by weight, between about 0% to about 20%, e.g., between about 2% to about 18%, between about 3% to about 18%, between about 4% to about 17%, between about 5% to about 16%, including between about 5% to about 15%, of the phosphoprotein mass in the composition. In some embodiments, the composition includes the kappa casein in an amount, by weight, of about 0%, 1%, 2%. 3%, 4%, 5%, 6%, 7%, 8%, 9%, 10%, 11%, 12%, 13%, 14%, 15%, 18%, 20%, or an amount within a range defined by any two of the preceding values.

Combinations of caseins from different species are used, in some embodiments. For example, in several embodiments, one or more human casein is used in combination with one or more bovine casein. Ratios of caseins are used in some embodiments, for example a 3:1:3:1 ratio of alpha S1 casein:alpha s2 casein:beta casein:kappa casein. Different ratios may be used in some embodiments, for example 4:1:4:1, 2:1:2:1, or 1:1:1:1. Ratios may also be used between any two given caseins in a composition, ranging from 1:1, 2:1, 3:1, 4:1, 5:1, 10:1, 1:5, 1:4, 1:3, 1:2, etc.

Any suitable total amount of the phosphoproteins may be present in the composition. In some embodiments, the phosphoproteins are present in an amount between 5% to about 10%, e.g., about 6% to about 10%, about 6% to about 9%, including about 6% to about 8%, (weight by volume) of the composition. In some embodiments, the phosphoproteins are present in an amount of about 5%, 6%, 7%, 8%, 9%, 10%, or an amount within a range defined by any two of the preceding values, (weight by volume) of the composition.

In some embodiments, one or more of the casein phosphoproteins are non-human casein phosphoproteins. In some embodiments, the exosomes and at least one of the casein phosphoproteins are from different species. In some embodiments, the exosomes are human exosomes, and one or more of the casein phosphoproteins are non-human casein phosphoproteins. In some embodiments, the exosomes are human exosomes, and one or more of the casein phosphoproteins are bovine (or ovine, porcine, caprine, cameline, or equine) casein phosphoproteins.

In some embodiments, the composition includes micellar structures formed by at least a portion of the casein phosphoproteins. In some embodiments, the casein micelles are substantially spherical. In some embodiments, a casein micelle in the composition has an average diameter (as measured per micelle) of about 40 nm, about 50 nm, about 60 nm, about 70 nm, about 80 nm, about 90 nm, about 100 nm, about 110 nm, about 120 nm, about 130 nm, about 150 nm, about 200 nm, about 250 nm, about 300 nm, about 350 nm, about 400 nm, about 450 nm, about 500 nm or more, or an average diameter within a range defined by any two of the preceding values. In some embodiments, a casein micelle in the composition has an average diameter (as measured per micelle) in a range from about 40 nm to about 500 nm, e.g., from about 40 nm to about 400 nm, from about 50 nm to about 300 nm, from about 60 nm to about 250 nm, from about 70 nm to about 250 nm, from about 80 nm to about 200 nm, including from about 90 nm to about 150 nm. The casein micelles of the present composition are generally not precipitated or in gel form.

In some embodiments, the composition includes one or more colloidal minerals (e.g., minerals in suspension). In several embodiments, a complex (e.g., two or more) minerals are used as a colloidal mineral complex. The colloidal mineral complex can include any suitable mineral compounds and/or their salts. In some embodiments, the colloidal mineral complex includes, without limitation, one or more of calcium, magnesium, inorganic phosphate, citrate, sodium, potassium, and chloride, or their respective salts. In some embodiments, the colloidal mineral complex is present in an amount between about 2% and about 15%, e.g., about 2% to about 12%, about 5% to about 10%, about 5% to about 9%, including about 6% to about 9% (by weight) of the phosphoprotein mass in the composition. In some embodiments, the colloidal mineral complex is present in an amount of about 2%, 3%, 4%, 5%, 6%, 7%, 8%, 9%, 10%, 11%, 12%, 13%, 14%, 15% or an amount within a range defined by any two of the preceding percentages.

In some embodiments, the composition is in a parenteral dose form. In some embodiments, the parenteral dosage form is sterile or capable of being sterilized before administering to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration to a subject. Suitable excipients that can be used to provide parenteral dosage forms of the nucleic acid include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

Also provided herein is a regulatory T cell (Treg cell) that includes a nucleic acid of the present disclosure. In some embodiments, the Treg cell is a CD4+Foxp3+Treg cell. In some embodiments, the Treg cell is a human Treg cell. In some embodiments, the Treg is an induced human Treg. In some embodiments, a Treg cell that has been exposed to the nucleic acid increases anti-inflammatory activity compared to a suitable control, e.g., a Treg that has not been exposed to the nucleic acid, or a Treg that has been exposed to a control nucleic acid. In some embodiments, the Treg is derived from a patient's peripheral blood. In some embodiments, the Treg has increased expression of one or more of IL-10, ATG-7, and CCR6. In some embodiments, the Treg has increased mRNA expression of one or more of IL-10, ATG-7, and CCR6, compared to a suitable control, e.g., a Treg that has not been contacted with the nucleic acid. In some embodiments, the Treg has increased mRNA expression of one or more of IL-10, ATG-7, and CCR6, compared to a suitable control, e.g., a Treg that has not been contacted with the nucleic acid. In some embodiments, mRNA expression of one or more of IL-10, ATG-7, and CCR6 in the Treg having the nucleic acid is each independently increased by at least 1.5 fold, 2 fold, 4 fold, 6 fold, 8 fold, 10 fold, 15 fold, 20 fold, 30 fold, 50 fold, 100 fold, 200 fold, 300 fold, 400 fold 500 fold, 1,000 fold, or more, or by a fold amount in a range defined by any two of the preceding values, compared to a suitable control, e.g., a Treg that has not been contacted with the nucleic acid. In some embodiments, the Treg has increased protein expression of one or more of IL-10, ATG-7, and CCR6, compared to a suitable control, e.g., a Treg that has not been contacted with the nucleic acid. In some embodiments, protein expression of one or more of IL-10, ATG-7, and CCR6 in the Treg having the nucleic acid is each independently increased by at least 1.5 fold, 2 fold, 4 fold, 6 fold, 8 fold, 10 fold, 15 fold, 20 fold, 30 fold, 50 fold, 100 fold, 200 fold, or more, or by a fold amount in a range defined by any two of the preceding values, compared to a suitable control, e.g., a Treg that has not been contacted with the nucleic acid. In some embodiments, the Treg has increased secretion of IL-10 compared to a suitable control, e.g., a Treg that has not been contacted with the nucleic acid. In some embodiments, IL-10 secretion in the Treg having the nucleic acid is increased by at least about 1.2 fold, 1.5 fold, 2 fold, 2.5 fold, 3 fold, 4 fold, 5 fold, 6 fold, 8 fold, 10 fold, 15 fold, 20 fold, 50 fold, or more, or by a fold amount in a range defined by any two of the preceding values, compared to a suitable control, e.g., a Treg that has not been contacted with the nucleic acid. In some embodiments, the Treg has increased migration compared to a suitable control, e.g., a Treg that has not been contacted with the nucleic acid. In some embodiments, migration of the Treg having the nucleic acid is increased by at least about 1.2 fold, 1.5 fold, 2 fold, 2.5 fold, 3 fold, 4 fold, 5 fold, 6 fold, 8 fold, 10 fold, 15 fold, 20 fold, 50 fold, or more, or by a fold amount in a range defined by any two of the preceding values, compared to a suitable control, e.g., a Treg that has not been contacted with the nucleic acid. In some embodiments, the Treg has increased proliferation compared to a suitable control, e.g., a Treg that has not been contacted with the nucleic acid. In some embodiments, proliferation in the Treg having the nucleic acid is increased by at least about 1.2 fold, 1.5 fold, 2 fold, 2.5 fold, 3 fold, 4 fold, 5 fold, 6 fold, 8 fold, 10 fold, 15 fold, 20 fold, 50 fold, or more, or by a fold amount in a range defined by any two of the preceding values, compared to a suitable control, e.g., a Treg that has not been contacted with the nucleic acid. In some embodiments, the Treg is in culture. In some embodiments, the Treg is in a subject, e.g., in peripheral blood, bone marrow, and/or at a site of tissue injury. In some embodiments, the Treg is autologous to a subject.

Also provided are cells, e.g., regulatory T cells or cardiosphere-derived cells (CDC), genetically modified to express at least one of the nucleic acids described herein. In some embodiments, the cells are genetically modified with a vector encoding one or more nucleic acids of the present disclosure. In some embodiments, the genetically modified cell overexpresses at least one of the BDSS compounds. In some embodiments, the genetically modified cell is a genetically modified CDC, and the cell produces extracellular vesicles (e.g., exosomes) that are enriched in at least one of the BDSS compounds, compared to a CDC that is not genetically modified, or is genetically modified with a control vector.

### Methods

Provided herein are methods of treating a subject in need thereof using the nucleic acids of the present disclosure (also referred to herein as “treatment methods”). Conditions that may be treated by the treatment methods include, without limitation, immune-related disorders, heart conditions and inflammatory conditions. In some embodiments, the conditions include, without limitation, myocarditis myocardial infarction, cardiac disorders, myocardial alterations, inflammatory disease, autoimmune diseases, viral infection, sepsis or wound healing. In some embodiments, conditions treated by the treatment methods include, without limitation, conditions associated with inflammation. In some embodiments, a subject treated by administering the nucleic acids of the present disclosure, according to the treatment methods herein, are in need of treatment for conditions associated with inflammation. The conditions associated with inflammation can include, without limitation, inflammation of the heart, skeletal muscle, or skin. In some embodiments, the conditions associated with inflammation includes aging. In some embodiments, the conditions treated by the present treatment methods are a symptom and/or sequelae of an infection. In some embodiments, the infection is a viral infection, e.g., a respiratory virus infection, such as COVID-19, infections due to other coronaviruses, or other viral pathogens (e.g., flu, H1N1, Hepatitis C, HIV, etc.).

In some embodiments, a treatment method includes a method of treating an immune-related disorder, the method including administering to a subject in need of treating an immune-related disorder a therapeutically effective amount of the nucleic acid (or the composition containing the nucleic acid) of the present disclosure. The immune-related disorder can be, without limitation, a cardiac muscle disorder, autoimmune disease, or transplant rejection. In some embodiments, the immune-related disorder includes myocarditis or myocardial infarction. In some embodiments, the subject has muscular myocarditis or has suffered a myocardial infarction, or is at risk of developing myocarditis or myocardial infarction.

In some embodiments, a treatment method includes a method of treating a heart condition or symptom thereof, the method including administering to a subject in need of treating a heart condition or symptom thereof a therapeutically effective amount of the nucleic acid (or the composition containing the nucleic acid) of the present disclosure. In some embodiments, the subject is a human subject. In some embodiments, the subject is a non-human subject, e.g., a non-human mammal.

A variety of heart conditions may be treated by the present method. In some embodiments, the heart condition includes a symptom and/or sequelae of heart failure, ischemic heart disease, or myocardial infarction. In some embodiments, the heart condition includes myocarditis, or hypertrophic cardiomyopathy. In some embodiments, the heart condition includes heart failure with preserved ejection fraction (HFpEF).

In some embodiments, the subject is at risk of developing the heart condition. In some embodiments, the subject is at risk of developing the heart condition based on one or more of the subject's family history, genetic predisposition, life style, and medical history. In some embodiments, the subject has a mutation in cardiac troponin I that predisposes the subject to developing hypertrophic cardiomyopathy (HCM). In some embodiments, the subject has one or more comorbidities for the heart condition. In some embodiments, the one or more comorbidities includes obesity and hypertension. In some embodiments, the subject has, or is diagnosed with, the heart condition.

In some embodiments, the subject exhibits one or more of: hypertension, elevated E/e′ ratio, cardiac hypertrophy, myocardial fibrosis, obesity, wasting, reduced endurance, and elevated systemic inflammatory markers. In some embodiment, the subject has hypertension, and administering the therapeutically effective amount of the nucleic acid (or composition thereof) reduces the subject's blood pressure. In some embodiments, a subject having hypertension has a resting blood pressure of over 130/90 mmHg. In some embodiments, a subject having hypertension has a resting blood pressure of over 140/90 mmHg. In some embodiment, administering the therapeutically effective amount of the nucleic acid (or composition thereof) reduces the subject's systolic blood pressure or diastolic blood pressure. In some embodiment, the subject's blood pressure (systolic or diastolic blood pressure) is reduced at least to a level that is deemed no longer to be hypertensive after administering the therapeutically effective amount of the nucleic acid (or composition thereof).

In some embodiment, the subject has an elevated E/e′ ratio, and administering the therapeutically effective amount of the nucleic acid (or composition thereof) reduces the E/e′ ratio. In some embodiment, the subject's E/e′ ratio is reduced at least to a level that is deemed no longer to be clinically relevant after administering the therapeutically effective amount of the nucleic acid (or composition thereof).

In some embodiment, the subject has cardiac hypertrophy, and administering the therapeutically effective amount of the nucleic acid (or composition thereof) reduces cardiac hypertrophy. Cardiac hypertrophy can be measured using any suitable option. In some embodiments, cardiac hypertrophy is measured using echocardiography. In some embodiments, a subject having cardiac hypertrophy has an increased diastolic interventricular septal wall diameter (IVSd) and/or left ventricular posterior wall diameter (LVPWd), as measured by echocardiography, and administering the therapeutically effective amount of the nucleic acid (or composition thereof) reduces the IVSd and/or LVPWd. In some embodiment, the subject's IVSd or LVPWd is reduced at least to a level that is deemed no longer to be hypertrophic after administering the therapeutically effective amount of the nucleic acid (or composition thereof).

In some embodiments, the subject has inflammation associated with an autoimmune condition. Non-limiting examples of autoimmune diseases or disorders include autoimmune myocarditis, conditions involving infiltration of T cells and chronic inflammatory responses, arthritis (rheumatoid arthritis, juvenile rheumatoid arthritis, psoriatic arthritis), multiple sclerosis, pemphigus, and type 1 diabetes (also referred to as insulin-dependent diabetes mellitus (IDDM)).

In some embodiment, the subject exhibits wasting or weight loss, and administering the therapeutically effective amount of the nucleic acid (or composition thereof) retards or prevents the wasting. In some embodiment, the subject's body weight recovers to, or is maintained at substantially the pre-treatment level after administering the therapeutically effective amount of the nucleic acid (or composition thereof).

In some embodiment, the subject exhibits reduced endurance, e.g., exercise endurance, and administering the therapeutically effective amount of the nucleic acid (or composition thereof) retards or prevents the decline in endurance. In some embodiment, the subject's exercise endurance recovers to, or is maintained at substantially the pre-treatment level after administering the therapeutically effective amount of the nucleic acid (or composition thereof). In some embodiments, the improvement in endurance after administering the therapeutically effective amount of the nucleic acid (or composition thereof) is sustained over the duration of treatment. In some embodiments, the improvement in endurance after administering the therapeutically effective amount of the nucleic acid (or composition thereof) is sustained across multiple doses of administration.

In some embodiments, the subject exhibits elevated levels of systemic inflammatory markers, e.g., in the peripheral blood. In some embodiments, the systemic inflammatory marker includes one or more of IL-6 and brain natriuretic peptide (BNP). In some embodiment, the subject's systemic inflammatory marker is reduced at least to a level that is deemed no longer to be elevated after administering the therapeutically effective amount of the nucleic acid (or composition thereof).

In some embodiments, where the subject is obese, the therapeutic effect of administering the nucleic acid is independent of the subject's obesity. In some embodiments, administering the nucleic acid (or composition thereof) does not affect the subject's weight.

In some embodiments, the subject exhibits reduced skeletal muscle function, e.g., the amount of force or torque exerted by a skeletal muscle group. In some embodiments, the subject exhibits reduced skeletal muscle function and administering the therapeutically effective amount of the nucleic acid (or composition thereof) retards the development of reduced skeletal muscle function, prevents deterioration of skeletal muscle function, or enhances skeletal muscle function. In some embodiment, the subject's skeletal muscle function recovers to, or is maintained at substantially the pre-treatment level after administering the therapeutically effective amount of the nucleic acid (or composition thereof).

In some embodiments, any of the therapeutic effects of administering the therapeutically effective amount of the nucleic acid (or composition thereof) herein is sustained over the duration of treatment. Is sustained across multiple doses of administration is sustained across multiple doses of administration. In some embodiments, any of the therapeutic effects of administering the therapeutically effective amount of the nucleic acid (or composition thereof) herein is not transient over the duration of treatment.

In some embodiments, a treatment method of the present disclosure treats any one or more of a variety of inflammatory conditions. In some embodiments, the inflammatory condition is a chronic condition. In some embodiments, the inflammatory condition is one that is responsive to the anti-inflammatory effect of IL-10. In some embodiments, the inflammatory condition includes an autoimmune disease, graft-versus-host disease (GVHD) or an immune response to an organ transplant. In some embodiments, the inflammatory condition includes viral infection, sepsis, arthritis (rheumatoid arthritis, juvenile rheumatoid arthritis, psoriatic arthritis), multiple sclerosis, pemphigus, and type 1 diabetes (also referred to as insulin-dependent diabetes mellitus (IDDM)). In some embodiments, the inflammatory condition includes Behget's disease, polymyositis/dermatomyositis, autoimmune cytopenias, autoimmune myocarditis, primary liver cirrhosis, Goodpasture's syndrome, autoimmune meningitis, Sjögren's syndrome, systemic lupus erythematosus, Addison's disease, alopecia greata, ankylosing spondylitis, autoimmune hepatitis, autoimmune mumps, Crohn's disease, insulin-dependent diabetes mellitus, dystrophic epidermolysis bullosa, epididymitis, glomerulonephritis, Graves' disease, Guillain-Barre syndrome, Hashimoto's disease, hemolytic anemia, multiple sclerosis, myasthenia gravis, pemphigus vulgaris, psoriasis, rheumatic fever, rheumatoid arthritis, sarcoidosis, scleroma, spondyloarthropathy, thyroiditis, vasculitis, vitiligo, myxedema, pernicious anemia and ulcerative colitis. In some embodiments, the inflammation is related to a bone marrow transplantation. In some embodiments, the inflammation is related to allograft rejection following tissue transplantation. In some embodiments, the autoimmune disease is a cardiac autoimmune disease, e.g., autoimmune myocarditis. In some embodiments, the autoimmune disease is scleroderma or systemic sclerosis.

In some embodiments, a treatment method of the present disclosure treats symptoms and/or sequelae of any one or more of a variety of infectious diseases. In some embodiments, a heart condition or inflammatory condition treated by the nucleic acids of the present disclosure includes a symptom and/or sequelae of an infectious disease. In some embodiments, the infectious disease is associated with myocardial injury. In some embodiments, the heart condition includes acute myocarditis associated with the infectious disease. In some embodiment, the inflammatory condition includes a cytokine storm, or hyperinflammation, associate with the infectious disease. In some embodiments, the inflammatory condition includes acute lung injury or acute respiratory distress syndrome (ARDS).

In some embodiments, the infectious disease is an infection by, without limitation, one or more of the following pathogens: viruses (including but not limited to coronavirus, human immunodeficiency virus, herpes simplex virus, papilloma virus, parainfluenza virus, influenza virus, hepatitis virus, Coxsackie Virus, herpes zoster virus, measles virus, mumps virus, rubella, rabies virus, hemorrhagic viral fevers, H1N1, and the like), prions, parasites, fungi, mold, yeast and bacteria (both gram-positive and gram-negative). In some embodiments, pathogens include, without limitation, Candida albicans, Aspergillus niger, Escherichia coli (E. coli), Pseudomonas aeruginosa (P. aeruginosa), and Staphylococcus aureus (S. aureus), Group A streptococci, S. pneumoniae, Mycobacterium tuberculosis, Campylobacter jejuni, Salmonella, Shigella, and a variety of drug resistant bacteria.

In some embodiments, the inflammation is subsequent to or concurrent with an infection by a virus, e.g., a DNA or RNA virus. In some embodiments, the virus is an RNA virus, e.g., a single or double-stranded virus. In some embodiments, the RNA virus is a positive sense, single-stranded RNA virus. In some embodiments, the virus belongs to the Nidovirales order. In some embodiments, the virus belongs to the Coronaviridae family. In some embodiments, the virus belongs to the alphacoronavirus, betacoronavirus, gammacoronavirus or deltacoronavirus genus. In some embodiments, the alphacoronavirus is, without limitation, human coronavirus 229E, human coronavirus NL63 or transmissible gastroenteritis virus (TGEV). In some embodiments, the betacoronavirus is, without limitation, Severe Acute Respiratory Syndrome Coronavirus (SARS-CoV), SARS-CoV-2 (COVID-19), Middle Eastern Respiratory Syndrome Coronavirus (MERS-CoV), human coronavirus HKU1, or human coronavirus OC43. In some embodiments, the gammacoronavirus is infectious bronchitis virus (IBV).

The nucleic acid can be administered to the subject at any suitable amount. In some embodiments, the therapeutically effective amount of the nucleic acid includes about 0.01 μg, 0.02 μg, 0.05 μg, 0.1 μg, 0.2 μg, 0.5 μg, 1 μg, 2 μg, 3 μg, 4 μg, 5 μg, 6 μg, 7 μg, 8 μg, 9 μg, 10 μg, 15 μg, 20 μg, 25 μg, 30 μg, 40 μg, 50 μg, 75 μg. 100 μg, 125 μg, 150 μg, 175 μg, 200 μg, 250 μg, 300 μg, 400 μg, 500 μg, 600 μg, 700 μg, 800 μg, 900 μg, 1 mg, 2 mg, 3 mg, 4 mg, 5 mg, 10 mg, 15 mg, 20 mg, 30 mg, 40 mg, 50 mg, 75 mg, 100 mg or more, or an amount in a range defined by any two of the preceding values (e.g., 0.01 μg-0.1 μg, 0.1 μg-1 μg, 1 μg-10 μg, 10 μg-100 μg, 100 μg-1 mg, 1 mg-10 mg, 10 mg-100 mg). In some embodiments, the therapeutically effective amount of the nucleic acid includes about 0.001 μg/g, 0.002 μg/g, 0.005 μg/g, 0.01 μg/g, 0.02 μg/g, 0.05 μg/g, 0.1 μg/g, 0.15 μg/g, 0.2 μg/g, 0.5 g/g, 1 μg/g, 2 μg/g, 3 μg/g, 4 μg/g, 5 g/g, 6 μg/g, 7 μg/g, 8 μg/g, 9 μg/g, 10 μg/g, 15 μg/g, 20 μg/g, 25 μg/g, 30 μg/g, 35 μg/g, 40 μg/g, 45 μg/g, 50 μg/g, 60 μg/g, 70 μg/g, 80 μg/g, 90 μg/g, 100 μg/g of body weight, or more, or an amount in a range defined by any two of the preceding values (e.g., 0.001 μg/g-0.01 μg/g, 0.01 μg/g-0.1 μg/g, 0.1 μg/g-1 μg/g, 1 μg/g-10 g/g, 10 μg/g-100 μg/g). In some embodiments, the therapeutically effective amount of the nucleic acid is about 0.001 μg/g, 0.002 μg/g, 0.005 μg/g, 0.01 μg/g, 0.02 μg/g, 0.05 μg/g, 0.1 μg/g, 0.2 g/g, 0.5 g/g, or about 1 g/g of body weight, or more, or an amount in a range defined by any two of the preceding values (e.g., 0.001 μg/g-0.01 μg/g, 0.01 μg/g-0.05 μg/g, 0.05 μg/g-0.1 μg/g, 0.1 μg/g-0.2 μg/g, 0.2 μg/g-0.5 μg/g, or 0.5 μg/g-1 μg/g).

The nucleic acid or composition can be administered to the subject at any suitable dosing schedule. In some embodiments, the therapeutically effective amount of the nucleic acid or the composition is administered to the subject no more frequently than twice a week, once a week, once every two weeks, once every month, once every two months, once every three months, once every four months or longer, or at a frequency in a range defined by any two of the preceding values. In some embodiments, the nucleic acid is administered to the subject 1, 2, 3, 4, 5, 6, 7, 8, 9, 10, 15, 20, 25, 30 or more times. In some embodiments, the nucleic acid is administered to the subject at regular intervals.

The nucleic acid or composition can be administered using any suitable route. Administration can be local or systemic. In some embodiments, administration is parenteral. Suitable option for administration include, without limitation, intravenous, intramuscular, subcutaneous, intra-arterial, intraperitoneal, or oral administration. In some embodiments, the nucleic acid or composition is administered intravenously. In some embodiments, the nucleic acid or composition is administered by infusion. In some embodiments, the nucleic acid or composition is administered orally. Any suitable option for administering the nucleic acid or composition can be used. Non-limiting options for oral administration are provided in International Publication No. WO 2023/278802, filed Jun. 30, 2022, which is incorporated herein by reference.

Also provided herein is a method of promoting anti-inflammatory activity of regulatory T cells (Tregs) (also referred to herein Treg-modulating methods). The method generally includes contacting the nucleic acid or the composition of the present disclosure with a population of Tregs. In some embodiments, the nucleic acid induces changes in gene expression and/or epigenetic changes in Tregs that are exposed to the nucleic acids. In some embodiments, contacting the nucleic acid (or composition) increases expression of one or more of IL-10, ATG-7, and CCR6. In some embodiments, contacting the nucleic acid (or composition) increases transcription or translation of one or more of IL-10, ATG-7, and CCR6. In some embodiments, contacting the nucleic acid (or composition) increases transcription of one or more of IL-10, ATG-7, and CCR6 each independently by at least about 1.5 fold, 2 fold, 4 fold, 6 fold, 8 fold, 10 fold, 15 fold, 20 fold, 30 fold, 50 fold, 100 fold, 200 fold, 300 fold, 400 fold 500 fold, 1,000 fold, or more, or by a fold amount in a range defined by any two of the preceding values, compared to a suitable control, e.g., a Treg that has not been contacted with the nucleic acid.

In some embodiments, contacting the nucleic acid (or composition) increases secretion of interleukin 10 (IL-10) in the Tregs. In some embodiments, contacting the nucleic acid (or composition) increases secretion of IL-10 from the Tregs by at least about 1.2 fold, 1.5 fold, 2 fold, 2.5 fold, 3 fold, 4 fold, 5 fold, 6 fold, 8 fold, 10 fold, 15 fold, 20 fold, 50 fold, or more, or by a fold amount in a range defined by any two of the preceding values, compared to a suitable control, e.g., a Treg that has not been contacted with the nucleic acid.

The population of Tregs can be contacted with the nucleic acid or composition for any suitable amount of time. In some embodiments, the contacting is for 24 hours or more, 36 hours or more, 48 hours or more, 60 hours or more, 72 hours or more, or an amount of time in a range between any two of the preceding values.

In some embodiments, the contacting is done in vitro. In some embodiments, the contacting is done in vivo. In some embodiments, the contacting includes administering to a subject in need of treating an inflammation an effective amount of the nucleic acid or the composition. In some embodiments, the Treg is a human Treg. In some embodiments, the subject is a human subject. In some embodiments, the subject is a non-human subject, e.g., a non-human mammal. In some embodiments, the contacting is done ex vivo. In some embodiments, the Treg is autologous to the subject. In some embodiments, the method includes obtaining Tregs from the subject before contacting. In some embodiments, expanding Tregs obtained from the subject before contacting. In some embodiments, the Treg is heterologous to the subject. In some embodiments, the method includes administering Tregs that have been contacted with the nucleic acid or composition of the present disclosure to a subject in need thereof (e.g., a subject in need of treating an immune-related condition, as described herein).

Any suitable amount of nucleic acid can be contacted with the population of Tregs to promote the anti-inflammatory activity of the Tregs. In some embodiments, the effective amount depends on whether the contacting is done in vivo or in vitro. In some embodiments, the population of Tregs is contacted with, with about, or with at least 0.1, 0.2, 0.5, 1, 1.5, 2, 5, 10, 15, 20, 25, 30, 40, 50, 60, 70, 80, 90, or 100 nM, or with, with about, or with at most 1,000, 900, 800, 700, 600, 500, 400, 300, 200, or 100 nM of a nucleic acid of the present disclosure (e.g., BCYRN1, a nucleic acid containing a BDSS, a BDSS compound, etc.), or with a concentration in a range defined by any two of the preceding values (e.g., 0.1-1,000 nM, 1-500 nM, 1-100 nM, 10-200 nM, 50-800 nM, 50-200 nM, etc.). In some embodiments, the Treg cell is genetically modified to express a nucleic acid of the present disclosure (for example, via transfection with a vector configured to express a nucleic acid of the present disclosure (e.g., BCYRN1, a nucleic acid containing a BDSS, a BDSS compound, etc.)).

In any of the treatment methods, in some embodiments, the method includes administering a nucleic acid that acts as a microRNA sponge. Also provided is a method of treating an immune-related disorder comprising administering a therapeutically effective amount of an inhibitor of miR-138, miR-150 and/or miR-98 to a subject in need thereof. In some embodiments, the inhibitor of miR-138, miR-150 and/or miR-98 is a nucleic acid that binds to miR-138, miR-150 and/or miR-98, respectively. In any of the treatment methods, in some embodiments, the method includes administering BCYRN1, or a derivative thereof (e.g., a nucleic acid containing BDSS-138, BDSS-150, and/or BDSS-90, that is at most 35 nucleotides long). In any of the treatment methods, in some embodiments, the method includes administering a nucleic acid that specifically binds miR-138, miR-150 and/or miR-98. In any of the treatment methods, in some embodiments, the method includes administering a nucleic acid that specifically binds miR-138, miR-150 and/or miR-98. In some embodiments, miR-138, miR-150 and miR-98 are human miR-138, miR-150 and miR-98. In some embodiments, the nucleic acid comprises the miR-138 and miR-98 binding site ACAAC (SEQ ID NO: 18). In some embodiments, the nucleic acid comprises the miR-150 binding site GGGAG (SEQ ID NO: 19).

In some embodiments, alternative methods of sequence specific targeting of microRNAs are provided. In some embodiments, microRNA targeting includes the use of antisense RNA. In some embodiments, microRNA targeting includes the use of chemically modified antisense oligonucleotides. In some embodiments, microRNA targeting includes the use of locked nucleic acids (LNAs). In some embodiments, microRNA targeting includes the use of aptamers. In some embodiments, microRNA targeting includes the use of small interfering RNA. In some embodiments, microRNA targeting is performed in vitro. In some embodiments, microRNA targeting is performed in vivo.

In some embodiments, BCYRN1, or a derivative thereof, e.g., a synthetic derivative thereof, comprises a miRNA-138, miR-150 and/or miR-98-binding nucleic acid. In some embodiments, a miRNA-138, miR-150 and/or miR-98-binding nucleic acid binds to miRNA-138, miR-150 and/or miR-98 with a binding affinity of 10⁻⁵ M-10⁻¹²M, e.g., 10⁻⁶M to 10⁻¹¹M. In some embodiments, the nucleic acid that specifically binds miRNA-138, miR-150 and/or miR-98 inhibits or reduces function and/or expression of miRNA-138, miR-150 and/or miR-9. In some embodiments, the nucleic acid that specifically binds miRNA-138, miR-150 and/or miR-98 is an RNA. In some embodiments, the nucleic acid that specifically binds miR-138, miR-150 and/or miR-98 is a non-coding RNA. In some embodiments, the nucleic acid that specifically binds miR-138, miR-150 and/or miR-98 is any one of the nucleic acids of the present disclosure derived from BCYRN1 (e.g., BDSS-138, BDSS-150, and/or BDSS-150).

In some embodiments, BCYRN1, or a derivative thereof (e.g., BDSS-138, BDSS-150, and/or BDSS-150), serves as a microRNA sponge. In some embodiments, BCYRN1, or a derivative thereof, augments Treg cell proliferation, migration and/or IL-10 production. In some embodiments, administration of BVYRN1, or a derivative thereof, augments regulatory T cell function (proliferation, infiltration, and activation) acutely. In some embodiments, BCYRN1 or a derivative thereof, regulates proliferation by competitively binding with miR-138 to regulate ATG-7 expression and ATG-7 induced autophagy. In some embodiments, BCYRN1, or its derivatives, regulates Treg migration by competitively binding with miR-150 to regulate CCR6 expression and CCR6-dependent Treg migration. In some embodiments, BCYRN1, or its derivatives, mediates induction of IL-10 in Treg by competitively binding with miR-98 to regulate IL-10 expression. In some embodiments, BCYRN1, or its derivatives, is itself useful therapeutically. In some embodiments, levels of BCYRN1, or its derivatives, is enhanced in EVs to accentuate their efficacy in a therapeutic context.

In some embodiments, the formulations provided for herein allow the use of nucleic acids in treating immune-related disorders where inflammation and/or tissue injury are the main drivers of pathology. In some embodiments, conditions treated using such formulations, include, without limitation, inflammatory disease, cardiac injury, autoimmune disease, or transplant rejection. In some embodiments, the formulations have cardioprotective effects when administered to a subject suffering from cardiac injury due to, without limitation, myocardial infarction and/or heart failure, among other maladies.

### Kits

Also provided herein are kits that include one or more nucleic acids (e.g., BCYRN1, or nucleic acid having a BDSS as described herein) compositions, or vectors of the present disclosure. The present kit in some embodiments finds use in treating an immune-related disorder or an inflammatory condition, as provided herein. A kit can include one or more nucleic acids of the present disclosure (e.g., BCYRN1, or nucleic acid having a BDSS as described herein), compositions, or vectors of the present disclosure, and a transfection reagent. The transfections reagent can be any suitable transfection reagent, as provided herein. In some embodiments, the transfection reagent includes one or more of a lipid (e.g., a liposome-forming lipid), liposome, lipid nanoparticle (LNP), a PEGylated lipid, and an extracellular vesicle. In some embodiments, the nucleic acid is in solution. In some embodiments, the nucleic acid is in lyophilized form. In some embodiments, the kit includes a pharmaceutically acceptable excipient, as provided herein. Kits can include one or more containers (e.g., vials, ampoules, test tubes, flasks or bottles) for holding one or more components of the kits. The kits may further include instructions for using the kit to treat a condition (e.g., myocarditis). The information and instructions may be in the form of words, pictures, or both, and the like.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

### EXAMPLES

### Materials and Methods:

The following materials and methods were used in the Examples.

Animals: Male 10-12-week-old FOXP3-IRES-mRFP mice, purchased from Jackson Laboratory, were used for in vivo experiments. All animal procedures were performed according to protocols approved by the Institutional Animal Care and Use Committee (IACUC) at Cedars-Sinai Medical Center (IACUC: 009183).

Myocardial infarction mouse model: MI was induced in 10-12-week-old male mice as reported (Cambier et al., 2017). Briefly, a left parasternal thoracotomy was performed under anesthesia. A ligature was tightened around the left anterior descending coronary artery (LAD) for 45 min of ischemia, then released to allow reperfusion. Fifteen minutes later, 100 μl of each test item (EV-BCYRN1 or CDC-EVs, 2*10\hat{ }9 EVs/mouse) or vehicle (inIscove's modified Dulbecco's media, IMDM) was administered by intravenous (IV) injection.

Cell Culture: CDCs were isolated as described (Makkar et al, 2012). Briefly, human heart tissue, obtained under an IRB-approved protocol, was chopped into fragments, and enzymatically digested with collagenase. Tissue was then cultured as explants on fibronectin-coated flasks (Corning, cat #356009) 37° C., 5% C02, 5% 02 in IMDM with 20% fetal bovine serum (FBS) and penicillin (100 U/mL). After 2-3 weeks, an outgrowth of stromal-like cells and phase-bright round cells from the tissue fragments reached 80% confluence. These cells were then harvested and seeded onto Ultra-Low Attachment flasks to support cardiosphere formation. Two days later, CDCs were formed by seeding cardiospheres on fibronectin-coated flasks and cultured IMDM with 20% FBS and expanded to passage 4-6 for EV isolation.

Human induced Treg cells were purchased from IQ Biosciences (Cat #IQB-Hu1-iTr-1, Berkeley, CA, USA) and expanded (Treg Expansion Kit, Miltenyi Biotec, Cat #130-095-353, Santa Barbara, CA) according to the manufacturer's protocol. Human Treg cells were used within 5 passages.

Extracellular vesicle (EV) isolation: EVs were prepared from FBS-depleted media conditioned by CDCs using ultrafiltration by centrifugation as described (Akhmerov et al., 2021). Briefly, 15 days' conditioned media was harvested, centrifuged at 1,000×g for 10 min to eliminate cells, followed by filtration through a 0.22-μm filter to remove cell debris. Then the CDC-EVs were concentrated using 100 Kda filters (Millipore) at 3000×g for 30 min. EVs were assessed using a nanoparticle tracking system.

Overexpression of BCYRN1 in human induced Treg cells: For lentiviral vector overexpressing BCYRN1 (Applied Biological Materials Inc. [abm]), the full-length BCYRN1 cDNA was inserted into the pLenti-CMV-GFP-2A-Puro vector (abm) at EcoRV sites, and BCYRN1 expression was driven by a CMV promoter. In this study, human induced Treg cells were transfected with lentiviral BCYRN1 vector using DharmaFECT Transfection Reagent (PerkinElmer). An empty vector (pLenti-CMV-GFP-2A-Puro) served as a negative control.

Cell proliferation assay: Cell proliferation was assessed using Cell Counting Kit-8 (Sigma-Aldrich) according to the manufacturer's instructions (Liao et al, 2020). Briefly, human induced Treg cells were seeded in 96-well plates at a density of 5000 cells/well, followed by addition of CDC-EVs (1000 particles/cell), transfecting lentiviral BCYRN1 vector or empty lentiviral vector for 72 hours. Then 10 μL CCK-8 solution (water-soluble tetrazolium salt, WST-8) was added into each well for 4 hours incubation, WST-8 was reduced by dehydrogenase activities in living cells to give a yellow-color formazan dye. Afterwards, spectrophotometric absorbance was measured at 450 nm for each well. All the experiments were repeated three times, each sample in triplicate. Mean±S.D. is presented for pooled data.

Transwell migration: Boyden chambers (Corning Costar) were used to determine the transmigration of Treg cells (Cook et al, 2014). Briefly, human induced Tregs were seeded (5×10\hat{ }5 cells/well) onto 6.5 mm transwell inserts with PET membrane (Cat #3464, Costar, Corning, USA). Culture medium (600 μL) with 500 ng/mL of recombinant mouse CCL20 was placed in the lower chamber. The transwell plates were incubated for 5 h at 37° C., followed by quantification of migrated Treg cells in the lower chamber. Data represent results obtained from three biological replicates, and each biological replicate comprised 2-3 technical replicates.

Luciferase activity assays: A 40 bp/44 bp LncRNA BCYRN1 3′UTR segment containing the putative miR-150 target site (sense 5′-AAACTAGCGGCCGCTAGTGAGGCTAAGAGGCGGGAGGATT-3′ (SEQ ID NO:20) and antisense 5′-CTAGAATCCTCCCGCCTCTTAGCCTCACTAGCGGCCGCTAGTTT-3′ (SEQ ID NO:21)), miR-138 target site (5′-AAACTAGCGGCCGCTAGTTCCCTCAAAGCAACAACCCCCT-3′ (SEQ ID NO:22) and antisense 5′-CTAGAGGGGGTTGTTGCTTTGAGGGAACTAGCGGCCGCTAGTTT-3′ (SEQ ID NO:23)), miR-98 target site (sense 5′-AAACTAGCGGCCGCTAGTACTTCCCTCAAAGCAACAACCT-3′ (SEQ ID NO: 40) and antisense 5-CTAGAGGTTGTTGCTTTGAGGGAAGTACTAGCGGCCGCTAGTTT (SEQ ID NO:25)) was cloned into the PmeI and XbaI sites of the pmirGLO vector (Cat. #E1330, Promega). For pmirGLO-BCYRN1 3′ UTR-miR-138-target-mutant segment (sense 5′-AAACTAGCGGCCGCTAGTTCCCTCAAAGCATGTTGCCCCT-3′ (SEQ ID NO:26) and antisense 5′-CTAGAGGGGCAACATGCTTTGAGGGAACTAGCGGCCGCTAGTTT-3′ (SEQ ID NO:27)), the miR-138 target site (ACAAC) within the BCYRN1 3′UTR was changed to (TGTTG) (SEQ ID NO:32), for pmirGLO-BCYRN1 3′ UTR-miR-150-target-mutant segment (sense 5′-AAACTAGCGGCCGCTAGTGAGGCTTTCACGCCCCTCGATT-3′ (SEQ ID NO:28) and antisense 5′-CTAGAATCGAGGGGCGTGAAAGCCTCACTAGCGGCCGCTAGTTT-3′ (SEQ ID NO:29), the miR-150 target site (GGGAG) within the BCYRN1 3′UTR was changed to (CCCTC) (SEQ ID NO:33), and for pmirGLO-BCYRN1 3′UTR-miR-98-target-mutant segment (sense 5′-AAACTAGCGGCCGCTAGTACTTCCCTCAAAGCATGTTGCT-3′ (SEQ ID NO:30) and antisense 5′-CTAGAGCAACATGCTTTGAGGGAAGTACTAGCGGCCGCTAGTTT-3′ (SEQ ID NO:31), the miR-98 target site (ACAAC) within the BCYRN1 3′UTR was changed to (TGTTG) (SEQ ID NO:32). Followed the manufacturer's protocol (Promega), Treg cells were seeded into 24-well plates. Two days after co-transfecting Treg cells in 24-well plates with either miR-138, miR-98, and miR-150 mimic or a mimic RNA negative control, along with either a pmirGLO-BCYRN1-3′ UTR-miRNA-target or pmirGLO-PTEN-3′UTR-BCYRN1-miRNA target-mutant luciferase reporter vector using DharmaFECT 1 Transfection Reagent (PerkinElmer), luciferase activity was measured using the Dual-Luciferase Reporter Assay (Cat. #E2940, Promega). Firefly luciferase activity was normalized by Renilla luciferase activity and expressed as a percentage of control (Triplicated independent experiment, performed in 3 wells each time).

Flow cytometry: The following antibodies were used for staining: Brilliant Violet 421™ anti-mouse CD4 Antibody (clone GK1.5; BioLegend), PE anti-mouse CD25 Antibody (PC61; BioLegend), Alexa Fluor®594-conjugated anti-Mouse FoxP3 Antibody (clone 1054C; R&D Systems), APC/Cyanine7 anti-mouse CD45 Antibody (clone 30-F11; BioLegend), Alexa Fluor 700-conjugated anti-IL-10 (clone JES5-16E3; eBioscience). Viability staining was performed with Zombie Aqua (BioLegend), and proliferation assays were performed (In Vivo EdU Flow Cytometry Kit 647, Sigma-Aldrich). According to manufacturers' protocols, transcription factor staining buffer set (eBioscience) or intracellular fixation and permeabilization buffers (BioLegend) were used for intracellular staining of IL-10 and FOXP3. Samples were acquired using the Sony SA3800 spectral analyzer and analyzed with FlowJo Software.

2,3,5-Triphenyl-2H-tetrazolium chloride staining: MI was induced as described earlier. Three days post-MI, hearts were arrested in diastole (10% KCl), excised, PBS washed, and cut into serial sections of ≈1l mm thickness. The sections were immersed in 2,3,5-Triphenyl-2H-tetrazolium chloride (TTC, 1% solution in PBS) for 15-20 minutes in the dark at 37° C. Surviving tissue turned a deep red, while necrotic tissue remained pale. Once the color was established the sections were fixed in 4% formalin for ˜20 minutes followed by PBS wash. Then the sections were imaged and weighed.

ELISA assays: Concentration of cTnI in serum was measured by a mouse-specific ELISA (Cat #CTNI-1-HSP, Life Diagnostics) according to the manufacturer's instructions. Culture supernatants were collected from human Treg cells exposed to various treatments and IL-10 was quantified using the human IL-10 Elisa kit (cat #KHC0101C, Thermo Scientific) according to the manufacturer's instructions.

Western blotting: At the end of each protocol, human Treg cells were lysed using RIPA buffer (Cat #: PI89900, Fisher Scientific), as described (Liao et al., 2016). Equal amounts of the proteins were electrophoresed in an SDS-polyacrylamide gel under reducing conditions followed by transfer to PVDF membranes. Blots were blocked with 3% BSA in TBS-Tween 20, and probed with antibodies specific for ATG7 (1:1000; cat #: MAB6608; Novus Biologicals), LC3b (1:1,000; Cat #: NB 100-2220; Novus Biologicals), P62 (1:1,000, Cat #: PM045; MBL International), and β-actin (1:5,000; Cat #: MA5-15739-HRP; Thermo Scientific). Secondary antibodies were alkaline phosphatase conjugated to goat anti-mouse/rabbit IgG (1:10,000; Jackson ImmunoResearch Labs). Membranes were washed and incubated with an ECL solution and signals were detected using Supersignal West Dura Extended Duration or Pico PLUS Chemiluminescent Substrate (Thermo Fisher Scientific, Waltham, MA) and imaged on a Bio-Rad ChemiDoc imaging system. All experiments had at least three biological replicates; representative blots are shown in the figures.

RNA preparation and next Generation sequencing and analysis: EV RNAs and Treg cell RNAs were extracted in QIAzol (QIAGEN) and isolated using the Rneasy Mini Kit (QIAGEN) per manufacturer's instructions. The total RNA yields were quantitated by NanoDrop A260 for overall recoveries. Total RNA was assessed for quality, enriched, fragmented, ligated with adapters, and converted to cDNA. The cDNA was barcoded, amplified, and the RNA-seq libraries were assessed. Raw sequencing data were multiplexed and processed into FASTQ format using bcl2fastq v2.20 (Illumina, San Diego, California). Reads of EV RNA-seq were then aligned to a comprehensive non-coding RNA (ncRNA) database and annotated (Cambier et al., 2017). Reads of Treg cell RNA-seq were aligned to the mouse GRCm38 transcriptome using STAR/RSEM. Gene expression counts were normalized using a modified trimmed mean of the M-values normalization method. The Wald test was used to assess the differential expressions between two sample groups by DESeq2 (Akhmerov et al., 2021). Genes with an absolute log 2 fold change (|log 2 fold change|) higher than 2.5 and P-value <0.05 compared to the control samples were defined as differentially expressed and were analyzed by the Ingenuity Pathway Analysis (IPA) software (version 2021; Ingenuity Systems; QIAGEN). Differentially expressed Genes (DEGs) from RNA sequencing outcomes were uploaded to IPA for disease functions analysis and heatmap generation.

qPCR: For quantitative analysis of mRNA and miRNA expression, comparative real-time PCR was performed with the use of Taqman Universal PCR Master Mix (Applied Biosystems). Specific primers and probes for IL-10, CCR5, CCR6, CCR7, LncRNA BCYRN1, GAPDH, mature miR-138, -150, -98 and snRNA RNU6B (U6) were obtained from Applied Biosystems. All reactions were run in triplicate. The amount of miRNA was obtained by normalizing to snRNA RNU6B and relative to control as reported (Hu et al, 2017).

### Example 1

This nonlimiting example shows CDC-EVs induced Treg proliferation, migration, and upregulation of IL-10.

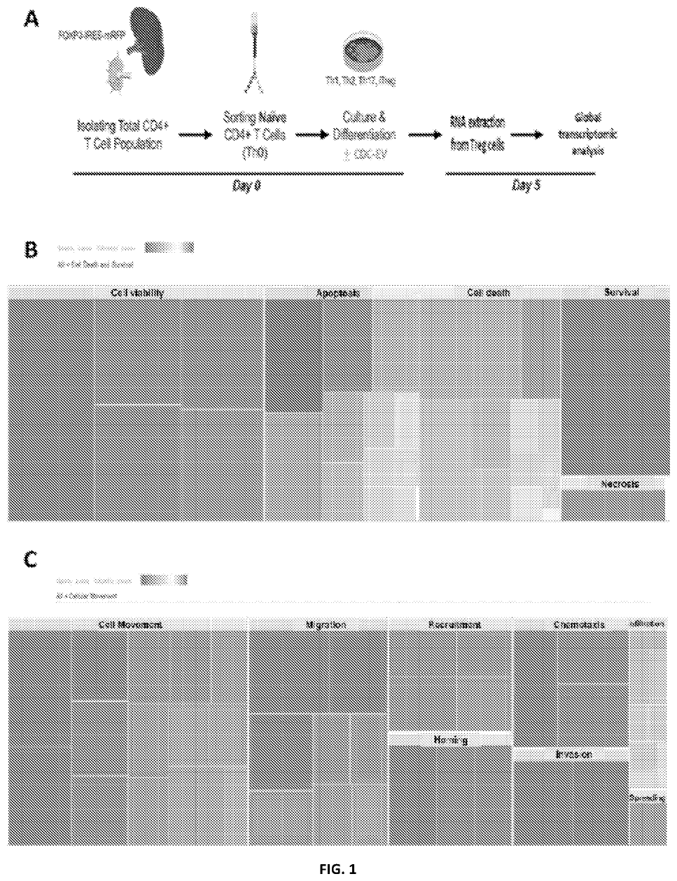

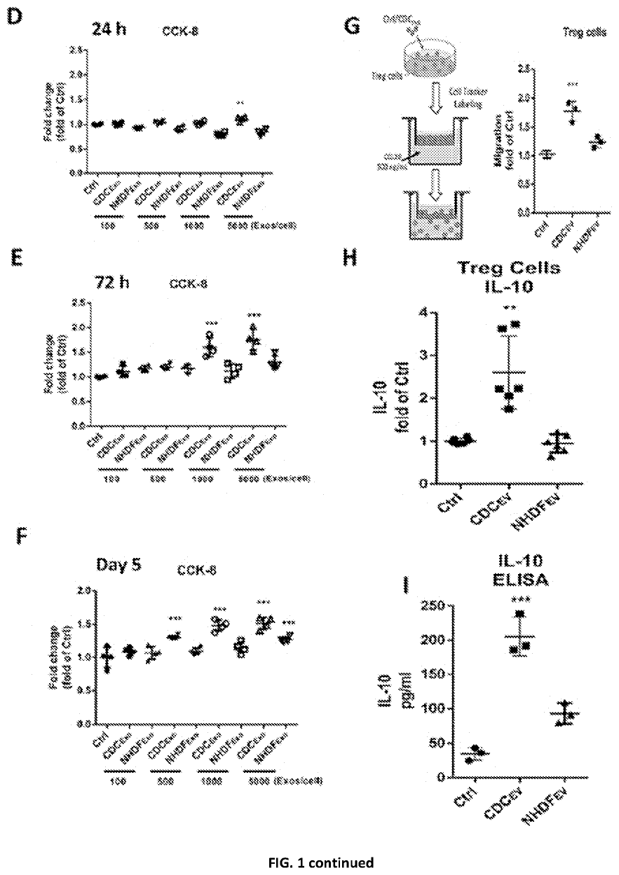

In a previous experiment, naïve mouse CD4+ T cells were isolated from mice and differentiated into conventional effector T cells (Th1, Th2, Th17) or induced regulatory T (iTreg) cells (FIG. 1 panel A). After 5 days of culture, exposure of cells to CDC-EVs resulted in distinct, dose-dependent responses among the different types of CD4+ T cells: no change in Th1 and Th2 proliferation; decreased proliferation of Th17 cells; and increased proliferation of iTreg cells (dosing at 1000 EVs/cell elicited a strong proliferative response). By RNA sequencing (RNAseq), it was discovered that CDC-EVs could augment IL-10 production by Tregs. New ingenuity pathway analysis (IPA) on the RNAseq data (FIG. 1 panels B and C) from iTreg cells revealed several significantly upregulated categories (based on Z-score), which are represented by progressively gray colors as the upregulation increases. Conversely, decreased expression is depicted in light gray. Heat maps showed that CDC-EVs increased the expression of genes promoting cell survival and viability, but decreased genes related to apoptosis and necrosis (FIG. 1 panel B). Additionally, CDC-EVs strongly induced the expression of genes promoting cell movement and migration (FIG. 1 panel C). New experiments were performed to test whether CDC-EVs induce cell proliferation, migration, and IL-10 production in human iTregs exposed to different doses (0-5000 EVs/cell) of CDC-EVs for various durations (0-5 days). Normal human dermal fibroblast Evs (NHDf-EVs) served as a negative control; these EVs are similar in size distribution but functionally inert in the relevant assays (Ibrahim et al, 2014). Exposure of human iTregs to CDC-EVs resulted in a dose-dependent increase in cell number over time (FIG. 1 panels D-F). Transwell experiments confirmed that CDC-EVs induced human iTreg migration (FIG. 1 panel G), IL-10 gene expression (FIG. 1 panel H), and IL-10 protein secretion (FIG. 1 panel I).

### Example 2

This nonlimiting example shows CDC-EV-mediated expression of BCYRN1 in human iTreg cells.

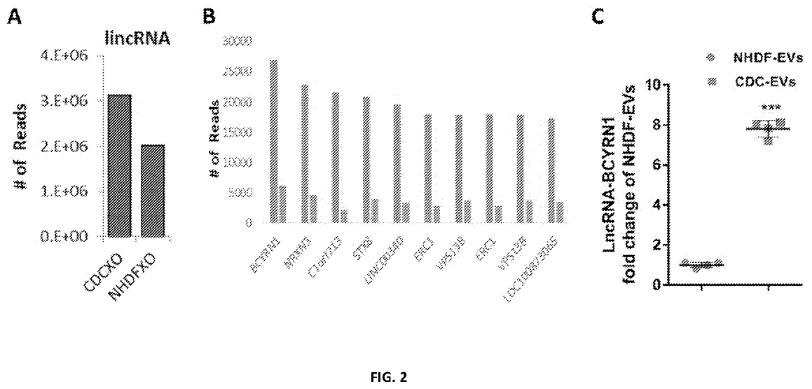

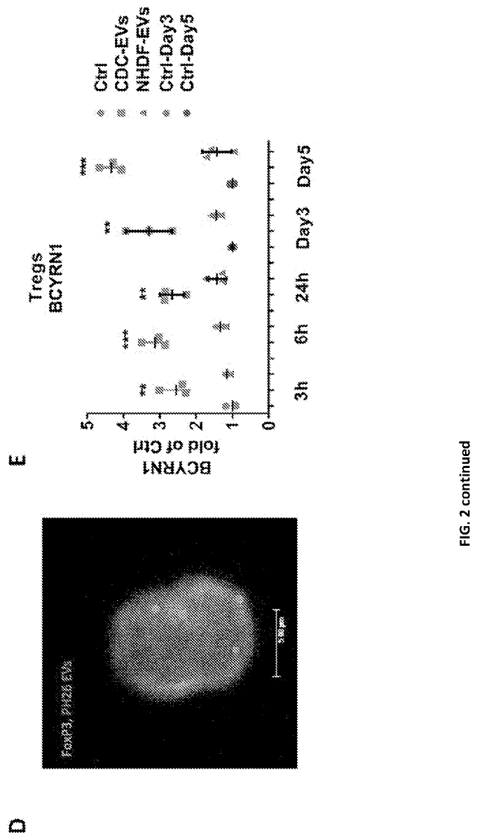

To dissect the mechanism whereby CDC-EVs enhance human iTregs, RNAseq was performed. Evs contain many (typically >10,000) molecularly distinct ncRNA entities with known or plausible bioactivity. RNA seq revealed that lncRNAs are abundant in CDC-EVs (˜50% more plentiful than in inert NHDf-Evs; FIG. 2 panel A). RNAseq library data (FIG. 2 panel B) quantifying lncRNAs from CDC-EVs (left bars) and NHDF-EVs (right bars) revealed one particular lncRNA species BCYRN1 to be the most plentiful lncRNA in CDC-EVs, a finding confirmed by qPCR (FIG. 2 panel C). Next, it was investigated whether BCYRN1 within CDC-EVs could be transferred from CDCs to human iTregs, leading, in turn, to enhanced levels of BCYRN1 in recipient cells. Human iTregs (labeled for Treg marker-Foxp3 in green) exposed to CDC-EVs and labeled with PKH26 (Red) revealed cytoplasmic puncta consistent with EV internalization (FIG. 2 panel D). Exposure to unlabeled CDC-EVs resulted in time-dependent increases in BCYRN1 in human iTregs (FIG. 2 panel E). Cellular levels of BCYRN1 were enhanced up to 4-fold; the effect was selective, as exposure to NHDf-EVs caused no significant changes in BCYRN1 levels. Thus, BCYRN1 is highly enriched in CDC-EVs; uptake of these EVs increases BCYRN1 levels in human iTregs.

### Example 3

This nonlimiting example shows CDC-EV-mediated human iTreg proliferation, migration, and induction of IL-10 involve BCYRN1.

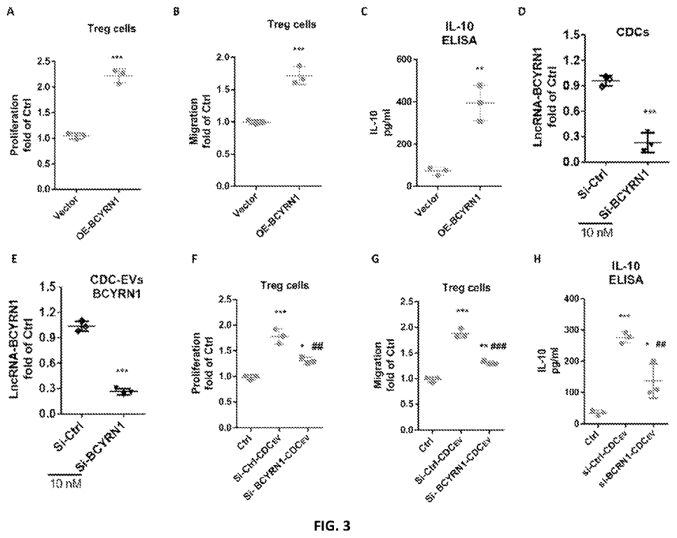

Next sought it was determined whether BCYRN1 was responsible for CDC-EV-induced upregulation of human iTregs. For this assessment, the functional implications of BCYRN1 were investigated by directly transfecting human iTregs with either empty lentiviral vector or vector expressing BCYRN1. Overexpression of BCYRN1 increased human iTreg proliferation (FIG. 3 panel A), migration (FIG. 3 panel B) and IL-10 production (FIG. 3 panel C). To further probe BCYRN1's role in human iTreg modulation, siRNA was used to suppress BCYRN1. Transfection of CDCs with siRNA against BCYRN1 (si-BCYRN1) for 48 hours, followed by EV isolation, revealed decreased expression of BCYRN1 both in CDCs (FIG. 3 panel D) and CDC-EVs (FIG. 3 panel E) relative to siRNA-control (si-ctrl) group. In terms of Treg proliferation, migration, or IL-10 production, EVs with siRNA-suppressed BCYRN1 elicited much weaker responses than CDC-EVs with normal BCYRN1 levels (i.e., those from CDCs exposed only to si-ctrl; FIG. 3 panel F-H). Thus, BCYRN1 underlies the ability of CDC-EVs to enhance human iTreg number and bioactivity.

### Example 4

This nonlimiting example shows CDC-EV BCYRN1 induced ATG7-dependent autophagy by sponging miR-138.

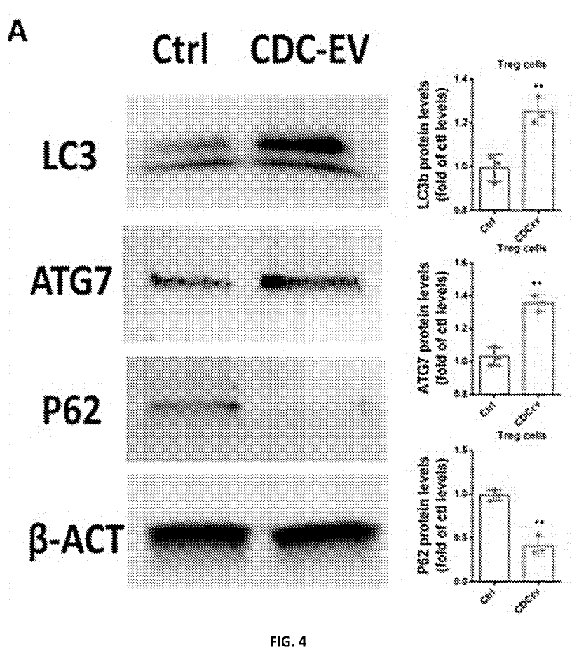

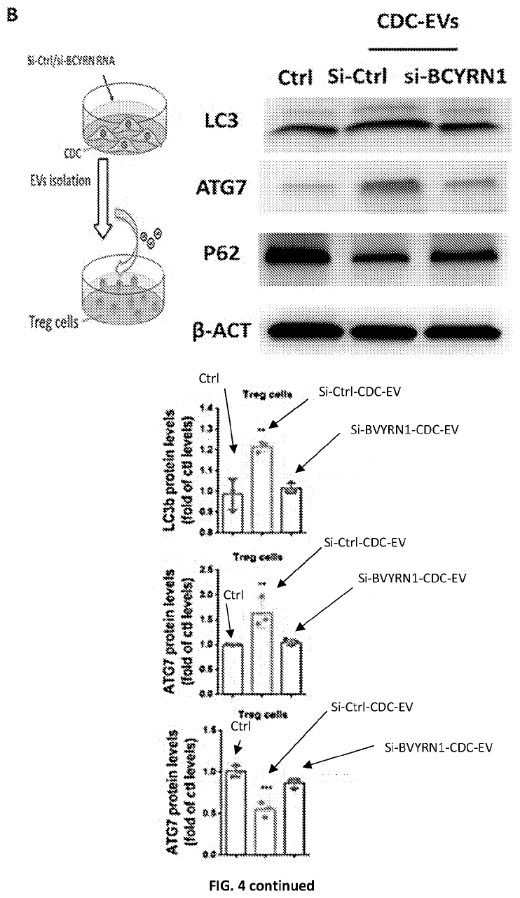

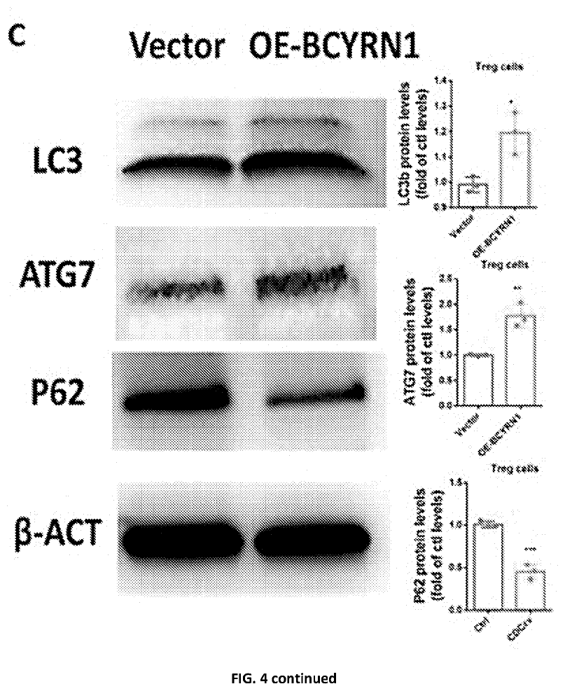

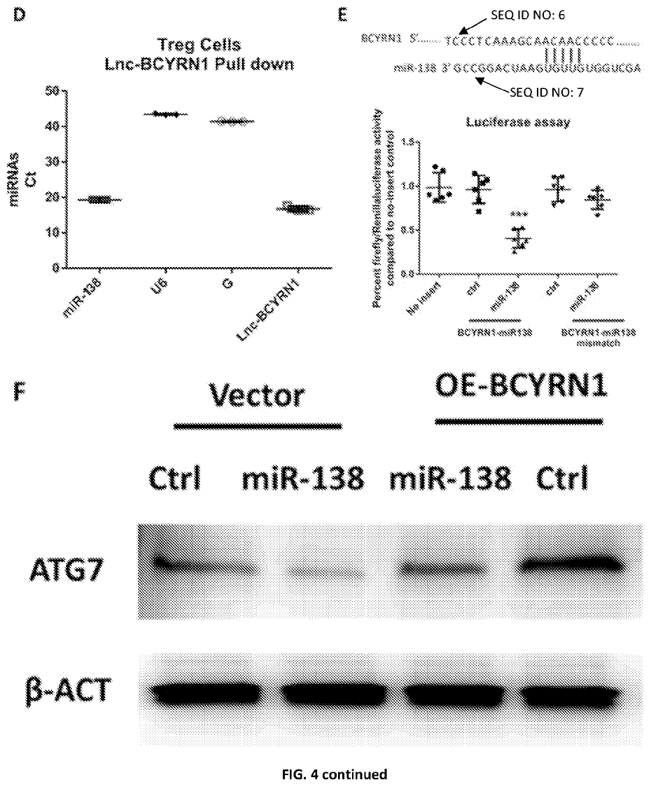

ATG7-dependent autophagy is essential to the survival and proliferation of Treg cells (Wang et al, 2021). To determine if CDC-EVs promote autophagy, protein levels of classic autophagic markers in human iTreg were measured. As FIG. 4 panel A shows, CDC-EVs increased ATG7 and LC3b, whereas P62, a marker of autophagic flux, was decreased, consistent with the notion that CDC-EVs induced autophagy. When applying EVs in which BCYRN1 had been suppressed by siRNA, autophagy induction was blunted (FIG. 4 panel B). Conversely, BCYRN1 overexpression increased autophagy markers in iTregs (FIG. 4 panel C). One way that lncRNAs, including BCYRN1, alter gene expression is by acting as “sponges” for specific miRNAs (Bossi & Figueroa-Bossi, 2016; Mu et al, 2020). Because BCYRN1 is predicted to bind miR-138 (Jeggari et al, 2012), RNA pull-down was used to test this prediction. After incubating biotin-labelled BCYRN1 probe with lysates of iTreg cells overexpressing BCYRN1, the precipitates were enriched in miRNA-138 (and BCYRN1, as a positive control), while negative controls GAPDH and U6 were undetectable (Ct values >40; FIG. 4 panel D).

Interactions between BCYRN1 and miR-138 were further assessed by mutating the potential miR-138 binding site within BCYRN1 and gauging reporter gene expression. Co-transfection of the wild type (WT) or mutant luciferase reporters with miR-138 into HEK-293 cells revealed that miR-138 reduced the activity of the WT reporter by over 50% but had no effect on the mutant reporter (FIG. 4 panel E). ATG7 is a known target of miR-138. Indeed, ATG7 was suppressed by miR-138, an effect reversed by overexpressing BCYRN1 (FIG. 4 panel F). Thus, the data support the concept that BCYRN1 increases human iTreg proliferation by sponging miR-138 to induce ATG7-dependent autophagy.

### Example 5

This nonlimiting example shows CDC-EV BCYRN1 induced CCR6-dependent migration by sponging miR-150.

CCR6 recruits Treg cells into inflammatory tissue (Lim et al, 2006; Yamazaki et al, 2008). Indeed, CCR6 transcript levels were elevated in human iTreg cells exposed to CDC-EVs (FIG. 5 panel A). To probe the role of BCYRN1, human iTreg cells exposed to either si-ctrl-CDC-EVs or si-BCYRN1-CDC-EVs were studied (BCYRN1 knock-down in CDC-EVs). As shown in FIG. 5 panel B, si-ctrl-CDC-EVs (i.e., “normal” EVs) upregulated CCR6, but this effect was much reduced after suppression of BCYRN1 in human iTregs exposed to si-BCYRN1-CDC-EVs. Direct transfection with a BCYRN1-expressing lentiviral vector likewise increased CCR6 transcript levels, but empty vector did not (FIG. 5 panel C). Analysis of lncRNA-miR interactions predicted miR-150 as a sponge target of BCYRN1 (Jeggari et al., 2012); in turn, CCR6 is a potential target of miR-150. RNA pull-down with a biotin-labeled BCYRN1 probe showed miR-150 and BCYRN1 (positive control) were highly enriched in the precipitates, while negative controls GAPDH and U6 were not detectable (FIG. 5 panel D). To further probe the interaction between BCYRN1 and miR-150, the binding site within BCYRN1 for miR-150 was mutated (Lang et al, 2020) and luciferase expression was measured. While miR-150 reduced the activity of the WT reporter, the mutant reporter was not affected. Consistent with an earlier report (Ito et al, 2014), it was found that miR-150 targets CCR6; overexpression of BCYRN1 attenuated miR-150's ability to reduce CCR6 (FIG. 5 panel F), while boosting migration of human iTregs (FIG. 5 panel G) Taken together, the data demonstrate that BCYRN1 could sponge miR-150 to induce CCR6-dependent migration of human iTreg.

### Example 6

This nonlimiting example shows CDC-EV BCYRN1 induced expression of IL10 by sponging miR-98.

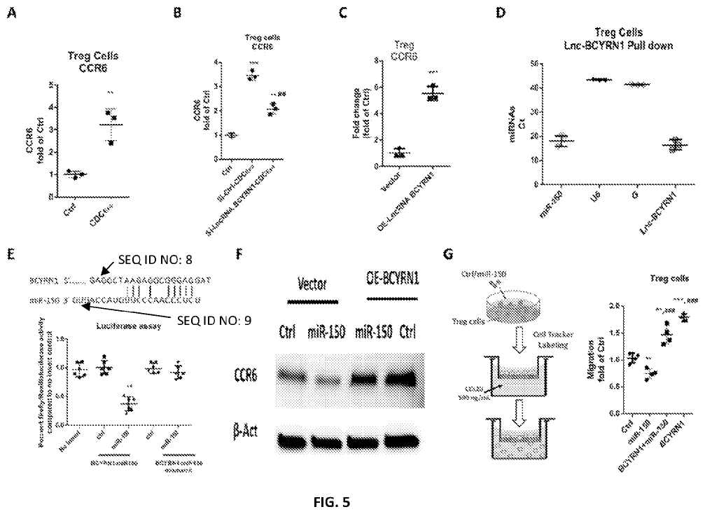

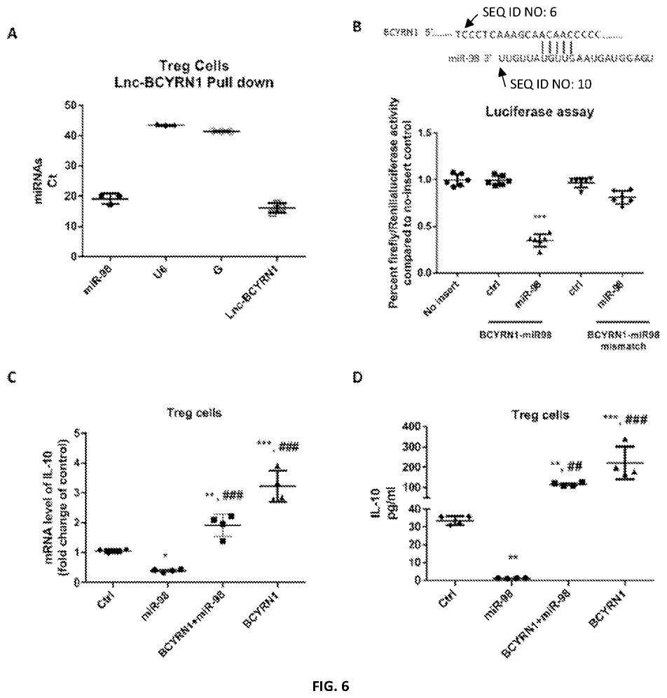

Analysis of lncRNA-miR interactions predicted BCYRN1 could sponge miR-98 (Jeggari et al., 2012), a miR which, in turn, targets IL-10 (Liu et al, 2011). RNA pull-down precipitates were enriched in miR-98 (and BCYRN1, as a positive control), while negative controls GAPDH and U6 were undetectable (Ct values >40; FIG. 6 panel A). Additionally, in a luciferase reporter system, miR-98 reduced the activity of the WT reporter, but not a mutant reporter in which the miR-98 binding site within BCYRN1 had been disrupted (FIG. 6 panel B). Furthermore, IL-10 was suppressed by miR-98, an effect reversed by overexpressing BCYRN1 (FIG. 6 panels C and D). Thus, BCYRN1 increases expression of IL-10 in human iTreg by sponging miR-98.

### Example 7

This nonlimiting example shows therapeutic efficacy of CDC-EVs and CDC-EV-BCYRN1 in MI.

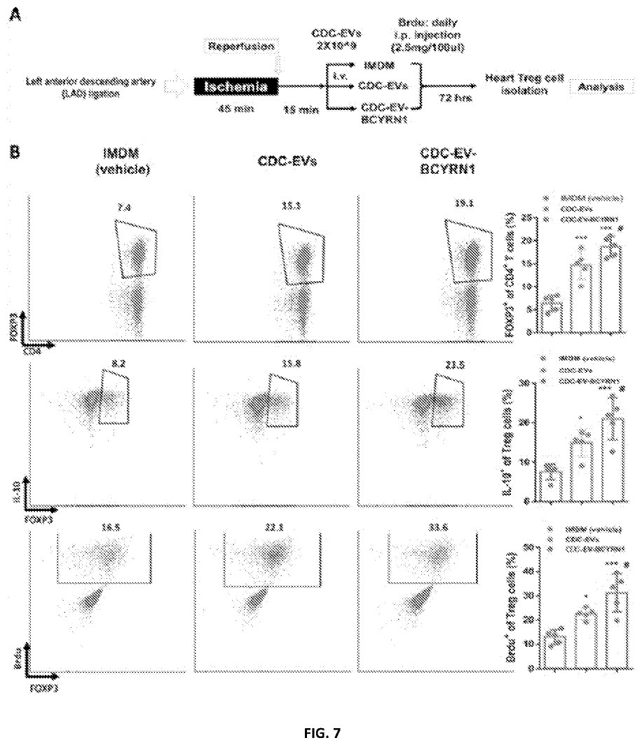

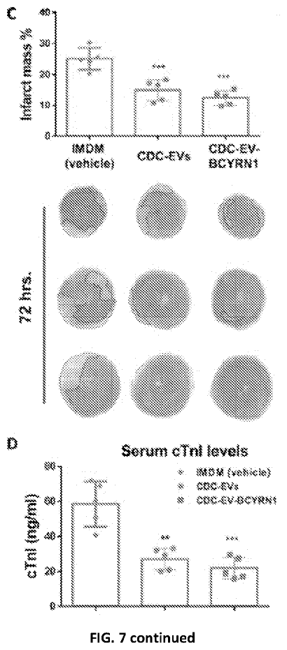

CDC-EVs reduce infarct size in rats post-MI (Cambier et al., 2017). Here BCYRN1 was demonstrated to be plentiful in CDC-EVs, and, on its own, BCYRN1 can upregulate human iTregs. Thus, the hypothesis that an increase in Tregs underlies the cardioprotective effects of CDC-EVs in MI was tested. Fifteen minutes post-MI, mice received I.V. CDC-EVs, CDC-EVs with BCYRN1 over-expression, or vehicle (IMDM; FIG. 7 panel A). Infarct size, circulating TnI I levels, and infiltrating Treg were assessed 72 hours later. The number of CD4+FOXP3+IL10+ cells was increased in mice given CDC-EVs, and even higher in mice that received CDC-EVs with BCYRN1 overexpression, relative to vehicle controls (FIG. 7 panel B). Thus, CDC-EVs and CDC-EVs overexpressing BCYRN1 promote Treg cell infiltration into the heart and augment IL-10-producing Treg cells. Additionally, CDC-EVs, and CDC-EVs with BCYRN1 overexpression, increased the number of CD4+FOXP3+Brdu+ cells (i.e., proliferating Tregs) in the heart. Either CDC-EVs or CDC-EVs with BCYRN1 overexpression significantly decreased infarct size and serum TnI levels compared to vehicle control (FIG. 7 panels C and D). The extent of scar size reduction was so large (>50% relative reduction) that it is perhaps not surprising that the cardioprotective effects of either intervention (viz., CDC-EVs with or without enhanced BCYRN1 expression) were comparable.

### Discussion Regarding Examples 1-7

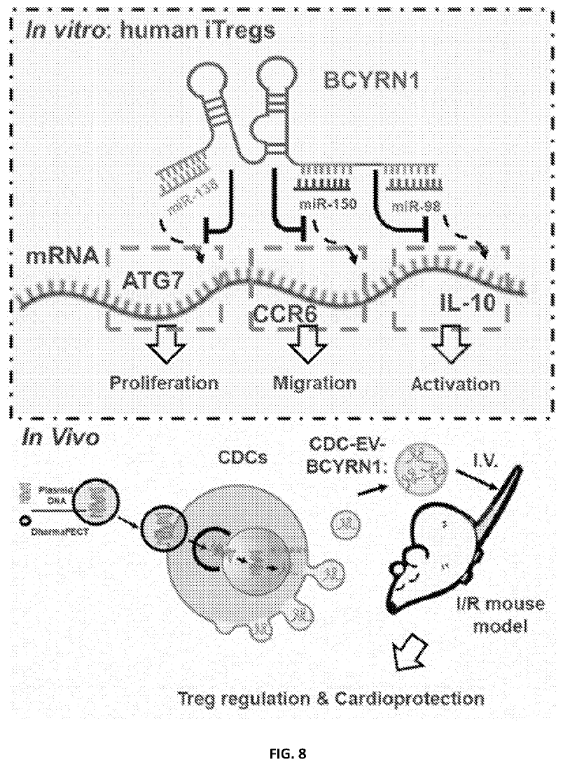

MI, known as “heart attack” in the vernacular, is a leading cause of death in the US (Tsao et al, 2022). Tregs have the potential to promote tissue repair and functional recovery post-MI (Li et al, 2018), but it can be difficult, costly and time-consuming to isolate and expand Treg cells ex vivo for clinical use (Raffin et al., 2020). A therapy that can selectively and quickly expand and activate Tregs in vivo is highly desirable. EVsHere, BCYRN1, a lncRNA plentiful in CDC-EVs, was pinpointed as an agent for expanding and activating Tregs. Acting as a miR sponge, BCYRN1 suppresses miR-138 (targeting ATG7-dependent autophagy), miR-150 (targeting CCR6-dependent migration), and miR-98 (targeting IL-10) to enhance Treg proliferation, migration, and IL-10 production in vitro and in vivo. These salutary effects, in turn, lead to cardioprotection in MI (FIG. 8).

Genetic therapy, especially with RNA-based drugs, is becoming a promising strategy for the treatment of human diseases that are refractory to conventional approaches (Kim, 2020). Most RNA drugs are targeted therapies—such as small interfering (si) RNA, antisense RNA, and aptamers (Egli & Manoharan, 2023)—but these are only the tip of the iceberg for RNA-based approaches. Here a complementary approach was taken: mining EVs from therapeutically-active cells to identify ncRNA lead compounds, which can be used as they occur in nature or as bioinspiration for new chemical entities. EVsEVs

Recently, lncRNAs have emerged as potential modulators of gene expression in diverse cell lineages, with broad-ranging bioactivity (Boon et al, 2016). LncRNAs are >200 nt with limited or no coding potential. Various lncRNA isoforms act by binding and sequestering selected miRNAs (“miR sponge”), thereby releasing functional targets from miR translational suppression. These features of lncRNA are particularly pertinent to immune cells that exhibit dynamic functional plasticity in response to local microenvironments (Ahmad et al, 2020). Here, one particular lncRNA species-BCYRN1 was found to be highly enriched, being the most plentiful lncRNA in CDC-EVs. EVs

It was found that CDC-EV BCYRN1 could induce proliferation of human iTreg cells via sponging miR-138, enhancing ATG-7-dependent autophagy. Autophagy, a cellular process of self-degradation, regulates various components of the immune system, including natural killer cells, macrophages, dendritic cells, and T and B lymphocytes (Jiang et al, 2019). Without being bound by theory, this process can impact the homeostasis, survival, activation, proliferation, and differentiation of these immune cells, which are involved in both innate and adaptive immune responses (Jiang et al., 2019). ATG7 is a gene that helps form autophagosomes; interestingly, Treg cells lacking Atg7 exhibit increased apoptosis and rapidly lose expression of Foxp3, particularly following activation (Wei et al, 2016). In this study, it was found that miR-138 could target ATG7, resulting in inhibition of autophagy, which reduced iTreg proliferation. Overexpression of BCYRN1 attenuated miR-138-mediated downregulation of ATG7, promoting proliferation.

Regulatory T cells, like all other lymphocytes, migrate to sites of inflammation to carry out their regulatory function. Without being bound by theory, the expression of specific chemokine receptors on the surface of Tregs can facilitate their homing to these locations. Chemokine CCL20 has been linked to ischemic heart disease, specifically acute MI (Schumacher et al, 2021), insofar as levels of CCL20 in the blood of MI patients are higher than in healthy individuals (Safa et al, 2016). The receptor for CCL20, CCR6, plays a crucial role in the recruitment of Treg cells to the ischemic heart (Yamazaki et al., 2008). It was found that CDC-EVs induced upregulation of CCR6 and Treg migration, effects which were enhanced by overexpression of BCYRN1. RNA pull-down and luciferase assays revealed that BCYRN1 acts as a sponge for miR-150. While miR-150 suppressed CCR6 protein and migration of human iTregs, overexpression of BCYRN1 reversed this suppression. In short, it was demonstrated that CDC-EVs induce CCR6-dependent migration of Tregs via BCYRN1, which acts as a sponge for miR-150. The increase in cell migration may facilitate enhanced infiltration of injured cardiac tissue by Tregs.

IL-10, a pleiotropic cytokine that plays a crucial role in the regulation of immune responses, is produced by Tregs, monocytes, Th2 cells, subsets of activated T cells, and B cells (Wang et al, 2016). Treg-derived IL-10 is implicated in the preservation of immunotolerance and the maintenance of FOXP3 expression, stability, and associated regulatory mediators (Murai et al, 2009). EVsUsing gene knock-down and overexpression approaches, it was implicated that BCYRN1 was the key player in CDC-EV-mediated induction of IL-10 in human iTregs. Without being bound by theory, IL-10 upregulation can be explained by BCYRN1's ability to act as a sponge for miR-98, rationalizing how CDC-EVs induce IL-10 in human iTreg cells.

Modulating Tregs is a promising therapy for inflammatory diseases, utilizing the body's natural immune suppression mechanisms. More than 50 clinical trials (Roemhild et al, 2020) are currently exploring Treg autotransfusion for conditions including solid organ transplantation (to increase graft survival), graft-versus-host disease, and autoimmune disorders. However, many of these trials use a difficult and complex protocol that involves isolating and expanding Treg cells ex vivo before reinfusing them back into the patient. Not only does this introduce uncertainty regarding Tregs' suppression and growth capabilities after expansion, but also involves delays which undermine applicability to acute disease, such as MI or stroke. This in vivo data demonstrated that CDC-EVs and CDC-EV with BCYRN1 overexpression could induce infiltration, expanding IL-10+ Treg infiltration into the heart, which in turn, reduced infarct size and decreased concentration of cardiac troponin I (cTnI). The approach of using CDC-EVs, or CDC-EVs with BCYRN1 overexpression, to harness Treg cells in vivo, bypasses the limitations of ex vivo expansion.

### Example 8

This nonlimiting example shows BCYRN1 maintains suppressive capacity of hTreg in vitro.

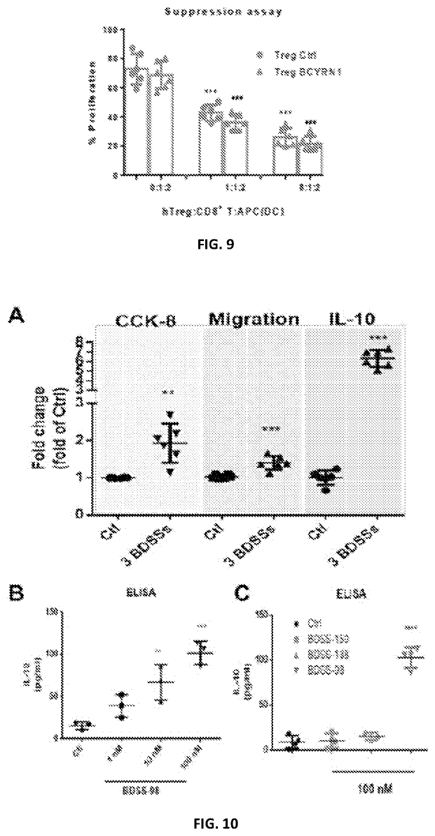

To determine if BCYRN1 impacts the ability of Tregs to suppress T-cell activation and antigen presenting cell activation (dendritic cells, DC), an in vitro assay was used with responder CD8+ T cells, DCs, and hTregs. Both unmodified hTreg (Treg Ctrl) and Treg BCYRN1 (hTreg transfected with BCYRN1) suppressed DC-mediated proliferation of CD8+ T cells (FIG. 9), verifying that BCYRN1 does not impede the central immunosuppressive phenotype of hTregs.

### Example 9

This nonlimiting example shows effects of BDSSs on Treg proliferation, migration, and IL10 production.

3 BDSS were prepared and admixed into lipid nanoparticles (LNPs, formed with transfection reagent DharmaFect; PerkinElmer). The BDSS included the following RNA: BDSS-138 (UCCCUCAAAGCAACAACCCCC (SEQ ID NO: 11)); BDSS-150 (GAGGCUAAGAGGCGGGAGGAU (SEQ ID NO: 12)); BDSS-98 (ACUUCCCUCAAAGCAACAACC (SEQ ID NO: 13)). LNPs containing 3 BDSSs induced Treg cell proliferation, migration, and IL-10 induction (FIG. 10 panel A).

Among the three BDSS species, BDSS-98 dose-dependently (FIG. 10 panel B) and uniquely upregulated IL-10 production (FIG. 10 panel C).

### Example 10

This nonlimiting example shows therapeutic efficacy of a BDSS cocktail in MI.

Equimolar amounts of all 3 BDSSs (as described in Example 9) were admixed into LNPs, presuming that the diverse BDSS species may act synergistically. MI was induced in 8-10-week-old C57BL/6 mouse by left coronary artery ligation for 45 minutes followed by reperfusion. Fifteen minutes later, the mice received (via tail vein) a BDSS cocktail (BDSS-138, BDSS-150 and BDSS-98, 0.5 mg/kg) or Mut-BDSS cocktail (binding site mutation). In vitro preliminary data indicated that an increase in autophagy underlies enhanced Treg proliferation, with BCYRN1 acting as a “sponge” to sequester miR-138.

Rapamycin is a potent inducer of autophagy in a diverse range of cell lines60; indeed, rapamycin, when added to the reperfusate, attenuates MI injury. To further test the role of autophagy, BDSS cocktail and rapamycin (positive control) were compared in terms of their ability to limit MI size.

Both BDSS cocktail and rapamycin reduced infarct mass and serum levels of cardiac troponin I (cTnl; FIG. 11). Thus, it was demonstrated that the disease-modifying bioactivity of BDSS species, in vitro and in vivo in a mouse MI model. The effects of BDSS are mimicked by, and comparable to, those of rapamycin, a known inducer of autophagy.

### Example 11

This nonlimiting example shows Treg depletion by CD25 antibody blocks the cardioprotective benefits of BDSS cocktail.

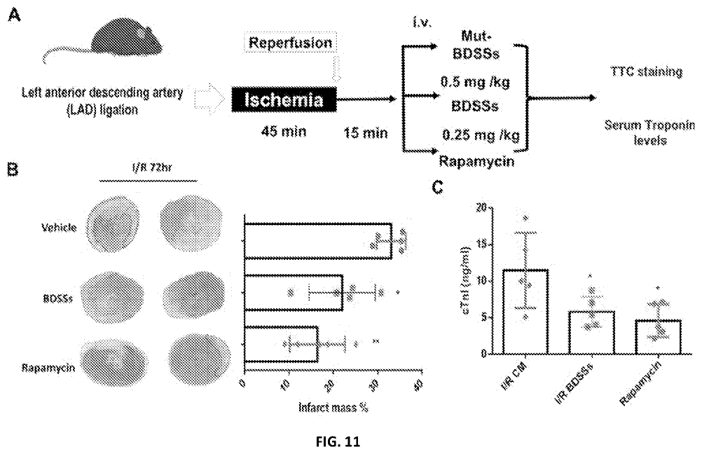

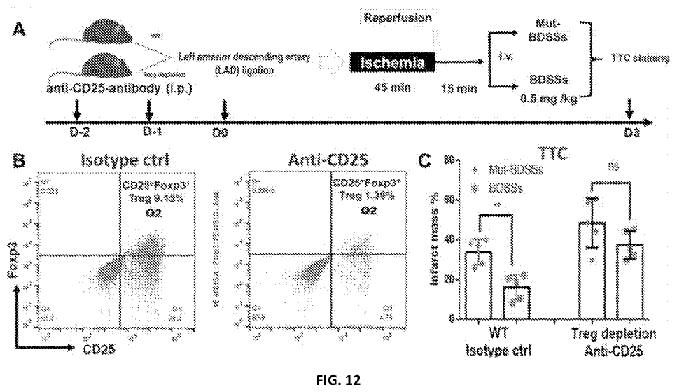

The benefits of BDSS cocktail post-MI were blocked by depletion of Tregs (FIG. 12). In a blinded study design, Tregs were depleted by daily intraperitoneal (i.p.) injection of anti-CD25 antibody (100 μg/mouse62) to achieve ˜80% Treg cell depletion (FIG. 12 panel B). WT (i.e., non-depleted) mice were given isotype control (rat IgG1). Afterwards, BDSS cocktail or mut-BDSS (with binding site mutations designed to eliminate miR sponging effects) were administered i.v. 15 min after reperfusion (as in FIG. 12 panel A). Hearts were excised 72 hours post-MI followed by TTC staining to quantify infarct mass histologically. Pooled data (FIG. 12 panel C) show that BDSS cocktail decreases infarct mass at 72 hours, and this effect is abrogated when Tregs are depleted. Thus, Tregs are required for BDSS's disease-modifying bioactivity.

### Example 13

This nonlimiting example shows CDC-EVs with BCYRN1 overexpressed.

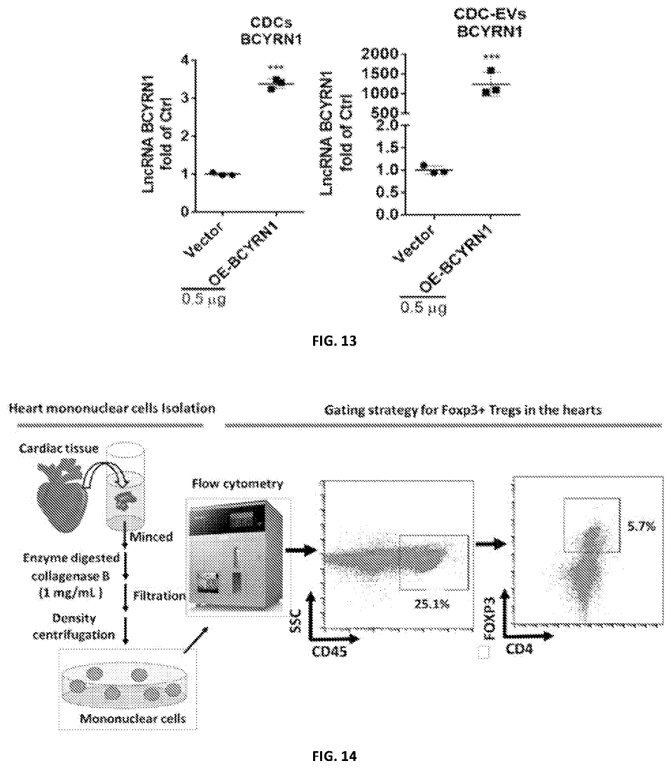

CDCs transduced with a BCYRN1 vector exhibited increased expression of BCYRN1 both in CDCs and in CDC-EVs (FIG. 13). The vector included a full length BCYRN1 cDNA in a lentivial vector (see Example 14).

### Example 14

This nonlimiting example shows experiments to explore the molecular mechanism(s) underlying BCYRN1-mediated human Treg cell proliferation, migration, and IL-10 production.

### Outline of Experimental Design:

1. Cells: Two human primary cell types can be used: CDCs and hTregs. Human CDCs are re-derived as needed from extensive existing frozen stocks. Each line has passed strict quality control, safety checks, and potency assay (improved recovery from MI in rats in vivo), as described. Human Treg cells, purchased from IQ Biosciences (Cat #IQB-Hu1-iTr-1), are obtained from normal volunteers. The purity and identity are confirmed by flow cytometry (90% CD4+CD25+(90% CD127, 85%% FOXP3+)), and the cells have negative assays for HIV, HCV, and Hepatitis B.

2. Vectors: BCYRN1 expression is driven by a CMV promoter, co-expressed with a GFP reporter, in a lentiviral vector that can be used for non-viral plasmid transfection of BCYRN1 in target cells (e.g., FIG. 1). Alternatively, the vector can be packaged into lentiviral particles for high efficiency transduction and stably integrated expression. Transient transfection is relied upon primarily, as hTregs are primary cells not requiring long-term genetic modification. The pLenti-CMV-GFP-2A-Puro-Blank Vector & BCYRN1 Lentivirus vector (Applied Biological Materials Inc.) are used.

3. Time points for cell assays: 0, 3, 6, 24, 48, 72 hours, unless otherwise indicated.

Experiment 14A: Investigate the Mechanism(s) by which BCYRN1 Mediates Proliferation of hTreg Cells.

Experiments are carried out to determine whether BCYRN1 acts as a miR sponge to sequester miR-138, promoting ATG7-dependent autophagy, which, in turn, enhances the proliferation of hTregs.

The following considerations motivate the present experiment: 1) CDC-EVs and BCYRN1 both induce proliferation of hTreg cells (FIG. 1); 2) BCYRN1 contains putative binding sites for sponging miR-138 (FIG. 4, panel E); 3) hTreg cells transfected with miR-138 show down-regulation of ATG-7 (FIG. 4, panel F); and 4) ATG-7 dependent autophagy is essential to the survival and proliferation of Treg cells. BCYRN1 may act as a miR-138 sponge to promote autophagy, which, in turn, favors hTreg proliferation.

The following is assayed: a) autophagy marker expression (LC3, P62, ATG7) using Western blotting (WB) and immunocytochemistry; b) proliferation assays (CCK8); c) qRT-PCR for quantification of BCYRN1 and miR-138; d) RNA pulldown and luciferase reporter assays are used to verify functional binding sequence of BCYRN1 targeting miR-138.

Experiment 14A1: Investigate the effect of BCYRN1 on autophagy in hTreg cells. Because autophagy plays a central role in promoting Treg cell proliferation, whether BCYRN1 induces autophagy in Treg cells is tested. Human Tregs are transfected with either empty vector or BCYRN1 overexpression vector for 48 hours followed by the assessment of autophagy markers using WB and immunocytochemistry.

Experiment 14A2: Test whether BCYRN1 functions as a sponge for miR-138. BCYRN1 contains a potential miR-138 binding site, and miR-138 targets ATG-7 (FIG. 4 panel E, F). To further test whether BCYRN1 functions as a sponge for miR-138, two experiments are conducted: 1. RNA pull-down assay. 5′ biotinylated BCYRN1 probe is added to hTreg cell lysate. After co-incubation, biotinylated BCYRN1 is precipitated using streptavidin followed by RNA extraction. The RNAs associated with BCYRN1 is assessed by qPCR for miR-138, and for BCYRN1 (positive control), GAPDH and U6 (both as negative controls); 2. Dual luciferase reporter assay. pmiR-GLO luciferase reporter plasmids containing the wildtype (WT) and a mutated (Mu) binding sequence of BCYRN1 for miR138 have been generated. hTreg cells is co-transfected with miR-138 mimic (in DharmaFECT LNPs) and a luciferase reporter construct (in DharmaFECT) for the specific target (WT or Mu), and luciferase activity 24 hours post-transfection is measured. Findings from these experiments determine whether BCYRN1 sponges miR-138 and further confirm the functional binding sequence of BCYRN1 for miR-138 (this 21 nt BDSS is tested in Example 15).

Experiment 14A3: Investigate the effect of BCYRN1 on miR-138/ATG7 dependent autophagy. Whether BCYRN1 attenuates miR-138-mediated downregulation of ATG-7 to promote proliferation of hTreg cells is tested. Four groups are tested: 1) hTregs transfected with mimic-control; 2) hTregs transfected with mimic-miR138 3) hTregs cotransfected with BCYRN1 overexpressing vector and mimic-control, and 4) hTregs co-transfected with BCYRN1 overexpressing vector and mimic-miR-138. All groups are assessed for expression of ATG7 (WB) and proliferation of hTregs (CCK-8).

Experiment 14A4: Investigate the effect of CDC-EVs with BCYRN1 overexpressed on proliferation of Tregs. As an alternative to plasmid vector transfection, whether overexpressing BCYRN1 in CDC-EVs can boost CDC-EV-mediated proliferation of Tregs is tested. CDCs are transfected with either empty vector or BCYRN1 lentiviral vector for 48 hours followed by EV isolation. It has been verified that CDCs transduced with BCYRN1 vector exhibit increased expression of BCYRN1 both in CDCs and in CDC-EVs (see Example 13). hTregs are exposed to CDC-EV-BCYRN1 (or CDC-EV-vector) followed by assessment of proliferation (CCK-8 assay). Findings from these experiments determine whether overexpressing BCYRN1 in CDC-EVs could improve CDC-EV-mediated induction of hTreg cell proliferation.

Two alternative approaches include: 1) a lentivirus is created and used for transfection of BCYRN1 in vitro and in vivo (as an alternative to this alternative, an AAV overexpressing BCYRN1 is made and tested); 2) A BDSS targeting miRNA-138 (BDSS-138) is created. Since this is only 21 nt in size, it can be readily synthesized and packaged into LNPs.

Experiment 14B: Investigate the Mechanism(s) by which BCYRN1 Enhances Migration of hTreg Cells.

Experiments are carried out to show that BCYRN1 acts as a sponge of miR-150 to promote CCR6-dependent migration of hTreg cells.

Preliminary data show: 1) CDC-EVs and BCYRN1 both enhance migration of hTreg cells (FIGS. 1, 3); 2) BCYRN1 contains putative binding sites for sponging miR-150 (FIG. 5); and 3) miR-138 targets CCR6 (FIG. 5). Because CCR6 plays a major role in recruiting Treg cells into inflammatory tissue, BCYRN1 may act as a miR-150 sponge to promote migration of hTreg cells.

The following are assayed: a) CCR6 expression using WB and qPCR; b) transwell migration assays; c) qPCR for quantification of BCYRN1 and miR-150; d) RNA pulldown and luciferase reporter assays are used to verify functional binding sequence of BCYRN1 targeting of miR-150.

Experiment 14B1: Test the concept that BCYRN1 functions as a sponge for miR-150. The following experiments are conducted: 1. RNA pull-down assay (as in Exp 14A2). Using qPCR, miR-150 is probed for in the RNA pulled down with BCYRN1; BCYRN1 (positive control) are also amplified, GAPDH and U6 (both as negative controls); 2. Dual luciferase reporter assay. pmiR-GLO luciferase reporter plasmids containing the wildtype (WT) & mutated (Mu) binding sequence of BCYRN1 for miR150 have been generated. Human Treg cells are co-transfected with miR-150 mimic (in DharmaFECT, as in FIG. 8) and the luciferase reporter construct (in DharmaFECT) for the specific target (WT/Mu) and luciferase activity is monitored for 24 hours post-transfection. These experiments determine whether BCYRN1 sponges miR-150 and further confirm the functional binding sequence of BCYRN1 for miR-150 (this BDSS-150 is tested in Example 15).

Experiment 14B2: Investigate the Effect of BCYRN1 on miR-150/CCR6 Dependent Migration of Treg Cells.

Four groups are tested: 1) hTreg cells transfected with mimic-control; group 2) hTreg cells transfected with mimic-miR150 3) hTreg cells co-transfected with BCYRN1 overexpressing vector and mimic-control, and 4) hTreg cells co-transfected with BCYRN1 overexpressing vector and mimic-miR-150. All re probed for expression of CCR6 (by WB) and migration of Treg cells (by transwell assay).

Experiment 14B3: Investigate the Effect of CDC-EVs with BCYRN1 Overexpressed on Treg Cell Migration.

Human Treg cells are exposed to either CDC-EV-vector or CDC-EV-BCYRN1 followed by assessment of Treg cell migration. This experiment determines whether BCYRN1 overexpression in CDC-EVs might boost the salutary effects of unfortified CDC-EVs on hTreg cells.

Experiment 14C: Investigate the Mechanism(s) by which BCYRN1 Increases IL-10 in hTreg Cells.

Experiments are carried out to determine whether BCYRN1 acts as a miR sponge of miR-98 to promote induction of IL-10 in hTreg cells.

The preliminary data show: 1) CDC-EVs and BCYRN1 both induce production of IL-10 in hTreg cells (FIGS. 1 and 3); 2) BCYRN1 contains a putative binding site for sponging miR-98 (FIG. 7A); 3) miR-98 targets IL10, as confirmed in transfected hTregs (FIG. 6).

The following are assayed: a) IL10 expression using qPCR and ELISA; b) qRT-PCR for quantification of BCYRN1 and miR-98; d) RNA pulldown and luciferase reporter assays are used to verify functional binding sequence of BCYRN1 targeting miR-98.

Experiment 14C1: Test the concept that BCYRN1 functions as a sponge for miR-98. The following experiments are conducted: 1. RNA pull-down assay (analogous to Experiment 14A2). Here, the following are amplified by qPCR, miR-98, BCYRN1 (positive control), GAPDH and U6 (both as negative controls); 2. Dual luciferase reporter assay. pmiR-GLO luciferase reporter plasmids containing the wildtype (WT) & mutated (Mu) binding sequence of the BCYRN1 for miR-98 have been generated. Human Treg cells are co-transfected with miR-98 mimic (DharmaFECT) and luciferase reporter construct (using transfection Reagent, DharmaFECT) for the specific target (WT/Mu) and luciferase activity is monitored for 24 h post-transfection. These experiments determine whether BCYRN1 sponges miR-150 and further confirm the functional binding sequence of BCYRN1 for miR-98 (this BDSS-98 will be tested in Experiment 15).

Experiment 14C2: Investigate the Effect of BCYRN1 on miR-98 Dependent IL-10 Production of Treg Cells.

Four groups are tested: 1) hTreg cells transfected with mimic-control; group 2) hTreg cells transfected with mimic-miR-98; 3) hTreg cells co-transfected with BCYRN1 overexpressing vector and mimic-control; and 4) hTreg cells co-transfected with BCYRN1 overexpressing vector and mimic-miR-98, for expression of IL-10 (by WB and ELISA).

Experiment 14C3: Investigate the effect of CDC-EVs with BCYRN1 overexpressed on the induction of IL-10 in Treg cells. Human Treg cells are exposed to either CDC-EV-vector or CDC-EV-BCYRN1 followed by the assessment of IL-10 induction in hTreg cells.

### Example 15

This nonlimiting example shows synthesizing the functional binding sequences of BCYRN1 (BCYRN1-Derived Short Sequence, BDSS) and testing their effects on Treg cell proliferation, migration, and IL-10 production in vitro.

All FDA-approved ncRNA drugs (n=12) are short (<35 nt) synthetic entities. Abbreviated forms of BCYRN1 which retain in vitro bioactivity are created and tested. The preliminary data (FIG. 10) showed that LNPs containing BDSS cocktail induce Treg cell proliferation, migration, and IL-10 induction. Among the three BDSS species, BDSS-98 uniquely upregulated IL-10 production. The other BDSS species individually are tested for their mechanism(s) and effects on hTreg proliferation, migration, and IL-10 production.

Synthetic RNA and transfection methods: BDSS-138, BDSS-150 and BDSS-98 are synthesized (Integrated DNA Technologies), as in previous experiments. Each BDSS is mixed with 5 μl of small RNA transfection reagent (DharmaFECT, as in FIG. 10) to a final volume of 100 μl in serum-free media. Following agitation and incubation (to form liposomal-RNA complexes), the preparation is added to Treg cell culture media to final concentrations of 1, 10, 100 nM.

Each BDSS is tested in the following experiments: hTreg cells transfected with BDSS-138, -150 or -98 are assessed for proliferation (Experiment 15-1, CCK-8 assay), autophagy pathway (Experiment 15-2, WB and immunostaining), migration (Experiment 15-3, transwell migration assay), CCR-6 dependent migration pathway (Experiment 15-4, WB, qPCR), and IL-10 production (Experiment 15-5, qPCR and ELISA). For these experiments synthesized mutated binding sequence (cf. FIGS. 11&12) is used as negative control. Dose-finding studies determine the minimal dosage that will suffice to achieve maximal bioactivity of each BDSS.

Experiments show that BDSS-138 affects proliferation/autophagy pathway, BDSS-150 CCR6-dependent migration, and BDSS-98 IL-10 production, with minimal or no effects of each on the other two processes. In some embodiments, admixtures of 2 or all 3 BDSSs are packaged into LNPs, as has been done before with cocktails of siRNAs, to capture the full therapeutic effect of BCYRN1. In some embodiments, chemical RNA modifications are introduced to increase stability. Among the alternative strategies, locked nucleic acid (LNA) modifications are tested. Non-Treg cell types may also be affected by BDSSs.

### Example 16

This nonlimiting example testing in vivo the therapeutic potential of BCYRN1 and BDSS in a CVB3-induced myocarditis mouse model.

The proposed in vivo experiments validate the in vitro findings and guide options for treating acute myocarditis.

Methods (End-point Measures): 1) Transthoracic echocardiography is performed to evaluate LV end systolic and end-diastolic dimensions, and LVEF; 2) hearts are stained with hematoxylin and eosin to evaluate the degree of inflammation; 3) heart infiltrating Tregs are assessed for IL-10 production and proliferation by flow cytometry. As shown in FIG. 14, hearts are minced into small tissue fragments followed by enzyme digestion with collagenase B (1 mg/mL, Roche) in HEPES buffer (rotated gently for 2 hours at 37° C.). Next, the cell suspensions are filtered with 40-μm cell strainers (BD Bioscience) mononuclear cells. Such cells are collected and stained with the following antibodies: anti-CD45, -CD4 and -Foxp3 (all from Biolegend). Viability of stained cells is determined using the Zombie Aqua Fixable Viability Kit (Biolegend) prior to fixation. Flow cytometry is performed on Sony SA3800 Cell Analyzer. First live single mononuclear cells are selected by gating on SSC and CD45+. Within this single cell population, cells are then further gated to select the double positive CD4+FoxP3+ Treg cell population. Using this strategy purified heart-infiltrating Treg cells are obtained for the analysis of IL-10 production (IL-10+FoxP3+) and proliferation (Brdu+FoxP3+).

### Experiment 16A: Assess In Vivo the Therapeutic Potential of BCYRN1 in a CVB3-Induced Myocarditis Mouse Model.

Experiment 16A1: Myocarditis is induced in six-to-eight-week-old A/J mice by CVB3 (50 TCID50/animal64) infection (saline recipients as controls). On day 14, the mice receive (i.v., via tail vein) CDC-EV-BCYRN1, CDCEV-Vector, CDC-EVs or vehicle (IMDM) (N=8-10 each group). At study endpoint on day 21, several functional endpoints are assessed: 1) Transthoracic echocardiography is performed on day 0 (baseline) and day 21 (endpoint). LV end-systolic dimensions, end-diastolic dimensions, and ejection fraction is evaluated in M-mode using parasternal short-axis views (Vevo 3100, VisualSonics). An average of three measurements for each animal are used. After in vivo phenotyping, hearts are collected under anesthesia for euthanasia. 2) Cardiac cross sections are frozen, fixed, and stained with hematoxylin and eosin. A semiquantitative assessment of inflammation is performed by a cardiac pathologist blinded to group assignment. Degree of inflammation is scored from 0-4, with 0 indicating no inflammatory infiltrates, 1 indicating small foci of inflammatory cells, 2 indicated larger foci containing >100 inflammatory cells, 3 indicating >10% of cross section involvement, and 4 indicating >30% of cross section involvement; and 3) infiltrating Tregs (by flow cytometry) are assessed for IL-10 production and proliferation. Since live cells cannot be isolated from hematoxylin- and eosin-stained heart, a parallel set of animals are used for flow cytometry analysis.

The experiments show whether the number of CD4+FOXP3+IL10+ cells is higher in animals that receive CDC-EV-BCYRN1, relative to vehicle and CDC-EV-vector controls, due to augmented Treg cell infiltration into the heart and increased production of IL-10 by Treg cells. CDC-EV-BCYRN1 increases the number of CD4+FOXP3+Brdu+ cells in the heart, by inducing Treg cell proliferation. As a result of these multiple effects on Tregs, CDC-EV-BCYRN1 treatment may improve LV ejection fraction and decrease cardiac inflammation. The cardioprotective effects of CDC-EV-BCYRN1 and CDC-EVs are compared to determine whether EV-BCYRN1 has augmented disease-modifying bioactivity relative to CDC-EVs (as might be predicted from the overexpression studies in FIG. 13). The 200 nt BCYRN1 is too long to make synthetically using current technology. Thus, alternative and/or supplementary approaches will include: 1) knocking down BCYRN1 in CDC-EVs to verify loss of function. This is achieved by transfecting CDCs with either siRNA-control or siRNA against BCYRN1 for 48 hours followed by EV isolation. In preliminary experiments, it has been found that transfection of CDCs with siRNA-BCYRN1 successfully decreases the expression of BCYRN1 both in CDCs and CDC-EVs (FIG. 9). After achieving BCYRN1 knockdown in CDCEVs, the effect of BCYRN1-depleted CDC-EVs in vivo is studied. 2) BCYRN1 lentivirus is created and its effects are tested directly in vivo, by infusing it intravenously.

### Experiment 16B: Assess In Vivo the Therapeutic Potential of BDSS in a CVB3-Induced Myocarditis Mouse Model.

Experimental design: The default strategy uses LNPs containing synthetic BDSS (singly or in combination, as informed by Example 15) with transfection reagent (DharmaFECT).

Experiment 16B1: Myocarditis is induced in 6-8-week old A/J mice by CVB3 (50 TCID50*/animal) infection (saline recipients as controls). On day 14, the mice receive (via tail vein) BDSS-138, BDSS-150, BDSS-98, combination of all 3 BDSSs (BDSS-138+150+98) or mut-BDSS (individual or combine) (N=8-10, each group). At termination on day 21 the same functional endpoints (as per Example 16A) are assessed.

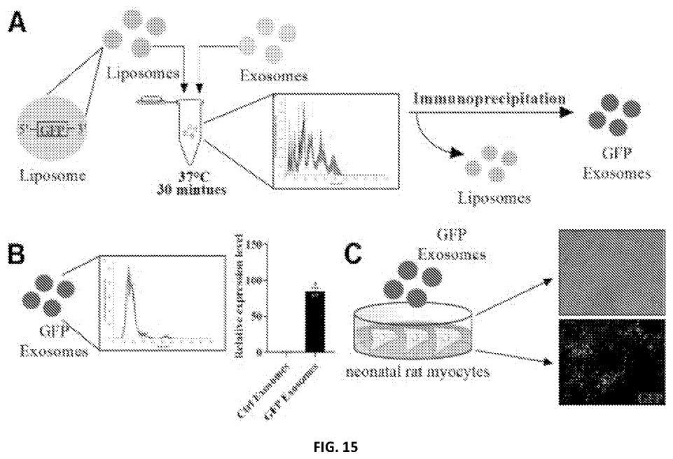

Experiments show that BDSS-150 augments Treg infiltration, while BDSS-138 and BDSS-98 increase proliferation and IL-10, respectively, in the heart. The cocktail of BDSS-138+150+98 enhances cardioprotection relative to each BDSS alone. Because these BDSS presumably act by sponging miRs, the mechanism resembles that of miRtargeted RNA interference. This technique can produce “off-target” effects caused by knockdown of genes other than the gene of interest. With this in mind, off target effects of BDSS TCs are looked for in further studies. An alternative approach include each BDSS, or all 3 BDSSs combined, loaded into CDC-EVs and immunoprecipitation used to select exosomes. This approach has been successfully implemented with several LNA-modified RNA payloads; FIG. 15 shows an example in which LNA-modified mRNA encoding GFP was loaded into CDC-EVs. Liposomes containing the BDSS are admixed with CDC-EVs at 37° C. in a shaker for 30 min. Liposome-exosome complex size distributions, measured by dynamic light scattering, reveal multiple peaks (FIG. 15 panel A, center), indicative of heterogeneous populations. To select exosomes, immunoprecipitation is used with anti-CD9, anti-CD63, and anti-CD81 antibodies (all of which target exosome-specific surface proteins90). As shown in FIG. 15 panel B, immunoprecipitants show a single peak (diameter ˜120 nm), characteristic of exosomes. By qPCR, GFP mRNA levels were markedly greater than in control exosomes. Addition of immunoprecipitated exosomes to neonatal rat cardiomyocytes in culture induced GFP expression, as observed by epifluorescence 24 hours later (FIG. 15 panel C), verifying the ability of functional transgene expression from modified GFP mRNA loaded into the exosomes. As an alternative to the strategy, these methods are used to load the BDSS (either singly or in combinations) into EVs.

### REFERENCES

Ahmad I, Valverde A, Ahmad F, Naqvi A R (2020) Long Noncoding RNA in Myeloid and Lymphoid Cell Differentiation, Polarization and Function. Cells 9
Akhmerov A, Rogers R, de Couto G, Valle J, Li L, Ibrahim A, Sanchez L, Zhang R, Lin Y N, Liu W et al (2021) Regulatory T cell activation, proliferation, and reprogramming induced by extracellular vesicles. J Heart Lung Transplant 40: 1387-1395
Boon R A, Jae N, Holdt L, Dimmeler S (2016) Long Noncoding RNAs: From Clinical Genetics to Therapeutic Targets?J Am Coll Cardiol 67: 1214-1226
Booy E P, McRae E K, Koul A, Lin F, McKenna S A (2017) The long non-coding RNA BC200 (BCYRN1) is critical for cancer cell survival and proliferation. Mol Cancer 16: 109
Bossi L, Figueroa-Bossi N (2016) Competing endogenous RNAs: a target-centric view of small RNA regulation in bacteria. Nat Rev Microbiol 14: 775-784
Briggs J A, Wolvetang E J, Mattick J S, Rinn J L, Barry G (2015) Mechanisms of Long Non-coding RNAs in Mammalian Nervous System Development, Plasticity, Disease, and Evolution. Neuron 88: 861-877
Cambier L, de Couto G, Ibrahim A, Echavez A K, Valle J, Liu W, Kreke M, Smith R R, Marban L, Marban E (2017) Y RNA fragment in extracellular vesicles confers cardioprotection via modulation of IL-10 expression and secretion. EMBO Mol Med 9: 337-352
Cambier L, Giani J F, Liu W, Ijichi T, Echavez A K, Valle J, Marban E (2018) Angiotensin II-Induced End-Organ Damage in Mice Is Attenuated by Human Exosomes and by an Exosomal Y RNA Fragment. Hypertension 72: 370-380
Chimenti I, Smith R R, Li T S, Gerstenblith G, Messina E, Giacomello A, Marban E (2010) Relative roles of direct regeneration versus paracrine effects of human cardiosphere-derived cells transplanted into infarcted mice. Circ Res 106: 971-980
Cook K W, Letley D P, Ingram R J, Staples E, Skjoldmose H, Atherton J C, Robinson K (2014) CCL20/CCR6-mediated migration of regulatory T cells to the Helicobacter pylori-infected human gastric mucosa. Gut 63: 1550-1559
Egli M, Manoharan M (2023) Chemistry, structure and function of approved oligonucleotide therapeutics. Nucleic Acids Res
Emanueli C, Shearn A I, Angelini G D, Sahoo S (2015) Exosomes and exosomal miRNAs in cardiovascular protection and repair. Vascul Pharmacol 71: 24-30
Hu G, Liao K, Yang L, Pendyala G, Kook Y, Fox H S, Buch S (2017) Tat-Mediated Induction of miRs-34a & -138 Promotes Astrocytic Activation via Downregulation of SIRTI: Implications for Aging in HAND. J Neuroimmune Pharmacol 12: 420-432
Huang F, Na N, Ijichi T, Wu X, Miyamoto K, Ciullo A, Tran M, Li L, Ibrahim A, Marban E et al (2021) Exosomally derived Y RNA fragment alleviates hypertrophic cardiomyopathy in transgenic mice. Mol Ther Nucleic Acids 24: 951-960
Ibrahim A, Marban E (2016) Exosomes: Fundamental Biology and Roles in Cardiovascular Physiology. Annu Rev Physiol 78: 67-83
Ibrahim A G, Cheng K, Marban E (2014) Exosomes as critical agents of cardiac regeneration triggered by cell therapy. Stem Cell Reports 2: 606-619
Ito M, Teshima K, Ikeda S, Kitadate A, Watanabe A, Nara M, Yamashita J, Ohshima K, Sawada K, Tagawa H (2014) MicroRNA-150 inhibits tumor invasion and metastasis by targeting the chemokine receptor CCR6, in advanced cutaneous T-cell lymphoma. Blood 123: 1499-1511
Jeggari A, Marks D S, Larsson E (2012) miRcode: a map of putative microRNA target sites in the long non-coding transcriptome. Bioinformatics 28: 2062-2063
Jiang G M, Tan Y, Wang H, Peng L, Chen H T, Meng X J, Li L L, Liu Y, Li W F, Shan H (2019) The relationship between autophagy and the immune system and its applications for tumor immunotherapy. Mol Cancer 18: 17
Khan M, Nickoloff E, Abramova T, Johnson J, Verma S K, Krishnamurthy P, Mackie A R, Vaughan E, Garikipati V N, Benedict C et al (2015) Embryonic stem cell-derived exosomes promote endogenous repair mechanisms and enhance cardiac function following myocardial infarction. Circ Res 117: 52-64
Kim Y K (2020) RNA Therapy: Current Status and Future Potential. Chonnam Med J 56: 87-93
Kishore R, Khan M (2016) More Than Tiny Sacks: Stem Cell Exosomes as Cell-Free Modality for Cardiac Repair. Circ Res 118: 330-343
Lang N, Wang C, Zhao J, Shi F, Wu T, Cao H (2020) Long non-coding RNA BCYRN1 promotes glycolysis and tumor progression by regulating the miR-149/PKM2 axis in non-small-cell lung cancer. Mol Med Rep 21: 1509-1516
Li J, Tan J, Martino M M, Lui K O (2018) Regulatory T-Cells: Potential Regulator of Tissue Repair and Regeneration. Front Immunol 9: 585
Liao K, Niu F, Dagur R S, He M, Tian C, Hu G (2020) Intranasal Delivery of lincRNA-Cox2 siRNA Loaded Extracellular Vesicles Decreases Lipopolysaccharide-Induced Microglial Proliferation in Mice. J Neuroimmune Pharmacol 15: 390-399
Lim H W, Broxmeyer H E, Kim C H (2006) Regulation of trafficking receptor expression in human forkhead box P3+ regulatory T cells. J Immunol 177: 840-851
Liu Y, Chen Q, Song Y, Lai L, Wang J, Yu H, Cao X, Wang Q (2011) MicroRNA-98 negatively regulates IL-10 production and endotoxin tolerance in macrophages after LPS stimulation. FEBS Lett 585: 1963-1968
MacDonald K N, Piret J M, Levings M K (2019) Methods to manufacture regulatory T cells for cell therapy. Clin Exp Immunol 197: 52-63
Makkar R R, Smith R R, Cheng K, Malliaras K, Thomson L E, Berman D, Czer L S, Marban L, Mendizabal A, Johnston P V et al (2012) Intracoronary cardiosphere-derived cells for heart regeneration after myocardial infarction (CADUCEUS): a prospective, randomised phase 1 trial. Lancet 379: 895-904
Malliaras K, Li T S, Luthringer D, Terrovitis J, Cheng K, Chakravarty T, Galang G, Zhang Y, Schoenhoff F, Van Eyk J et al (2012) Safety and efficacy of allogeneic cell therapy in infarcted rats transplanted with mismatched cardiosphere-derived cells. Circulation 125: 100-112
Marban E (2018) A mechanistic roadmap for the clinical application of cardiac cell therapies. Nat Biomed Eng 2: 353-361
McDonald C M, Marban E, Hendrix S, Hogan N, Ruckdeschel Smith R, Eagle M, Finkel R S, Tian C, Janas J, Harmelink M M et al (2022) Repeated intravenous cardiosphere-derived cell therapy in late-stage Duchenne muscular dystrophy (HOPE-2): a multicentre, randomised, double-blind, placebo-controlled, phase 2 trial. Lancet 399: 1049-1058
Mu M, Niu W, Zhang X, Hu S, Niu C (2020) LncRNA BCYRN1 inhibits glioma tumorigenesis by competitively binding with miR-619-5p to regulate CUEDC2 expression and the PTEN/AKT/p21 pathway. Oncogene 39: 6879-6892
Murai M, Turovskaya O, Kim G, Madan R, Karp C L, Cheroutre H, Kronenberg M (2009) Interleukin 10 acts on regulatory T cells to maintain expression of the transcription factor Foxp3 and suppressive function in mice with colitis. Nat Immunol 10: 1178-1184
Peet C, Ivetic A, Bromage D I, Shah A M (2020) Cardiac monocytes and macrophages after myocardial infarction. Cardiovasc Res 116: 1101-1112
Puhl S L, Steffens S (2019) Neutrophils in Post-myocardial Infarction Inflammation: Damage vs. Resolution?Front Cardiovasc Med 6: 25
Raffin C, Vo L T, Bluestone J A (2020) T(reg) cell-based therapies: challenges and perspectives. Nat Rev Immunol 20: 158-172
Roemhild A, Otto N M, Moll G, Abou-El-Enein M, Kaiser D, Bold G, Schachtner T, Choi M, Oellinger R, Landwehr-Kenzel S et al (2020) Regulatory T cells for minimising immune suppression in kidney transplantation: phase I/IIa clinical trial. BMJ 371: m3734
Romano M, Fanelli G, Albany C J, Giganti G, Lombardi G (2019) Past, Present, and Future of Regulatory T Cell Therapy in Transplantation and Autoimmunity. Front Immunol 10: 43
Safa A, Rashidinejad H R, Khalili M, Dabiri S, Nemati M, Mohammadi M M, Jafarzadeh A (2016) Higher circulating levels of chemokines CXCL10, CCL20 and CCL22 in patients with ischemic heart disease. Cytokine 83: 147-157
Sahoo S, Klychko E, Thorne T, Misener S, Schultz K M, Millay M, Ito A, Liu T, Kamide C, Agrawal H et al (2011) Exosomes from human CD34(+) stem cells mediate their proangiogenic paracrine activity. Circ Res 109: 724-728
Sahoo S, Losordo D W (2014) Exosomes and cardiac repair after myocardial infarction. Circ Res 114: 333-344
Schumacher D, Liehn E A, Singh A, Curaj A, Wijnands E, Lira S A, Tacke F, Jankowski J, Biessen E A L, van der Vorst E P C (2021) CCR6 Deficiency Increases Infarct Size after Murine Acute Myocardial Infarction. Biomedicines 9
Skryabin B V, Sukonina V, Jordan U, Lewejohann L, Sachser N, Muslimov I, Tiedge H, Brosius J (2003) Neuronal untranslated BC1 RNA: targeted gene elimination in mice. Mol Cell Biol 23: 6435-6441
Sun K, Li Y Y, Jin J (2021) A double-edged sword of immuno-microenvironment in cardiac homeostasis and injury repair. Signal Transduct Target Ther 6: 79
Tang T T, Yuan J, Zhu Z F, Zhang W C, Xiao H, Xia N, Yan X X, Nie S F, Liu J, Zhou S F et al (2012) Regulatory T cells ameliorate cardiac remodeling after myocardial infarction. Basic Res Cardiol 107: 232
Tsao C W, Aday A W, Almarzooq Z I, Alonso A, Beaton A Z, Bittencourt M S, Boehme A K, Buxton A E, Carson A P, Commodore-Mensah Y et al (2022) Heart Disease and Stroke Statistics-2022 Update: A Report From the American Heart Association. Circulation 145: e153-e639
Wang H, Tacoangeli A, Popp S, Muslimov I A, Imataka H, Sonenberg N, Lomakin T B, Tiedge H (2002) Dendritic B C1 RNA: functional role in regulation of translation initiation. J Neurosci 22: 10232-10241
Wang L, Das J K, Kumar A, Peng H Y, Ren Y, Xiong X, Yang J M, Song J (2021) Autophagy in T-cell differentiation, survival and memory. Immunol Cell Biol 99: 351-360
Wang S, Gao X, Shen G, Wang W, Li J, Zhao J, Wei Y Q, Edwards C K (2016) Interleukin-10 deficiency impairs regulatory T cell-derived neuropilin-1 functions and promotes Th1 and Th17 immunity. Sci Rep 6: 24249
Wei J, Long L, Yang K, Guy C, Shrestha S, Chen Z, Wu C, Vogel P, Neale G, Green D R et al (2016) Autophagy enforces functional integrity of regulatory T cells by coupling environmental cues and metabolic homeostasis. Nat Immunol 17: 277-285
Weirather J, Hofmann U D, Beyersdorf N, Ramos G C, Vogel B, Frey A, Ertl G, Kerkau T, Frantz S (2014) Foxp3+CD4+ T cells improve healing after myocardial infarction by modulating monocyte/macrophage differentiation. Circ Res 115: 55-67
Yamazaki T, Yang X O, Chung Y, Fukunaga A, Nurieva R, Pappu B, Martin-Orozco N, Kang H S, Ma L, Panopoulos A D et al (2008) CCR6 regulates the migration of inflammatory and regulatory T cells. J Immunol 181: 8391-8401
Zhong J, Chuang S C, Bianchi R, Zhao W, Lee H, Fenton A A, Wong R K, Tiedge H (2009) BC1 regulation of metabotropic glutamate receptor-mediated neuronal excitability. J Neurosci 29: 9977-9986

Although the foregoing has been described in some detail by way of illustrations and examples for purposes of clarity and understanding, it will be understood by those of skill in the art that modifications can be made without departing from the spirit of the present disclosure. Therefore, it should be understood that the forms disclosed herein are illustrative only and are not intended to limit the scope of the present disclosure, but rather to also cover all modification and alternatives coming with the true scope and spirit of the embodiments of the present disclosure.

It is contemplated that various combinations or subcombinations of the specific features and aspects of the embodiments disclosed above may be made. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed subject matter. Thus, it is intended that the scope of the present disclosure should not be limited by the particular disclosed embodiments described above. Moreover, while the disclosed subject matter is susceptible to various modifications, and alternative forms, specific examples thereof have been shown in the drawings and are herein described in detail. It should be understood, however, that the present disclosure is not to be limited to the particular forms or methods disclosed, but is to cover all modifications, equivalents, and alternatives falling within the spirit and scope of the various embodiments described and the appended claims.

Any methods disclosed herein need not be performed in the order recited. The methods disclosed herein include certain actions taken by a practitioner; however, they can also include any third-party instruction of those actions, either expressly or by implication. For example, actions such as “administering to a subject in need of treating a heart condition or symptom thereof a therapeutically effective amount of the nucleic acid” include “instructing the administration of an effective amount of the nucleic acid to a subject.” In addition, where features or aspects of the disclosure are described in terms of Markush groups, those skilled in the art will recognize that the disclosure is also thereby described in terms of any individual member or subgroup of members of the Markush group.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 90%” includes “90%.” In some embodiments, at least 95% homologous includes 96%, 97%, 98%, 99%, and 100% homologous to the reference sequence. In addition, when a sequence is disclosed as “comprising” a nucleotide or amino acid sequence, such a reference shall also include, unless otherwise indicated, that the sequence “comprises”, “consists of” or “consists essentially of” the recited sequence.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like.

All patents and other publications; including literature references, issued patents, published patent applications, and co-pending patent applications cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicant and does not constitute any admission as to the correctness of the dates or contents of these documents.

1. A therapeutic composition comprising at least one nucleic acid comprising a BDSS (BCYRN1-derived binding sequence), the BDSS comprising a nucleotide sequence as provided by any one of SEQ ID NOs: 11-16, or a sequence at least 91% identical thereto, wherein each of the at least one nucleic acid is no more than 35 nucleotides in length.

2. (canceled)

3. The composition of claim 1, wherein the BDSS comprises a nucleotide sequence as provided by any one of SEQ ID NOs: 11-16.

4.-7. (canceled)

8. The composition of claim 1, comprising at least one of:
a first nucleic acid comprising a BDSS comprising: UCCCUCAAAGCAACAACCCCC (SEQ ID NO: 11), or a sequence at least 91% identical thereto, wherein the first nucleic acid is no more than 35 nucleotides in length;
a second nucleic acid comprising a BDSS comprising: GAGGCUAAGAGGCGGGAGGAU (SEQ ID NO: 12), or a sequence at least 91% identical thereto, wherein the second nucleic acid is no more than 35 nucleotides in length; and
a third nucleic acid comprising a BDSS comprising: ACUUCCCUCAAAGCAACAACC (SEQ ID NO: 13), or a sequence at least 91% identical thereto, wherein the third nucleic acid is no more than 35 nucleotides in length.

9. The composition of claim 8, comprising at least two of the first, second and third nucleic acids.

10.-21. (canceled)

22. The composition of claim 1, wherein the nucleotide sequence comprises ACAAC (SEQ ID NO: 18).

23. The composition of claim 1, wherein the nucleotide sequence comprises GGGAG (SEQ ID NO: 19).

24. The composition of claim 1, wherein the at least one nucleic acid or BDSS compound comprises RNA.

25. The composition of claim 1, wherein the at least one nucleic acid or BDSS compound comprises at least one chemically modified nucleotide.

26.-28. (canceled)

29. The therapeutic composition of claim 1, comprising:
a first BDSS (BCYRN1-derived binding sequence) compound as provided by SEQ ID NO:11, wherein the first BDSS compound is no more than 35 nucleotides in length;
a second BDSS compound as provided by SEQ ID NO:12, wherein the second BDSS compound is no more than 35 nucleotides in length;
a third BDSS compound as provided by SEQ ID NO:13, wherein the third BDSS compound is no more than 35 nucleotides in length; and
a pharmaceutically acceptable excipient.

30. The composition of claim 1, comprising a transfection reagent.

31. (canceled)

32. (canceled)

33. An isolated nucleic acid comprising a nucleotide sequence of:
(SEQ ID NO: 11)
UCCCUCAAAGCAACAACCCCC;
(SEQ ID NO: 12)
GAGGCUAAGAGGCGGGAGGAU;
or
(SEQ ID NO: 13)
ACUUCCCUCAAAGCAACAACC,
wherein the nucleic acid is RNA, wherein the nucleic acid is at most 35 nucleotides long.

34.-40. (canceled)

41. A vector comprising the nucleic acid of claim 33.

42. (canceled)

43. (canceled)

44. A regulatory T cell comprising the nucleic acid of claim 33, wherein an anti-inflammatory activity of the regulatory T cell is increased compared to a regulatory T cell without the nucleic acid.

45. A regulatory T cell genetically modified to comprise the vector of claim 41.

46. The regulatory T cell of claim 44, wherein the cell is in a subject.

47. The regulatory T cell of claim 44, wherein the cell is in culture.

48. (canceled)

49. A method of treating an immune-related disorder, comprising administering a therapeutically effective amount of the composition of claim 1 to a subject in need thereof, to thereby treat an immune-related disorder.

50.-55. (canceled)

56. The method of claim 49, wherein the immune-related disorder comprises a cardiac immune-related disorder, an autoimmune disease, or transplant rejection.

57. (canceled)

58. The method of claim 49, wherein the immune-related disorder comprises one or more of myocarditis, myocardial infarction, autoimmune conditions or an inflammatory conditions associated with a viral infection.

59. (canceled)

60. (canceled)

61. A method of modulating regulatory T-cell (Treg) activity, comprising contacting Tregs with an effective amount of a noncoding RNA BCYRN1 or a derivative thereof, to thereby modulate the activity of the Tregs.

62.-72. (canceled)
